(19) United States
(12) Patent Application Publication
(10) Pub. No.: US 2012/0330012 A1
(43) Pub. Date: Dec. 27, 2012

(54) Novel Tricyclic Compounds

(75) Inventors: Kristine E. Frank, Grayslake, IL (US); Andrew Burchat, Shrewsbury, MA (US); Philip Cox, Grayslake, IL (US); David C. Ihle, Worcester, MA (US); Kelly D. Mullen, Charlton, MA (US); Gagandeep Somal, Framingham, MA (US); Anil Vasudevan, Union Grove, WI (US); Lu Wang, Westborough, MA (US); Noel S. Wilson, Kenosha, WI (US)

(73) Assignee: ABBOTT LABORATORIES, Abbott Park, IL (US)

(21) Appl. No.: 13/458,101

(22) Filed: Apr. 27, 2012

Related U.S. Application Data
(60) Provisional application No. 61/480,524, filed on Apr. 29, 2011.

(51) Int. Cl. C07D 487/14

(52) U.S. Cl. 544/251

(57) ABSTRACT

The invention provides compounds of Formula (I)

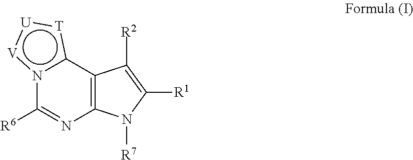

pharmaceutically acceptable salts, pro-drugs, biologically active metabolites, stereoisomers and isomers thereof wherein the variable are defined herein. The compounds of the invention are useful for treating immunological and oncological conditions.

### CROSS REFERENCE TO RELATED APPLICATION

This application claims priority to U.S. Provisional Application Ser. No. 61/480,524 filed on Apr. 29, 2011, the contents of which are incorporated herein.

### BACKGROUND OF THE INVENTION

The invention provides a novel class of compounds, pharmaceutical compositions comprising such compounds and methods of using such compounds to treat or prevent diseases or disorders associated with abnormal or deregulated kinase activity, particularly diseases or disorders that involve abnormal activation of the Jak1, Jak2, Jak3, Tyk2, KDR, Flt-3, CDK2, CDK4, TANK, Trk, FAK, Abl, Bcr-Abl, cMet, b-RAF, FGFR3, c-kit, PDGF-R, Syk, BTK, CSF1R, PKC kinases or Aurora kinases.

The protein kinases represent a large family of proteins that play a central role in the regulation of a wide variety of cellular processes and maintenance of cellular function. A partial, non-limiting, list of these kinases include: non-receptor tyrosine kinases such as the Janus kinase family (Jak1, Jak2, Jak3 and Tyk2); the fusion kinases, such as BCR-Abl, focal adhesion kinase (FAK), Fes, Lck and Syk; receptor tyrosine kinases such as platelet-derived growth factor receptor kinase (PDGF-R), the receptor kinase for stem cell factor, c-kit, the hepatocyte growth factor receptor, c-Met, and the fibroblast growth factor receptor, FGFR3; and serine/threonine kinases such as b-RAF, mitogen-activated protein kinases (e.g., MKK6) and SAPK2β. Aberrant kinase activity has been observed in many disease states including benign and malignant proliferative disorders as well as diseases resulting from inappropriate activation of the immune and nervous systems. The novel compounds of this invention inhibit the activity of one or more protein kinases and are, therefore, expected to be useful in the treatment of kinase-mediated diseases.

### SUMMARY OF THE INVENTION

In a first embodiment the invention provides a compound of Formula (I)

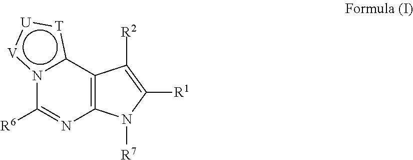

pharmaceutically acceptable salts, pro-drugs and biologically active metabolites thereof wherein

T is N or CR³;

U is N or CR⁴;

V is N or CR⁵;

R¹ is H, optionally substituted (C₁-C₆)alkyl, optionally substituted (C₃-C₆)cycloalkyl, optionally substituted heterocyclyl, optionally substituted aryl, or optionally substituted heteroaryl;

R² is H or optionally substituted (C₁-C₆)alkyl, NR^{a}R^{b}, OR^{b}, CONR^{a}R^{b}, NR^{a}COR^{b}, optionally substituted (C₃-C₆)cycloalkyl, optionally substituted heterocyclyl, optionally substituted aryl, or optionally substituted heteroaryl;

R³ is H, Br, Cl, F, optionally substituted (C₁-C₆)alkyl, optionally substituted (C₃-C₆)cycloalkyl, optionally substituted heterocyclyl, optionally substituted aryl, or optionally substituted heteroaryl;

R⁴ is H, Br, Cl, F, optionally substituted (C₁-C₆)alkyl, optionally substituted (C₃-C₆)cycloalkyl, optionally substituted heterocyclyl, optionally substituted aryl, or optionally substituted heteroaryl;

R⁵ is H, Br, Cl, F, optionally substituted (C₁-C₆)alkyl, optionally substituted (C₃-C₆)cycloalkyl, optionally substituted heterocyclyl, optionally substituted aryl, or optionally substituted heteroaryl;

R⁶ is H or NR^{a}R^{b};

R⁷ is H, optionally substituted-(CH₂)ₙ—P(═O)(OR^{a})(OR^{a}), optionally substituted —(CH₂)ₙ—O—P(═O)(OR^{a})(OR^{a}), optionally substituted-(CH₂)ₙ—P(═O)(OR^{a})(R^{a}), —CH═CH—P(═O)(OR^{a})(OR^{a});

R^{a} is H;

R^{b} is H, optionally substituted (C₁-C₆)alkyl, optionally substituted aryl, optionally substituted heteroaryl, optionally substituted (C₃-C₆)cycloalkyl, or optionally substituted heterocyclyl; and

n is 0, 1 or 2.

In a second embodiment the invention provides a compound according to the first embodiment wherein

R¹ is H or optionally substituted heteroaryl;

R³ is H, optionally substituted (C₁-C₆)alkyl, optionally substituted (C₃-C₆)cycloalkyl, or optionally substituted heterocyclyl;

R⁴ is H, optionally substituted (C₁-C₆)alkyl or optionally substituted pyridinyl; and

R⁵ is H, Br, or optionally substituted phenyl.

In a third embodiment the invention provides a compound according to any of the foregoing embodiments wherein T is N.

In a fourth embodiment the invention provides a compound according to any of the foregoing embodiments wherein U is CR⁴.

In a fifth embodiment the invention provides a compound according to any of the foregoing embodiments wherein V is CR⁵.

In a sixth embodiment the invention provides a compound according to any of the foregoing embodiments wherein R⁵ is Br or optionally substituted phenyl.

In a seventh embodiment the invention provides a compound according to any of the foregoing embodiments wherein R⁶ is NR^{a}R^{b} wherein R^{a} is H and R^{b} is optionally substituted phenyl.

In an eighth embodiment the invention provides a compound according to any of the foregoing embodiments wherein R¹ is optionally substituted pyridinyl.

In a ninth embodiment the invention provides a compound according to any of the foregoing embodiments wherein R⁴ is optionally substituted phenyl or optionally substituted pyridinyl.

In a tenth embodiment the invention provides a compound according to the third embodiment wherein U is N.

In an eleventh embodiment the invention provides a compound according to the eleventh embodiment wherein V is CR⁵.

In a twelfth embodiment the invention provides a compound according to the eleventh embodiment wherein R⁵ is optionally substituted phenyl.

In a thirteenth embodiment the invention provides a compound according to the second embodiment wherein T is CR³.

In a fourteenth embodiment the invention provides a compound of according to the thirteenth embodiment wherein U is N.

In a fifteenth embodiment the invention provides a compound according to the fourteenth embodiment wherein V is N.

In a sixteenth embodiment the invention provides a compound according to the fifteenth embodiment wherein R³ is optionally substituted cyclohexyl or optionally substituted piperidinyl.

In a seventeenth embodiment the invention provides a compound according to the fourteenth embodiment wherein V is CR⁵.

In an eighteenth embodiment the invention provides a compound of according to the seventeenth embodiment wherein R³ is optionally substituted cyclohexyl or optionally substituted piperidinyl and R⁵ is H, optionally substituted (C₁-C₆)alkyl or optionally substituted phenyl.

In a nineteenth embodiment the invention provides a compound according to the foregoing embodiments wherein the compound is

1-Cyclohexyl-7H-imidazo[1,5-c]pyrrolo[3,2-e]pyrimidine;

1-Cyclohexyl-7H-pyrrolo[3,2-e][1,2,3]triazolo[1,5-c]pyrimidine;

Benzyl 3 -(3 -isopropyl-7H-imidazo[1,5-c]pyrrolo[3,2-e]pyrimidin-1-yl)piperidine-1-carboxylate;

Benzyl 3-(7H-imidazo[1,5-c]pyrrolo[3,2-e]pyrimidin-1-yl)piperidine-1-carboxylate;

(S)-1-(3-(7H-Pyrrolo[3,2-e][1,2,3]triazolo [1,5-c]pyrimidin-1-yl)piperidine-1-carbonyl)cyclopropanecarbonitrile;

(R)-1-(3-(7H-pyrrolo[3,2-e][1,2,3]triazolo[1,5-c]pyrimidin-1-yl)piperidine-1-carbonyl)cyclopropanecarbonitrile;

4-(7H-Imidazo[1,2-c]pyrrolo[3,2-e]pyrimidin-5-ylamino)-N-propylbenzamide;

3-(4-Methoxyphenyl)-7H-pyrrolo[3,2-e][1,2,4]triazolo[4,3-c]pyrimidine;

3-(4-(Methylsulfonyl)phenyl)-7H-pyrrolo[3,2-e][1,2,4]triazolo[4,3-c]pyrimidine;

2-(4-(7H-Imidazo[1,2-c]pyrrolo[3,2-e]pyrimidin-3-yl)phenyl)propan-2-ol;

2-(Pyridin-3-yl)-7H-imidazo[1,2-c]pyrrolo[3,2-e]pyrimidine;

8-(Pyridin-4-yl)-7H-imidazo[1,2-c]pyrrolo[3,2-e]pyrimidine; or

1-(3-(3-(4-isopropylphenyl)-7H-imidazo[1,5-c]pyrrolo[3,2-e]pyrimidin-1-yl)piperidin-1-yl)ethanone.

### DETAILED DESCRIPTION OF THE INVENTION

Protein kinases are a broad and diverse class, of over 500 enzymes, that include oncogenes, growth factors receptors, signal transduction intermediates, apoptosis related kinases and cyclin dependent kinases. They are responsible for the transfer of a phosphate group to specific tyrosine, serine or threonine amino acid residues, and are broadly classified as tyrosine and serine/threonine kinases as a result of their substrate specificity.

The Jak family kinases (Jak1, Jak2, Jak3 and Tyk2) are cytoplasmic tyrosine kinases that associate with membrane bound cytokine receptors. Cytokine binding to their receptor initiates Jak kinase activation via trans and autophosphorylation processes. The activated Jak kinases phosphorylate residues on the cytokine receptors creating phosphotyrosine binding sites for SH2 domain containing proteins such as Signal Transduction Activators of Transcript (STAT) factors and other signal regulators transduction such as suppressor of cytokine signaling (SOCS) proteins and SH2 domain-containing inositol 5′-phosphatases (SHIP). Activation of STAT factors via this process leads to their dimerization, nuclear translocation and new mRNA transcription resulting in expression of immunocyte proliferation and survival factors as well as additional cytokines, chemokines and molecules that facilitate cellular trafficking (see Journal of Immunology, 2007, 178, p. 2623). Jak kinases transduce signals for many different cytokine families and hence potentially play roles in diseases with widely different pathologies including but not limited to the following examples. Both Jak1 and Jak3 control signaling of the so-called common gamma chain cytokines (IL2, IL4, IL7, IL9, IL15 and IL21), hence simultaneous inhibition of either Jak1 or Jak3 could be predicted to impact Th1 mediated diseases such as rheumatoid arthritis via blockade of IL2, IL7 and IL15 signaling. On the other hand, IL2 signaling has recently been shown to be essential for development and homeostasis of T-regulatory cells (Malek T R et al., Immunity, 2002, 17(2), p. 167-78). Thus, based on genetic data, blockade of IL2 signaling alone is predicted to result in autoimmunity (Yamanouchi J et al., Nat Genet., 2007, 39(3), p. 329-37, and Willerford D M et al., Immunity, 1995, 3(4), p. 521-30). Th2 mediated diseases such as asthma or atopic dermatitis via IL4 and IL9 signaling blockade. Jak1 and Tyk2 mediate signaling of IL13 (see Int. Immunity, 2000, 12, p. 1499). Hence, blockade of these may also be predicted to have a therapeutic effect in asthma. These two kinases are also thought to mediate Type I interferon signaling; their blockade could therefore be predicted to reduce the severity of systemic lupus erythematosus (SLE). Tyk2 and Jak2 mediate signaling of IL12 and IL23. In fact, blockade of these cytokines using monoclonal antibodies has been effective in treating psoriasis. Therefore blockade of this pathway using inhibitors of these kinases could be predicted to be effective in psoriasis as well. In summary, this invention describes small-molecule compounds that inhibit, regulate and/or modulate Jak family kinase activity that is pivotal to several mechanisms thought critical to the progression of autoimmune diseases including, but not limited to, rheumatoid arthritis (RA), systemic lupus erythematosus (SLE), multiple sclerosis (MS), Crohn's disease, psoriasis, psoriatic arthritis, juvenile idiopathic arthritis, plaque psoriasis, polyarticular juvenile idiopathic arthritis, ankylosing spondylitis and asthma.

Several pathologically significant cytokines signal via Jak1 alone (Guschin D, et al., EMBO J. 1995 Apr. 3; 14(7):1421-9; Parganas E, et al., Cell. 1998 May 1;93(3):385-95; Rodig S. J., et al., Cell. 1998 May 1; 93(3):373-83). Blockade of one of these, IL6, using an IL6R neutralizing antibody, has been shown to significantly improve disease scores in human rheumatoid arthritis patients (Nishimoto N. et al., Ann Rheum Dis., 2007, 66(9), p. 1162-7). Similarly, blockade of GCSF signaling, which is also mediated by Jak1 alone, using neutralizing monoclonal antibodies or target gene deletion protects mice from experimental arthritis (Lawlor K. E. et al., Proc Natl Acad Sci U.S.A., 2004, 101(31), p. 11398-403). Accordingly, the identification of small-molecule compounds that inhibit, regulate and/or modulate the signal transduction of kinases, such as Jak1, is a desirable means to prevent or treat autoimmune diseases or other diseases related to aberrant Jak1 function.

Jak2 is also activated in a wide variety of human cancers such as prostate, colon, ovarian and breast cancers, melanoma, leukemia and other hematopoietic malignancies. In addition, somatic point mutation of the Jak2 gene has been identified to be highly associated with classic myeloproliferative disorders (MPD) and infrequently in other myeloid disorders. Constitutive activation of Jak2 activity is also caused by chromosomal translocation in hematopoietic malignancies. It has also been shown that inhibition of the Jak/STAT pathway, and in particular inhibition of Jak2 activity, results in anti-proliferative and pro-apoptotic effects largely due to inhibition of phosphorylation of STAT. Furthermore, pharmacological modulation or inhibition of Jak2 activity could effectively block tumor growth and induce apoptosis by reducing the STAT phosphorylation in cell culture and human tumor xenografts in vivo. Accordingly, the identification of small-molecule compounds that inhibit, regulate and/or modulate the signal transduction of kinases, particularly Jak2, is desirable as a means to treat or prevent diseases and conditions associated with cancers.

Jak kinases also transmit signals regulating essential physiological processes whose inhibition could be undesirable. For example Jak2 mediates the signaling of Erythropoietin (Epo) and Granulocyte/Monocyte-Colony Stimulating Factor (GM-CSF). Individuals with genetic, congenital or acquired defects in these signaling pathways can develop potentially life-threatening complications such as anemia and neutrophil dysfunction. Accordingly, one non-limiting aspect of this invention also relates to a method to identify compounds that may have a favorable safety profile as a result of them selectively avoiding inhibition of Jak2.

Spleen tyrosine kinase (Syk) (J. Bio. Chem, 1991, 266, 15790) is a non-receptor tyrosine kinase that plays a key role in immunoreceptor signaling in a host of inflammatory cells including B cells, mast cells, macrophages and neutrophils. Syk is related to zeta associated protein 70 (ZAP-70) but also demonstrates similarity with JAK, Src and Tec family kinases.

Syk plays a critical and specific role in B-cell receptor (BCR) signaling on auto-reactive B cells and in FcR signaling on mast cells, macrophages, osteoclasts and neutrophils. (see Immunology Today, 2002, 21(3), 148 and Current Opinion in Immunology 2002, 14(3), 341).

Syk plays a key role in the activation mediated by Fc receptors of sentinel cells (mast cells and macrophages) and effector cells (neutrophils, basophils and eosinophils). The importance of Syk in rheumatoid arthritis is substantiated by data demonstrating the importance of Fc receptors (FcR) function and immune complexes in disease pathogenesis. Syk also mediates the activation of B cells through the BCR, which results in their expansion and the production of antispecific immunoglobulins. Therefore any disease that revolves around antibody-Fc receptor interactions may be modulated by Syk suppression. Thus a Syk inhibitor is likely to dampen both the initiation of the disease by blocking BCR signaling and the effector phase of the disease by blocking FcR signaling on macrophages, neutrophils and mast cells. Furthermore, blocking Syk would provide the added benefit of inhibiting osteoclast maturation and therefore attenuate bony erosions, joint destruction and generalized osteopenia associated with rheumatoid arthritis. Moreover Syk acts upstream close to the receptors at the initiation of complex signaling events and thus its inhibition influences all responses elicited by the activating agent. In mast cells for example, inhibition of Syk blocks the early release of a number of granule contents, as well as the subsequent production and secretion of lipid mediators and cytokines. Syk inhibitors can thus impart multiple beneficial effects as each of these mediators play distinct roles in the integrated inflammatory response.

The protein kinase C family is a group of serine/threonine kinases that comprises twelve related isoenzymes. Its members are encoded by different genes and are sub-classified according to their requirements for activation. The classical enzymes (cPKC) require diacylglycerol (DAG), phosphatidylserine (PS) and calcium for activation. The novel PKC's (nPKC) require DAG and PS but are calcium independent. The atypical PKC's (aPKC) do not require calcium or DAG.

PKCtheta is a member of the nPKC sub-family (Baier, G., et al., J. Biol. Chem., 1993, 268, 4997). It has a restricted expression pattern, found predominantly in T cells and skeletal muscle (Mischak, H. et al., FEBS Lett., 1993, 326, p. 51), with some expression reported in mast cells (Liu, Y. et al., J. Leukoc. Biol., 2001, 69, p. 831) and endothelial cells (Mattila, P. et al., Life Sci., 1994, 55, p. 1253).

Upon T cell activation, a supramolecular activation complex (SMAC) forms at the site of contact between the T cell and the antigen presenting cell (APC). PKCtheta is the only PKC isoform found to localize at the SMAC (Monks, C. et al., Nature, 1997, 385, 83), placing it in proximity with other signaling enzymes that mediate T cell activation processes.

In another study (Baier-Bitterlich, G. et al., Mol. Cell. Biol., 1996, 16, 842) the role of PKCtheta in the activation of AP-1, a transcription factor important in the activation of the IL-2 gene, was confirmed. In unstimulated T cells, constitutively active PKCtheta stimulated AP-1 activity while in cells with dominant negative PKCtheta, AP-1 activity was not induced upon activation by PMA.

Other studies showed that PKCtheta, via activation of IκB kinase beta, mediates activation of NF-κB induced by T cell receptor/CD28 co-stimulation (N. Coudronniere et al., Proc. Nat. Acad. Sci. U.S.A., 2000, 97, p. 3394; and Lin, X. et al., Mol. Cell. Biol., 2000, 20, p. 2933).

Proliferation of peripheral T cells from PKCtheta knockout mice, in response to T cell receptor (TCR)/CD28 stimulation was greatly diminished compared to T cells from wild type mice. In addition, the amount of IL-2 released from the T cells was also greatly reduced (Sun, Z. et al., Nature, 2000, 404, p. 402). It has also been shown that PKCtheta-deficient mice show impaired pulmonary inflammation and airway hyperresponsiveness (AHR) in a Th2-dependent murine asthma model, with no defects in viral clearance and Th1-dependent cytotoxic T cell function (Berg-Brown, N. N. et al., J. Exp. Med., 2004, 199, p. 743; Marsland, B. J. et al., J. Exp.

Med., 2004, 200, p. 181). The impaired Th2 cell response results in reduced levels of IL-4 and immunoglobulin E (IgE), contributing to the AHR and inflammatory pathophysiology. Otherwise, the PKCtheta knockout mice seemed normal and fertile.

Evidence also exists that PKCtheta participates in the IgE receptor (FcεRI)-mediated response of mast cells (Liu, Y. et al., J. Leukoc. Biol., 2001, 69, p. 831). In human-cultured mast cells (HCMC), it has been demonstrated that PKC kinase activity rapidly localizes to the membrane following FcεRI cross-linking (Kimata, M. et al., Biochem. Biophys. Res. Commun., 1999, 257(3), p. 895). A recent study examining in vitro activity of bone marrow mast cells (BMMC) derived from wild-type and PKCtheta-deficient mice shows that upon FceRI cross linking, BMMCs from PKCtheta-deficient mice reduced levels of IL-6, tumor necrosis factor-alpha (TNFα) and IL-13 in comparison with BMMCs from wild-type mice, suggesting a potential role for PKCtheta in mast cell cytokine production in addition to T cell activation (Ciarletta, A. B. et al., poster presentation at the 2005 American Thoracic Society International Conference).

The studies cited above and others studies confirm the critical role of PKCtheta in T cells activation and in mast cell (MC) signaling. Thus an inhibitor of PKCtheta would be of therapeutic benefit in treating immunological disorders and other diseases mediated by the inappropriate activation of T cells and MC signaling.

Many of the kinases, whether a receptor or non-receptor tyrosine kinase or a S/T kinase have been found to be involved in cellular signaling pathways involved in numerous pathogenic conditions, including immunomodulation, inflammation, or proliferative disorders such as cancer.

Many autoimmune diseases and disease associated with chronic inflammation, as well as acute responses, have been linked to excessive or unregulated production or activity of one or more cytokines.

The compounds of the invention are also useful in the treatment of cardiovascular disorders, such as acute myocardial infarction, acute coronary syndrome, chronic heart failure, myocardial infarction, atherosclerosis, viral myocarditis, cardiac allograft rejection, and sepsis-associated cardiac dysfunction. Furthermore, the compounds of the present invention are also useful for the treatment of central nervous system disorders such as meningococcal meningitis, Alzheimer's disease and Parkinson's disease.

The compounds of the invention are also useful in the treatment of an ocular condition, a cancer, a solid tumor, a sarcoma, fibrosarcoma, osteoma, melanoma, retinoblastoma, a rhabdomyosarcoma, glioblastoma, neuroblastoma, teratocarcinoma, hypersensitivity reactions, hyperkinetic movement disorders, hypersensitivity pneumonitis, hypertension, hypokinetic movement disorders, aortic and peripheral aneurysms, hypothalamic-pituitary-adrenal axis evaluation, aortic dissection, arterial hypertension, arteriosclerosis, arteriovenous fistula, ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, heart transplant rejection, hemachromatosis, hemodialysis, hemolytic uremic syndrome/thrombolytic thrombocytopenic purpura, hemorrhage, idiopathic pulmonary fibrosis, antibody mediated cytotoxicity, Asthenia, infantile spinal muscular atrophy, inflammation of the aorta, influenza A, ionizing radiation exposure, iridocyclitis/uveitis/optic neuritis, juvenile spinal muscular atrophy, lymphoma, myeloma, leukaemia, malignant ascites, hematopoietic cancers, a diabetic condition such as insulin-dependent diabetes mellitus glaucoma, diabetic retinopathy or microangiopathy, sickle cell anaemia, chronic inflammation, glomerulonephritis, graft rejection, Lyme disease, von Hippel Lindau disease, pemphigoid, Paget's disease, fibrosis, sarcoidosis, cirrhosis, thyroiditis, hyperviscosity syndrome, Osler-Weber-Rendu disease, chronic occlusive pulmonary disease, asthma or edema following burns, trauma, radiation, stroke, hypoxia, ischemia, ovarian hyperstimulation syndrome, post perfusion syndrome, post pump syndrome, post-MI cardiotomy syndrome, preeclampsia, menometrorrhagia, endometriosis, pulmonary hypertension, infantile hemangioma, or infection by Herpes simplex, Herpes Zoster, human immunodeficiency virus, parapoxvirus, protozoa or toxoplasmosis, progressive supranucleo palsy, primary pulmonary hypertension, radiation therapy, Raynaud's phenomenon, Raynaud's disease, Refsum's disease, regular narrow QRS tachycardia, renovascular hypertension, restrictive cardiomyopathy, sarcoma, senile chorea, senile dementia of Lewy body type, shock, skin allograft, skin changes syndrome, dry eye, ocular or macular edema, ocular neovascular disease, scleritis, radial keratotomy, uveitis, vitritis, myopia, optic pits, chronic retinal detachment, post-laser treatment complications, conjunctivitis, Stargardt's disease, Eales disease, retinopathy, macular degeneration, restenosis, ischemia/reperfusion injury, ischemic stroke, vascular occlusion, carotid obstructive disease, ulcerative colitis, inflammatory bowel disease, diabetes, diabetes mellitus, insulin dependent diabetes mellitus, allergic diseases, dermatitis scleroderma, graft versus host disease, organ transplant rejection (including but not limited to bone marrow and solid organ rejection), acute or chronic immune disease associated with organ transplantation, sarcoidosis, disseminated intravascular coagulation, Kawasaki's disease, nephrotic syndrome, chronic fatigue syndrome, Wegener's granulomatosis, Henoch-Schoenlein purpurea, microscopic vasculitis of the kidneys, chronic active hepatitis, septic shock, toxic shock syndrome, sepsis syndrome, cachexia, infectious diseases, parasitic diseases, acquired immunodeficiency syndrome, acute transverse myelitis, Huntington's chorea, stroke, primary biliary cirrhosis, hemolytic anemia, malignancies, Addison's disease, idiopathic Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, peripheral vascular disorders, peritonitis, pernicious anemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis A, Hepatitis B, Hepatitis C, His bundle arrythmias, HIV infection/HIV neuropathy, common varied immunodeficiency (common variable hypogammaglobulinaemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, pneumocystis carinii pneumonia, pneumonia, connective tissue disease associated interstitial lung disease, mixed connective tissue disease, associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthritis, primary sclerosing cholangitis, psoriasis type 1, psoriasis type 2, idiopathic leucopenia, autoimmune neutropenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, acute and chronic pain (different forms of pain), Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, toxicity, transplants, and diseases involving inappropriate vascularization for example diabetic retinopathy, retinopathy of prematurity, choroidal neovascularization due to age-related macular degeneration, and infantile hemangiomas in human beings. In addition, such compounds may be useful in the treatment of disorders such as ascites, effusions, and exudates, including for example macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, preeclampsia, menometrorrhagia, cat scratch fever, rubeosis, neovascular glaucoma and retinopathies such as those associated with diabetic retinopathy, retinopathy of prematurity, or age-related macular degeneration. In addition, these compounds can be used as active agents against hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

Compounds of Formula (I) of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the compound of the present invention. The additional agent also can be an agent that imparts a beneficial attribute to the therapeutic composition e.g., an agent that affects the viscosity of the composition.

It should further be understood that the combinations which are to be included within this invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

Preferred combinations are non-steroidal anti-inflammatory drug(s) also referred to as NSAIDS which include drugs like ibuprofen. Other preferred combinations are corticosteroids including prednisolone; the well known side-effects of steroid use can be reduced or even eliminated by tapering the steroid dose required when treating patients in combination with the compounds of this invention. Non-limiting examples of therapeutic agents for rheumatoid arthritis with which a compound of Formula (I) of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, and PDGF. Compounds of the invention can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7 (U.S. Pat. No. 6,090,382, HUMIRA™), CA2 (REMICADE™), SIMPONI™ (golimumab), CIMZIA™, ACTEMRA™, CDP 571, and soluble p55 or p75 TNF receptors, derivatives, thereof, (p75TNFR1gG (ENBREL™) or p55TNFR1gG (Lenercept), and also TNFα converting enzyme (TACE) inhibitors; similarly IL-1 inhibitors (Interleukin-1-converting enzyme inhibitors, IL-1RA etc.) may be effective for the same reason. Other preferred combinations include Interleukin 11. Yet other preferred combinations are the other key players of the autoimmune response which may act parallel to, dependent on or in concert with IL-18 function; especially preferred are IL-12 antagonists including IL-12 antibodies or soluble IL-12 receptors, or IL-12 binding proteins. It has been shown that IL-12 and IL-18 have overlapping but distinct functions and a combination of antagonists to both may be most effective. Yet another preferred combination is non-depleting anti-CD4 inhibitors. Yet other preferred combinations include antagonists of the co-stimulatory pathway CD80 (B7.1) or CD86 (B7.2) including antibodies, soluble receptors or antagonistic ligands.

A compound of Formula (I) of the invention may also be combined with agents, such as methotrexate, 6-mercaptopurine, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g., NIK, IKK, p38 or MAP kinase inhibitors), IL-1β converting enzyme inhibitors, T-cell signaling inhibitors such as kinase inhibitors, metalloproteinase inhibitors, sulfasalazine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™) and p55TNFRIgG (Lenercept), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl hydrocodone bitartrate/apap, diclofenac sodium/misoprostol, fentanyl, anakinra, tramadol HCl salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL15, BIRB-796, SC10-469, VX-702, AMG-548, VX-740, Roflumilast, IC-485, CDC-801, S1P1 agonists (such as FTY720), PKC family inhibitors (such as Ruboxistaurin or AEB-071) and Mesopram. Preferred combinations include methotrexate or leflunomide and in moderate or severe rheumatoid arthritis cases, cyclosporine and anti-TNF antibodies as noted above.

Non-limiting examples of therapeutic agents for inflammatory bowel disease with which a compound of Formula (I) of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, IL-23, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signaling by proinflammatory cytokines such as TNFα or IL-1 (e.g. NIK, IKK, or MAP kinase inhibitors); IL-1β converting enzyme inhibitors; TNFα converting enzyme inhibitors; T-cell signaling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-11, IL-13 and TGFβ). Preferred examples of therapeutic agents for Crohn's disease with which a compound of Formula (I) can be combined include the following: TNF antagonists, for example, anti-TNF antibodies, D2E7 (U.S. Pat. No. 6,090,382, HUMIRA™), CA2 (REMICADE™), CDP 571, TNFR-Ig constructs, (p75TNFRIgG (ENBREL™) and p55TNFRIgG (LENERCEPT™) inhibitors and PDE4 inhibitors. A compound of Formula (I) can be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors; 6-mercaptopurine; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-gamma.

Non-limiting examples of therapeutic agents for multiple sclerosis with which a compound of Formula (I) can be combined include the following: corticosteroids; prednisolone; methylprednisolone; azathioprine; cyclophosphamide; cyclosporine; methotrexate; 4-aminopyridine; tizanidine; interferon-β1a (AVONEX®; Biogen); interferon-β3b (BETASERON®; Chiron/Berlex); interferon α-n3) (Interferon Sciences/Fujimoto), interferon-α (Alfa Wassermann/)&J), interferon β1A-IF (Serono/Inhale Therapeutics), Peginterferon α 2b (Enzon/Schering-Plough), Copolymer 1 (Cop-1; COPAXONE®; Teva Pharmaceutical Industries, Inc.); hyperbaric oxygen; intravenous immunoglobulin; cladribine; antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of Formula (I) can be combined with antibodies to cell surface molecules such as CD2, CD3, CD4, CD8, CD19, CD20, CD25, CD28, CD30, CD40, CD45, CD69, CD80, CD86, CD90 or their ligands. A compound of Formula (I) may also be combined with agents such as methotrexate, cyclosporine, FK506, rapamycin, mycophenolate mofetil, leflunomide, an S1P1 agonist, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g., NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

Preferred examples of therapeutic agents for multiple sclerosis in which a compound of Formula (I) can be combined to include interferon-β, for example, IFNβ1a and IFNβ1b; copaxone, corticosteroids, caspase inhibitors, for example inhibitors of caspase-1, IL-1 inhibitors,

TNF inhibitors, and antibodies to CD40 ligand and CD80.

A compound of Formula (I) may also be combined with agents, such as alemtuzumab, dronabinol, daclizumab, mitoxantrone, xaliproden hydrochloride, fampridine, glatiramer acetate, natalizumab, sinnabidol, α-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL-4 agonists.

Non-limiting examples of therapeutic agents for ankylosing spondylitis with which a compound of Formula (I) can be combined include the following: ibuprofen, diclofenac, misoprostol, naproxen, meloxicam, indomethacin, diclofenac, celecoxib, rofecoxib, sulfasalazine, methotrexate, azathioprine, minocyclin, prednisone, and anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™) Non-limiting examples of therapeutic agents for asthma with which a compound of

Formula (I) can be combined include the following: albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin HCl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine HCl/pseudoephed, phenylephrine/cod/promethazine, codeine/promethazine, cefprozil, dexamethas one, guaifenesin/pseudoephedrine, chlorpheniramine/hydrocodone, nedocromil sodium, terbutaline sulfate, epinephrine, methylprednisolone, anti-IL-13 antibody, and metaproterenol sulfate.

Non-limiting examples of therapeutic agents for COPD with which a compound of Formula (I) can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol HCl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, cilomilast and roflumilast.

Non-limiting examples of therapeutic agents for HCV with which a compound of Formula (I) can be combined include the following: Interferon-alpha-2α, Interferon-alpha-2β, Interferon-alpha con1, Interferon-alpha-n1, pegylated interferon-alpha-2α, pegylated interferon-alpha-2β, ribavirin, peginterferon alfa-2b+ribavirin, ursodeoxycholic acid, glycyrrhizic acid, thymalfasin, Maxamine, VX-497 and any compounds that are used to treat HCV through intervention with the following targets: HCV polymerase, HCV protease, HCV helicase, and HCV IRES (internal ribosome entry site).

Non-limiting examples of therapeutic agents for Idiopathic Pulmonary Fibrosis with which a compound of Formula (I) can be combined include the following: prednisone, azathioprine, albuterol, colchicine, albuterol sulfate, digoxin, gamma interferon, methylprednisolone sodium succinate, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone HCl, potassium chloride, triamcinolone acetonide, tacrolimus anhydrous, calcium, interferon-alpha, methotrexate, mycophenolate mofetil and interferon-gamma-1β.

Non-limiting examples of therapeutic agents for myocardial infarction with which a compound of Formula (I) can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril hydrochloride/magnesium carbonate, bumetanide, alteplase, enalaprilat, amiodarone hydrochloride, tirofiban HCl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine HCl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, and cariporide.

Non-limiting examples of therapeutic agents for psoriasis with which a compound of Formula (I) can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine, ABT-874 and ustekinamab.

Non-limiting examples of therapeutic agents for psoriatic arthritis with which a compound of Formula (I) can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, D2E7 (U.S. Pat. No. 6,090,382, HUMIRA™), and efalizumab.

Non-limiting examples of therapeutic agents for restenosis with which a compound of Formula (I) can be combined include the following: sirolimus, paclitaxel, everolimus, tacrolimus,

ABT-578, and acetaminophen.

Non-limiting examples of therapeutic agents for sciatica with which a compound of Formula (I) can be combined include the following: hydrocodone bitartrate/apap, rofecoxib, cyclobenzaprine HCl, methylprednisolone, naproxen, ibuprofen, oxycodone HCl/acetaminophen, celecoxib, valdecoxib, methylprednisolone acetate, prednisone, codeine phosphate/apap, tramadol HCl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone HCl, tizanidine HC, diclofenac sodium/misoprostol, propoxyphene n-pap, asa/oxycod/oxycodone ter, ibuprofen/hydrocodone bit, tramadol HCl, etodolac, propoxyphene HCl, amitriptyline HCl, carisoprodol/codeine phos/asa, morphine sulfate, multivitamins, naproxen sodium, orphenadrine citrate, and temazepam.

Preferred examples of therapeutic agents for SLE (Lupus) with which a compound of Formula (I) can be combined include the following: NSAIDS, for example, diclofenac, naproxen, ibuprofen, piroxicam, indomethacin; COX2 inhibitors, for example, celecoxib, rofecoxib, valdecoxib; anti-malarials, for example, hydroxychloroquine; steroids, for example, prednisone, prednisolone, budenoside, dexamethasone; cytotoxics, for example, azathioprine, cyclophosphamide, mycophenolate mofetil, methotrexate; inhibitors of PDE4 or purine synthesis inhibitor, for example Cellcept®. A compound of Formula (I) may also be combined with agents such as sulfasalazine, 5-aminosalicylic acid, olsalazine, Imuran® and agents which interfere with synthesis, production or action of proinflammatory cytokines such as IL-1, for example, caspase inhibitors like IL-1β converting enzyme inhibitors and IL-1ra. A compound of Formula (I) may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of Formula (I) can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of Formula (I) may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™).

In this invention, the following definitions are applicable:

A “therapeutically effective amount” is an amount of a compound of Formula (I) or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art.

“Pharmaceutically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g. (+) or (−)-tartaric acid or mixtures thereof), amino acids (e.g. (+) or (−)-amino acids or mixtures thereof), and the like. These salts can be prepared by methods known to those skilled in the art.

Certain compounds of Formula (I) which have acidic substituents may exist as salts with pharmaceutically acceptable bases. The present invention includes such salts. Examples of such salts include sodium salts, potassium salts, lysine salts and arginine salts. These salts may be prepared by methods known to those skilled in the art.

Certain compounds of Formula (I) and their salts may exist in more than one crystal form and the present invention includes each crystal form and mixtures thereof.

Certain compounds of Formula (I) and their salts may also exist in the form of solvates, for example hydrates, and the present invention includes each solvate and mixtures thereof. Certain compounds of Formula (I) may contain one or more chiral centers, and exist in different optically active forms. When compounds of Formula (I) contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of Formula (I) contains more than one chiral center, it may exist in diastereoisomeric forms. The diastereoisomeric compounds may be separated by methods known to those skilled in the art, for example chromatography or crystallization and the individual enantiomers may be separated as described above. The present invention includes each diastereoisomer of compounds of Formula (I) or Formula (II), and mixtures thereof.

Certain compounds of Formula (I) may exist in different tautomeric forms or as different geometric isomers, and the present invention includes each tautomer and/or geometric isomer of compounds of Formula (I) and mixtures thereof.

Certain compounds of Formula (I) may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of Formula (I) and mixtures thereof.

Certain compounds of Formula (I) may exist in zwitterionic form and the present invention includes each zwitterionic form of compounds of Formula (I) and mixtures thereof.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The pro-drug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial.

Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents wherein the free hydrogen is replaced by (C₁-C₄)alkyl, (C₁-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of Formula (I) wherein the free hydrogen of the hydroxyl substituent (e.g., R group contains hydroxyl) is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₁₂)alkoxycarbonyloxymethyl, N—(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, P(O)(OH)₂, —P(O)(O(C₁-C₆)alkyl)₂ or glycosyl (the radical resulting from detachment of the hydroxyl of the hemiacetal of a carbohydrate).

The term “heterocyclic”, “heterocyclyl” or “heterocyclylene”, as used herein, include non-aromatic, ring systems, including, but not limited to, monocyclic, bicyclic, tricyclic and spirocyclic rings, which can be completely saturated or which can contain one or more units of unsaturation, for the avoidance of doubt, the degree of unsaturation does not result in an aromatic ring system) and have 5 to 12 atoms including at least one heteroatom, such as nitrogen, oxygen, or sulfur. For purposes of exemplification, which should not be construed as limiting the scope of this invention, the following are examples of heterocyclic rings: azepinyl, azetidinyl, indolinyl, isoindolinyl, morpholinyl, piperazinyl, piperidinyl, pyrrolidinyl, quinucludinyl, thiomorpholinyl, tetrahydropyranyl, tetrahydrofuranyl, tetrahydroindolyl, thiomorpholinyl and tropanyl.

The term “heteroaryl” or “heteroarylene” as used herein, include aromatic ring systems, including, but not limited to, monocyclic, bicyclic and tricyclic rings, and have 5 to 12 atoms including at least one heteroatom, such as nitrogen, oxygen, or sulfur. For purposes of exemplification, which should not be construed as limiting the scope of this invention: azaindolyl, benzo(b)thienyl, benzimidazolyl, benzofuranyl, benzoxazolyl, benzothiazolyl, benzothiadiazolyl, benzoxadiazolyl, furanyl, imidazolyl, imidazopyridinyl, indolyl, indazolyl, isoxazolyl, isothiazolyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrazolyl, thiadiazolyl, or thienyl.

As used herein, “alkyl”, “alkylene” or notations such as “(C₁-C₈)” include straight chained or branched hydrocarbons which are completely saturated. Examples of alkyls are methyl, ethyl, propyl, isopropyl, butyl, pentyl, hexyl and isomers thereof. As used herein, “alkenyl”, “alkenylene”, “alkynylene” and “alkynyl” means C₂-C₈ and includes straight chained or branched hydrocarbons which contain one or more units of unsaturation, one or more double bonds for alkenyl and one or more triple bonds for alkynyl.

As used herein, “aromatic” groups (or “aryl” or “arylene” groups) include aromatic carbocyclic ring systems (e.g. phenyl) and fused polycyclic aromatic ring systems (e.g. naphthyl, biphenyl and 1,2,3,4-tetrahydronaphthyl).

As used herein, “cycloalkyl” or “cycloalkylene” means C₃-C₁₂ monocyclic or multicyclic (e.g., bicyclic, tricyclic, spirocyclic, etc.) hydrocarbons that are completely saturated or have one or more unsaturated bonds but does not amount to an aromatic group. Examples of a cycloalkyl group are cyclopropyl, cyclobutyl, cyclopentyl, cyclopentenyl, cyclohexyl and cyclohexenyl.

As used herein, many moieties or substituents are termed as being either “substituted” or “optionally substituted”. When a moiety is modified by one of these terms, unless otherwise noted, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, —C(O)—(C₁-C₃)alkylene-O-optionally substituted aryl, —C(O)-optionally substituted (C₃-C₆)cycloalkyl, —NHC(O)H, —NHC(O) (C₁-C₈)alkyl groups, —NHC(O) (C₃-C₈)cycloalkyl groups, —N((C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, —N((C₁-C₈)alkyl)C(O)NH₂ groups, —NHC(O)N((C₁-C₈)alkyl)₂ groups, —N((C₁-C₈)alkyl)C(O)N((C₁-C₈)alkyl)₂ groups, —N((C₁-C₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, —N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S— halogenated (C₁-C₈)alkyl groups (for example but not limited to —SCF₃), —(C₁-C₆) heterocyclyl (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

“ [symbol] ” in Formula (I) represents an aromatic ring.

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few hours up to over several days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition of a given protein kinase activity). In some cases it is appropriate to determine the IC₅₀ in the presence of 3 to 5% serum albumin since such a determination approximates the binding effects of plasma protein on the compound. Such information can be used to more accurately determine useful doses in humans. Further, the most preferred compounds for systemic administration effectively inhibit protein kinase signaling in intact cells at levels that are safely achievable in plasma.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition (see e.g. Fingl et al., 1975, in “The Pharmacological Basis of Therapeutics”, Ch. 1 p. 1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition.

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the following Examples.

### a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

### b) Tablets

Tablets can be prepared, for example, from the following ingredients.

Parts by Weight

| | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

### c) Enteric Coated Tablets

Tablets can be prepared by the method described in (b) above. The tablets can be enteric coated in a conventional manner using a solution of 20% cellulose acetate phthalate and 3% diethyl phthalate in ethanol: dichloromethane (1:1).

d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

In the compositions of the present invention the active compound may, if desired, be associated with other compatible pharmacologically active ingredients. For example, the compounds of this invention can be administered in combination with another therapeutic agent that is known to treat a disease or condition described herein. For example, with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate. The additional pharmaceutical agents include, but are not limited to, anti-edemic steroids, NSAIDS, ras inhibitors, anti-TNF agents, anti-IL1 agents, antihistamines, PAF-antagonists, COX-1 inhibitors, COX-2 inhibitors, NO synthase inhibitors, Akt/PTB inhibitors, IGF-1R inhibitors, PKC inhibitors, PI3 kinase inhibitors, calcineurin inhibitors and immunosuppressants. The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deleterious effects of a hyperproliferative disorder, angiogenesis, vascular hyperpermeability or edema than the administration of either substance alone. In the treatment of malignant disorders combinations with antiproliferative or cytotoxic chemotherapies or radiation are included in the scope of the present invention.

The present invention also comprises the use of a compound of Formula (I) as a medicament.

A further aspect of the present invention provides the use of a compound of Formula (I) or a salt thereof in the manufacture of a medicament for treating vascular hyperpermeability, angiogenesis-dependent disorders, proliferative diseases and/or disorders of the immune system in mammals, particularly human beings.

The present invention also provides a method of treating vascular hyperpermeability, inappropriate neovascularization, proliferative diseases and/or disorders of the immune system which comprises the administration of a therapeutically effective amount of a compound of Formula (I) to a mammal, particularly a human being, in need thereof.

### ABBREVIATIONS

AcOH Glacial acetic acid
BSA Bovine serum albumin
BuOH Butanol
d Doublet
dd Doublet of doublets
dba Dibenzylideneacetone
DCE Dichloroethane
DCM Dichloromethane (methylene chloride)
DEA Diethylamine
DIEA N,N-Diisopropylethylamine
DMEM Dulbecco's Modified Eagle Medium
DMF N,N-Dimethylformamide
DMSO Dimethyl sulfoxide
DNP-HSA Dinitrophenyl-human serum albumin
dppf 1,1′-Bis(diphenylphosphino)ferrocene
EDC N-(3-Dimethylaminopropyl)-N′-ethylcarbodiimide
EDTA Ethylene diamine tetraacetic acid
equiv Equivalent(s)
EtOAc Ethyl acetate
Et₂O Diethyl ether
EtOH Ethanol
FBS Fetal bovine serum
FLAG DYKDDDDK peptide sequence
g Gram(s)
GST Glutathione S-transferase
h Hour(s)
HPLC High-pressure liquid chromatography
Hz Hertz
i.d. Intradermal
IFA Incomplete Freunds Adjuvant
i-Pr Isopropyl
KOAc Potassium acetate
LC Liquid chromatography
m Multiplet
M Molar
MeCN Acetonitrile
MeOH Methyl alcohol
min Minute(s)
mL Milliliter(s)
mmol Millimole
MOPS 3-(N-morpholino)-propanesulfonic acid
MOPSO 3-(N-morpholino)-2-hydroxypropanesulfonic acid
MS Mass spectrometry
N Normal
NBS N-Bromosuccinimide
NH₄OAc Ammonium acetate
NMR Nuclear magnetic resonance
or Optical rotation
OVA Ovalbumin
PBS Phosphate buffered saline
PFPAA 2,2,3,3,3-Pentafluoropropanoic Anhydride
pH -log[H⁺]
pNAG Nitrophenyl-N-acetyl-β-D-glucosaminide
PPh₃ Triphenylphosphine
ppm Parts per million
psi Pounds per square inch
rcf Relative centrifugal force
Rₜ Retention time
rt Room temperature
s Singlet
SEM 2-(Trimethylsilyl)ethoxymethyl
SFC Supercritical Fluid Chromatography
t Triplet
t- Tertiary
TBAF Tetra-n-butylammonium fluoride
TEA Triethylamine
TFA Trifluoroacetic acid
THF Tetrahydrofuran
TLC Thin layer chromatography
USP United States Pharmacopeia
UV Ultraviolet
wt % Weight percent

### Assays

In Vitro Jak1 Kinase Activity Measured by Time-Resolved Fluorescence Resonance Energy Transfer (trFRET)

Varying concentrations of inhibitor were added to an assay well containing: Jak1 enzyme (aa 845-1142; expressed in SF9 cells as a GST fusion and purified by glutathione affinity chromatography; 4 nM), peptide substrate (biotin-TYR2, Sequence: Biotin-(Ahx)-AEEEYFFLFA-amide; 2 μM), MOPSO pH 6.5 (50 mM), MgCl₂ (10 mM), MnCl₂ (2 mM), DTT (2.5 mM), BSA (0.01% w/v), Na₃VO₄ (0.1 mM) and ATP (0.001 mM). After about 60 min incubation at rt, the reaction was quenched by addition of EDTA (final concentration: 100 mM) and developed by addition of revelation reagents (final approximate concentrations: 30 mM HEPES pH 7.0, 0.06% BSA, 0.006% Tween-20, 0.24 M KF, 80 ng/mL PT66K (europium labeled anti-phosphotyrosine antibody cat #61T66KLB Cisbio, Bedford, Mass.) and 3.12 μg/mL SAXL (Phycolink streptavidin-allophycocyanin acceptor, cat #PJ52S, Prozyme, San Leandro, Calif.). The developed reaction was incubated in the dark either at about 4° C. for about 14 h or for about 60 min at rt, then read via a time-resolved fluorescence detector (Rubystar, BMG) using a 337 nm laser for excitation and emission wavelength of 665 nm. Within the linear range of the assay, the observed signal at 665 nm is directly related to phosphorylated product and used to calculate the IC₅₀ values.

In Vitro Jak3 Kinase Activity Measured by Time-Resolved Fluorescence Resonance Energy Transfer (trFRET)

Varying concentrations of inhibitor were added to an assay well containing: Jak3 enzyme (aa 811-1103; expressed in SF9 cells as a GST fusion and purified by glutathione affinity chromatography; 3 nM), peptide substrate (biotin-TYR2, Sequence: Biotin-(Ahx)-AEEEYFFLFA-amide; 2 μM), MOPSO pH 6.5 (50 mM), MgCl₂ (10 mM), MnCl₂ (2 mM), DTT (2.5 mM), BSA (0.01% w/v), Na₃VO₄ (0.1 mM) and ATP (0.001 mM). After about 60 min incubation at rt, the reaction was quenched by addition of EDTA (final concentration: 100 mM) and developed by addition of revelation reagents (final approximate concentrations: 30 mM HEPES pH 7.0, 0.06% BSA, 0.006% Tween-20, 0.24 M KF, 80 ng/mL PT66K (europium labeled anti-phosphotyrosine antibody cat #61T66KLB Cisbio, Bedford, Mass.) and 0.8 μg/mL SAXL (Phycolink streptavidin-allophycocyanin acceptor, cat #PJ52S, Prozyme, San Leandro, Calif.). The developed reaction was incubated in the dark either at about 4° C. for about 14 h or for about 60 min at rt, then read via a time-resolved fluorescence detector (Rubystar, BMG) using a 337 nm laser for excitation and emission wavelength of 665 nm. Within the linear range of the assay, the observed signal at 665 rim is directly related to phosphorylated product and used to calculate the IC₅₀ values.

In Vitro Syk Kinase Activity Measured by Time-Resolved Fluorescence Resonance Energy Transfer (trFRET)

0.3 nM Syk catalytic domain (aa356-635, purified in-house at the Abbott Bioresearch Center) was mixed with 0.1 μM peptide substrate (biotin-TYR1, Sequence: Biotin-(Ahx)-GAEEEIYAAFFA-COOH) at varying concentrations of inhibitor in reaction buffer: 50 mM MOPSO pH 6.5, 10 mM MgCl₂, 2 mM MnCl₂, 2.5 mM DTT, 0.01% BSA, 0.1 mM Na₃VO₄ and 0.001 mM ATP. After about 60 min incubation at rt, the reaction was quenched by addition of EDTA (final concentration: 100 mM) and developed by addition of revelation reagents (final approximate concentrations: 30 mM HEPES pH 7.0, 0.06% BSA, 0.006% Tween-20, 0.24 M KF, 90 ng/mL PT66K (europium labeled anti-phosphotyrosine antibody cat #61T66KLB Cisbio, Bedford, Mass.) and 0.6 μg/mL SAXL (Phycolink streptavidin-allophycocyanin acceptor, cat #PJ52S, Prozyme, San Leandro, Calif.). The developed reaction was incubated in the dark either at about 4° C. for about 14 h or for about 60 min at rt, then read via a time-resolved fluorescence detector (Rubystar, BMG) using a 337 nm laser for excitation and emission wavelength of 665 nm. Within the linear range of the assay, the observed signal at 665 nm is directly related to phosphorylated product and used to calculate the IC₅₀ values.

Other In Vitro Kinase Assays Measured by Time-Resolved Fluorescence Resonance Energy Transfer (trFRET)

Other kinase assays were performed using a similar protocol. Additional purified enzymes Tyk2 (aa 880-1185 with an N-terminal histidine-tag and C-terminal FLAG tag; purified in-house by immobilized metal ion affinity chromatography), RET (aa 711-1072 with an N-terminal histidine-tag; purified by immobilized metal ion affinity chromatography), Syk (aa356-635 with a C-terminal histidine tag; purified by immobilized metal ion affinity chromatography), and KDR (aa 792-1354 with an N-terminal histidine-tag; purified in-house by immobilized metal ion affinity and ion-exchange chromatography) were expressed in SF9 cells and Aurora 1/B (aa1-344 with a N-terminal histidine-tag and purified by immobilized metal ion affinity chromatography) was expressed in E. coli. Other enzymes used are available from commercial sources. Enzymes were mixed with biotinylated substrates at varying concentrations of inhibitor in different reaction buffers (see Table A). After about 60 min incubation at rt, the reaction was quenched by addition of EDTA and developed by addition of revelation reagents (final approximate concentrations: 30 mM HEPES pH 7.0, 0.06% BSA, 0.006% Tween-20, 0.24 M KF, varying amounts of donor europium labeled antibodies and acceptor streptavidin labeled allophycocyanin (SAXL)). The developed reactions were incubated in the dark either at about 4° C. for about 14 h or for about 60 min at rt, then read in a time-resolved fluorescence detector (Rubystar, BMG Labtech) as described above.

TABLE A
Specific conditions (per 40 μL enzyme reaction) for the various enzymes are
detailed below:

| | | | | Enzyme | | | ATP | DMSO | Reaction | |
|---|---|---|---|---|---|---|---|---|---|---|
| | | | Assay | Conc. | Substrate | | Conc. | Conc. | Time | Detection |
| Enzyme | Construct | Substrate | Buffer | (ng/well) | Conc. | | (mM) | (%) | (min) | condition |
| Jak1 | aa 845- | Biotin- | MOPSO | 5 | 2 | μM | 0.001 | 5 | 60 | 8 ng/well |
| | 1142 | TYR2 | | | | | | | | PT66K, |
| | | | | | | | | | | 0.39 |
| | | | | | | | | | | μg/well |
| | | | | | | | | | | SAXL |
| Jak2 | Millipore | Biotin- | MOPSO | 2.5 | 2 | μM | 0.001 | 5 | 60 | 8 ng/well |
| | cat# 14-640 | TYR1 | | | | | | | | PT66K, |
| | | | | | | | | | | 0.078 |
| | | | | | | | | | | μg/well |
| | | | | | | | | | | SAXL |
| Jak3 | aa 811- | Biotin- | MOPSO | 4.5 | 2 | μM | 0.001 | 5 | 60 | 8 |
| | 1103 | TYR2 | | | | | | | | ng/well |
| | | | | | | | | | | PT66K, |
| | | | | | | | | | | 0.078 |
| | | | | | | | | | | μg/well |
| | | | | | | | | | | SAXL |
| Tyk2 | aa880- | Biotin- | MOPSO | 9 | 2 | μM | 0.001 | 5 | 60 | 8 |
| | 1185 | TYR1 | | | | | | | | ng/well |
| | | | | | | | | | | PT66K, |
| | | | | | | | | | | 0.078 |
| | | | | | | | | | | μg/well |
| | | | | | | | | | | SAXL |
| Aurora | aa1-344 | KinEAS | MOPS | 20 | 0.5 | μM | 0.1 | 5 | 60 | 15 |
| 1/B | | E S2 | | | | | | | | ng/well |
| | | | | | | | | | | Eu- |
| | | | | | | | | | | STK- |
| | | | | | | | | | | Ab, |
| | | | | | | | | | | 0.34 |
| | | | | | | | | | | μg/wel |
| | | | | | | | | | | SAXL |
| KDR | aa789-1354 | Biotin- | HEPES | 10 | 2 | μM | 0.1 | 5 | 60 | 8 ng/well |
| | | TYR2 | | | | | | | | PT66K, |
| | | | | | | | | | | 0.078 |
| | | | | | | | | | | μg/well |
| | | | | | | | | | | SAXL |
| JNK1 | Millipore | Biotin | MOPS | 10 | 1 | μM | 0.01 | 5 | 60 | 2.58 |
| | cat# 14-327 | ATF2- | | | | | | | | ng/well |
| | | pep | | | | | | | | Anti- |
| | | | | | | | | | | pATF2- |
| | | | | | | | | | | Eu, |
| | | | | | | | | | | 0.6 |
| | | | | | | | | | | μg/well |
| | | | | | | | | | | SAXL |
| JNK2 | Millipore | Biotin- | MOPS | 5 | 0.5 | μM | 0.01 | 5 | 60 | 2.58 |
| | cat# 14-329 | ATF2- | | | | | | | | ng/well |
| | | pep | | | | | | | | Anti- |
| | | | | | | | | | | pATF2- |
| | | | | | | | | | | Eu, |
| | | | | | | | | | | 0.6 |
| | | | | | | | | | | μg/well |
| | | | | | | | | | | SAXL |
| RET | aa711-1072 | Biotin- | HEPES | 4 | 10 | ng/well | 0.01 | 5 | 60 | 8 ng/well |
| | | poly | | | | | | | | PT66K, |
| | | GluTyr | | | | | | | | 0.078 |
| | | | | | | | | | | μg/well |
| | | | | | | | | | | SAXL |
| P70 S6 | Millipore | KinEAS | MOPS | 0.5 | 0.25 | μM | 0.01 | 5 | 60 | 15 |
| Kinase | cal # 14- | E S3 | | | | | | | | ng/well |
| | 486 | | | | | | | | | Eu-STK- |
| | | | | | | | | | | Ab, |
| | | | | | | | | | | 0.34 |
| | | | | | | | | | | μg/well |
| | | | | | | | | | | SAXL |
| PKN2 | Invitrogen | KinEAS | MOPS | 0.7 | 0.5 | μM | 0.001 | 5 | 60 | 15 |
| | cat # | E S3 | | | | | | | | ng/well |
| | PV3879 | | | | | | | | | Eu-STK- |
| | | | | | | | | | | Ab, |
| | | | | | | | | | | 0.34 |
| | | | | | | | | | | μg/well |
| | | | | | | | | | | SAXL |
| Syk | aa356-635 | Biotin- | MOPSO | 0.4 | 0.1 | μM | 0.001 | 5 | 60 | 6.8 |
| | | TYR1 | | | | | | | | ng/well |
| | | | | | | | | | | PT66K, |
| | | | | | | | | | | 0.045 |
| | | | | | | | | | | μg/well |
| | | | | | | | | | | SAXL |
| CDK2/ | Millipore | Biotin- | MOPS | 50 | 2 | μM | 0.1 | 5 | 60 | 15 |
| Cyclin | cat # 14- | MBP | | | | | | | | ng/well |
| A | 448 | | | | | | | | | Anti- |
| | | | | | | | | | | pMBP- |
| | | | | | | | | | | Eu; 0.34 |
| | | | | | | | | | | μg/well |
| | | | | | | | | | | SAXL |

### Reaction Buffers:

MOPSO buffer contains: 50 mM MOPSO pH 6.5, 10 mM MgCl₂, 2 mM MnCl₂, 2.5 mM DTT, 0.01% BSA, and 0.1 mM Na₃VO₄

HEPES buffer contains: 50 mM HEPES pH 7.1, 2.5 mM DTT, 10 mM MgCl₂, 2 mM MnCl₂, 0.01% BSA, and 0.1 mM Na₃VO₄

MOPS buffer contains: 20 mM MOPS pH 7.2, 10 mM MgCl₂, 5 mM EGTA, 5 mM Beta-phosphoglycerol, 1 mM Na₃VO₄, 0.01% Triton-X-100 and 1 mM DTT

### Substrates:

Biotin-ATF2-peptide sequence: Biotin-(Ahx)-AGAGDQTPTPTRFLKRPR-amide
Biotin-TYR1-peptide sequence: Biotin-(Ahx)-GAEEEIYAAFFA-COOH
Biotin-TYR2-peptide sequence: Biotin-(Ahx)-AEEEYFFLFA-amide
Biotin-MBP-peptide sequence: Biotin-(Ahx)-VHFFKNIVTPRTPPPSQGKGAEGQR-amide
Biotin-polyGluTyr peptide was purchased from Cisbio (cat #61 GT0BLA, Bedford, Mass.)
KinEASE S2 and S3 peptides were purchased from Cisbio (cat #62ST0PEB, Bedford, Mass.)

### Detection Reagents:

Anti-pATF2-Eu was custom-labeled by Cisbio (Bedford, Mass.)
Anti-pMBP-Eu was custom-labeled by Cisbio (Bedford, Mass.)
PT66K was purchased from Cisbio (cat #61T66KLB, Bedford, Mass.)
SAXL was purchased from Prozyme (cat #PJ25S, San Leandro, Calif.)
   Human T-Blasts IL-2 pSTAT5 Cellular Assay

### Materials:

Phytohemaglutinin T-blasts were prepared from Leukopacks purchased from Biological Specialty Corporation, Colmar, Pa. 18915, and cryopreserved in 5% DMSO/media prior to assay.

For this assay the cells were thawed in assay medium with the following composition: RPMI 1640 medium (Gibco 11875093) with 2 mM L-glutamine (Gibco 25030-081), 10 mM HEPES (Gibco 15630-080), 100 μg/mL Pen/Strep (Gibco 15140-122), and 10% heat inactivated FBS (Gibco 10438026). Other materials used in the assay: DMSO (Sigma D2650), 96-well dilution plates (polypropylene) (Corning 3365), 96-well assay plates (white, ½ area, 96 well) (Corning 3642), D-PBS (Gibco 14040133), IL-2 (R&D 202-IL-10 (10 μg)), Alphascreen pSTAT5 kit (Perkin Elmer TGRS5S10K) and Alphascreen protein A kit (Perkin Elmer 6760617M)

### Methods:

T-Blasts were thawed and cultured for about 24 h without IL-2 prior to assay. Test compounds or controls are dissolved and serially diluted in 100% DMSO. DMSO stocks are subsequently diluted 1:50 in cell culture media to create the 4× compound stocks (containing 2% DMSO). Using a Corning white 96 well, ½ area plate, cells are plated at 2×10⁵/10 μl/well in 10 μL media followed by addition of 5 μL of 4× test compound in duplicate. Cells are incubated with compound for about 0.5 h at about 37° C. Next, 5 μL of IL-2 stock is added at 20 ng/mL final concentration. IL-2 is stored as a 4 μg/mL stock solution, as specified by the manufacturer, at about ˜20° C. in aliquots and diluted 1:50 with assay media (to 80 ng/mL) just prior to use. The contents of the wells are mixed by carefully tapping sides of plate(s) several times followed by incubation at about 37° C. for about 15 min. The assay is terminated by adding 5 μL of 5× AlphaScreen lysis buffer and shaking on an orbital shaker for about 10 min at rt. Alphascreen acceptor bead mix is reconstituted following Perkin Elmer's protocol. 30 μL/well of reconstituted Alphascreen acceptor bead mix was added, covered with foil then shaken on orbital shaker for about 2 min on high then about 2 h on low. Donor bead mix is reconstituted following Perkin Elmer's AlphaScreen protocol; 12 μL/well are added, covered with foil then shaken for about 2 min on high, and about 2 h on low. Plates are read on an EnVision reader following Perkin Elmer's AlphaScreen protocol instructions.

TF-1 IL-6 pSTAT3 Cellular Assay

### Materials:

TF-1 cells (ATCC #CRL-2003). Culture medium: DMEM medium (Gibco 11960-044) with 2 mM L-glutamine (Gibco 25030-081), 10 mM HEPES (Gibco 15630-080), 100 μg/mL Pen/Strep (Gibco 15140-122), 1.5 g/L sodium bicarbonate (Gibco 25080-094), 1 mM sodium pyruvate (Gibco 11360-070), 10% heat inactivated FBS (Gibco 10437-028), and 2 ng/mL GM-CSF (R&D 215-GM-010). Other materials used in this assay: DMSO (Sigma D2650), 96-well dilution plates (polypropylene) (Corning 3365), 96-well assay plates (white, ½ area, 96 well) (Corning 3642), D-PBS (Gibco 14040133), IL-6 (R&D 206-IL/CF-050 (50 μg)), Alphascreen pSTAT3 kit (Perkin Elmer TGRS3S10K) and Alphascreen protein A kit (Perkin Elmer 6760617M).

### Methods:

Prior to the assay, cells are cultured for about 18 h in the culture medium without GM-CSF. Test compounds or controls are dissolved and serially diluted in 100% DMSO. DMSO stocks are subsequently diluted 1:50 in cell culture media to create the 4× compound stocks (containing 2% DMSO). Using a Corning white 96 well, ½ area plate, cells are plated at 2×10⁷/10 μL/well in 10 μL media followed by addition of 5 μL of the 4× test compound stock in duplicate. Cells are incubated with compound for about 0.5 h at about 37° C. followed by addition of 5 μL of 400 ng/mL IL-6. IL-6 is stored in 10 μg/mL aliquots using endotoxin free D-PBS (0.1% BSA) at about −20° C. Prior to assay IL-6 is diluted to 400 ng/mL in culture media and applied (5 μL/well) to all wells, except to negative control wells where 5 μL/well of media is added. The contents of the wells are mixed carefully by tapping the side of the plate several times. Plates are incubated at about 37° C. for about 30 min. Cells are lysed by adding 5 μL of 5× AlphaScreen cell lysis buffer to all wells, shaken for about 10 min at rt then assayed. Alternatively, assay plates may be frozen at about −80° C. and thawed later at rt. Using the pSTAT3 SureFire Assay kit (Perkin Elmer #TGRS3S10K) acceptor bead mix is reconstituted following Perkin Elmer's AlphaScreen protocol instructions. 30 μL are added per well then the plate is covered with foil and shaken on an orbital shaker for about 2 min on high, then about 2 h on low at rt. Donor bead mix is reconstituted following Perkin Elmer's AlphaScreen protocol instructions. 12 μL are added per well, then covered with foil and shaken on orbital shaker for about 2 min on high, then about 2 h on low at about 37° C. Plates are read on an EnVision reader following Perkin Elmer's AlphaScreen protocol instructions at rt.

UT7/EPO pSTAT5 Cellular Assay

### Materials:

UT7/EPO cells are passaged with erythropoietin (EPO), split twice per week and fresh culture medium is thawed and added at time of split. Culture Medium: DMEM medium (Gibco 11960-044) with 2 mM L-glutamine (Gibco 25030-081), 10 mM HEPES (Gibco 15630-080), 100 U/mL Pen/Strep (Gibco 15140-122), 10% heat inactivated FBS (Gibco 10437-028), EPO (5 μL/mL=7.1 μL of a 7 μg/mL stock per mL of medium). Assay media: DMEM, 2 mM L-glutamine, 5% FBS, 10 mM HEPES. Other materials used in the assay: DMSO (Sigma D2650), 96-well dilution plates (polypropylene) (Corning 3365), 96-well assay plates (white, ½ area, 96 well) (Corning 3642), D-PBS (Gibco 14040133), IL-2 (R&D 202-IL-10 (10 μg)), Alphascreen pSTAT5 kit (Perkin Elmer TGRS5S10K) and Alphascreen protein A kit (Perkin Elmer 6760617M).

### Methods:

Culture cells for about 16 h without EPO prior to running assay. Test compounds or controls are dissolved and serially diluted in 100% DMSO. DMSO stocks are subsequently diluted 1:50 in cell culture media to create the 4× compound stocks (containing 2% DMSO). Using a Corning white 96 well, ½ area plate, cells are plated at 2×10⁵/10 μL/well in 10 μL media followed by addition of 5 μL of 4× test compound stock in duplicate. Cells are incubated with compound for about 0.5 h at about 37° C. After incubation, 5 μL of EPO is added to afford a final concentration of 1 nM EPO. The contents of the wells are mixed by carefully tapping sides of the plate several times followed by incubation at about 37° C. for about 20 min. 5 μL of 5× AlphaScreen lysis buffer are added followed by shaking on an orbital shaker for about 10 min at rt. 30 μL/well of acceptor beads are added after reconstitution following Perkin Elmer's AlphaScreen protocol, covered with foil and shaken on orbital shaker for about 2 min on high, then about 2 h on low. Donor beads are reconstituted following Perkin Elmer's AlphaScreen protocol instructions followed by addition of 12 μL/well, covered with foil and shaken on an orbital shaker for about 2 min on high, about 2 h on low. Plates are read on an EnVision reader following Perkin Elmer's AlphaScreen protocol instructions.

### Antigen-Induced Degranulation of RBL-2H3 Cells:

RBL-2H3 cells are maintained in T75 flasks at about 37° C. and 5% CO₂, and passaged every 3-4 days. To harvest cells, 20 mL of PBS is used to rinse the flask once, and then 3 mL of Trypsin-EDTA is added and incubated at about 37° C. for about 2 min. Cells are transferred to a tube with 20 mL medium, spun down at 1000 RPM at rt for about 5 min and resuspended at 1×10⁶ cells/mL. Cells are sensitized by adding DNP-specific mouse IgE to a final concentration of 0.1 μg/mL. 50 μL of cells are added to each well of a 96 well flat bottom plate (50×10³ cells/well) and incubated overnight at about 37° C. in 5% CO₂. The next day, compounds are prepared in 100% DMSO at 10 mM. Each compound is then serially diluted 1:4 six times in 100% DMSO. Each compound dilution is then diluted 1:20 and then 1:25, both dilutions in Tyrode's buffer. Media is aspirated from the cell plates and the cells are rinsed twice with 100 μL of Tyrode's buffer (prewarmed to about 37° C.). 50 μL of compounds diluted in Tyrode's buffer are added to each well and the plates are incubated for about 15 min at about 37° C. in 5% CO₂. 50 μL of 0.2 μg/mL DNP-HSA in Tyrode's buffer is then added to each well and the plates are incubated for about 30 min at about 37° C. in 5% CO₂. The final concentration of the various components in the incubation mix are 0.002-10 μM compounds, 0.1% DMSO, and 0.1 μg/mL DNP-HSA. As one control, 0.2% DMSO (no compound) in Tyrode's buffer is added to a set of wells to determine maximum stimulated release. As a second control, Tyrode's buffer without DNP-HSA is added to a set of wells with containing 0.2% DMSO without compounds to determine unstimulated release. Each condition (compounds and controls) is set up in triplicate wells. At the end of the 30 min incubation, 50 μL of supernate is transferred to a new 96 well plate. The remaining supernate in the cell plates is aspirated and replaced with 50 μL of 0.1% Triton X-100 in Tyrode's buffer to lyse the cells. 50 μL of freshly prepared 1.8 mM 4-Nitrophenyl N-acetyl-β-D-glucosaminide (pNAG) is then added to each well of supernate and cell lysate and the plates are incubated for about 60 min at about 37° C. in 5% CO₂. 100 μL of 7.5 mg/mL sodium bicarbonate is added to each well to stop the reaction. The plates are then read at 405 nm on a Molecular Devices SpectraMax 250 plate reader.

### Calculation of Results

1) The plate background OD₄₀₅ obtained from wells containing Tyrode's buffer and pNAG (no supernate or lysate) is subtracted from the OD₄₀₅ reading for each well containing supernate or lysate.
2) The release for each well is expressed as the percentage of the total release for that well, where the total release is twice the release in the supernate plus the release in the cell lysate. This calculation corrects for variable cell number in each well.
3) The maximum response is the mean response of wells containing DNP-HSA but no compound.
4) The minimum response is the mean response of wells containing no DNP-HSA and no compound.

5) The response in each compound well is calculated as a percentage of the maximum response (expressed as % control) where the maximum response is 100% and the minimum response is 0%.
6) A dose response curve is generated for each compound and the IC₅₀ of the curve is calculated using Prism GraphPad software and nonlinear least squares regression analysis.

### Acute In Vivo Measurement of JAK Inhibition by Compounds is Measured Using the:

### Concanavalin A (Con A)-Induced Cytokine Production in Lewis Rats

The test compound is formulated in an inert vehicle (for example but not limited to 0.5% hydroxypropylmethyl cellulose (Sigma, cat #H3785)/0.02% Tween 80 (Sigma, cat #4780) in water) at the desired concentration to achieve doses in the range of 0.01-100 mg/kg. Six-week-old male Lewis rats (125 g-15 0 g) (Charles River Laboratories) are dosed with the compound orally, at time zero (0 min) After about 30 min the rats are injected intravenously (i.v.) with 10 mg/kg Concanavalin A (Con A, AmershamBioscience, cat #17-0450-01) dissolved in PBS (Invitrogen, cat #14190). About 4 h later, the rats are cardiac bled and their plasma is analyzed for levels of IL-2 (ELISA kit: R&D Systems cat #R2000) and IFN-γ (ELISA kit: R&D Systems cat #RIF00).

### Acute In Vivo Measurement of Fcγ Receptor Signaling Inhibition of the Compounds is Measured Using the:

### Reverse Passive Arthus Model

On day 0, OVA was made up at a concentration of 17.5 mg/mL, in PBS by rocking gently until a solution was formed. 2% Evans Blue solution (Sigma Aldrich, cat #E2129) was then added to double the volume for a final concentration of 8.75 mg/mL of OVA and 1% Evans Blue dye. Anti-OVA antibody (Abazyme), stock concentration 10 mg/mL, was thawed and a 400 μg/100 μL solution was made with PBS. Compounds were made up by adding the vehicle, 0.5% HPMC with 0.02% Tween80, and vortexing for about 15 seconds followed by homogenizing for a minimum of about 2 min at 28,000 rpm until there was a fine particulate suspension with no clumps of compound. Rats were weighed and dosed with compound at a pre-determined t-max based on pharmacokinetic studies Animals were then placed under general anesthesia with a 5% isoflourane and oxygen mixture and shaved. Using a ½ mL insulin syringe two sites were injected i.d., 1 site with 100 μL of 400 μg/100 μL of anti-OVA antibody, and 1 site with 100 μL of sterile PBS. Each site was then circled with permanent marker for explant later. Right after i.d. injections animals were injected with 200 μL of the OVA (10mg/kg)/Evans Blue mixture i.v., using a ½ mL insulin syringe. About four hours post injection animals were euthanized, bled via cardiac puncture and blood was collected using a plasma separating tube. Blood samples were stored on ice until centrifugation (within about 2 h of collection). Each injection site was removed with a disposable biopsy punch (Acuderm Acu-Punch Disposable 12 mm), cut into four pieces and placed in a pre-labeled 2 mL eppendorf tube. One mL of DMF was added to each biopsy tube and placed in a heat block for about 24 h at about 50° C. About 24 h after incubation 100 μL of each sample was added to a 96 well flat bottom plate. The samples were read at 620 nm on a plate reader using the Softmax software in order to measure the levels of Evan's Blue dye. Background was removed by subtracting the OD from the PBS injected site from the OD of the anti-OVA injected site for each individual animal. Plasma samples were spun down in a microcentrifuge for about 5 min at 16.1 ref. 200 μL of plasma was placed in a 1.7 mL eppendorf tube for drug level measurement and tubes were stored at -80° C. until evaluation.

### Chronic In Vivo Effects of the Compounds on an Arthritis Disease Model is Measured Using the:

Adjuvant Induced Arthritis (AIA) model in a Lewis Rat

Female Lewis rats, (6 weeks of age, 125 g-150 g in weight from Charles River Laboratories) are immunized intradermally (i.d.) in the right hind-footpad with 100 μL of a suspension of mineral oil (Sigma, cat #M5905) and containing 200 μg M. tuberculosis, H37RA (Difco, cat #231141). The inflammation appears in the contra-lateral (left) hind paw seven days after the initial immunization. Seven days post immunization, the compound is formulated in an inert vehicle (for example but not limited to 0.5% hydroxypropylmethyl cellulose (Sigma, cat #H3785)/0.02% Tween 80 (Sigma, cat #4780) in water) and dosed orally once or twice a day for at least 10 days. Baseline paw volume is taken on day 0 using a water displacement pleythsmograph (Vgo Basile North America Inc. PA 19473, Model #7140). Rats are lightly anesthetized with an inhalant anesthetic (isoflurane) and the contra-lateral (left) hind paw is dipped into the plethysmograph and the paw volume is recorded. The rats are scored every other day up to day 17 after immunization. On day 17 after immunization, all rats are exsanguinated by cardiac puncture under isoflurane anesthesia, and the left hind paw is collected to assess the impact on bone erosion using micro-CT scans (SCANCO Medical, Southeastern, PA, Model #μCT 40) at a voxel size of 18 μm, a threshold of 400, sigma-gauss 0.8, support-gauss 1.0. Bone volume and density is determined for a 360 μm (200 slice) vertical section encompassing the tarsal section of the paw. The 360 μm section is analyzed from the base of the metatarsals to the top of the tibia, with the lower reference point fixed at the tibiotalar junction. Drug exposure is determined in the plasma using LC/MS or the:

### Collagen Induced Arthritis (CIA) Model in a Lewis Rat

On day—1 Collagen Type II (CII), soluble from bovine nasal septum (Elastin Products, Cat #CN276) was weighed out for a dose of 600 μg/rat, 0.01M acetic acid (150 μL AcOH USP grade. J. T. Baker, order #9522-03, and 250 mL Milli Q Water) was added for a concentration of 4 mg/mL. The vial was covered with aluminum foil and placed on a rocker at about 4° C. overnight. On day 0 collagen stock solution was diluted 1:1 with Incomplete Freunds adjuvant (IFA) (Difco labs, cat #263910) using a glass Hamilton luer lock syringe (SGE Syringe Perfection VWR cat #007230), final concentration 2 mg/mL. Female Lewis rats (Charles River Laboratories) acclimated for 7 days at the time of immunization weighing approximately 150 g were anesthetized in an anesthesia chamber using isoflurane (5%) and oxygen. Once the rats were completely anesthetized, they were transferred to a nose cone to maintain anesthesia during the injections. Rats were shaved at the base of the tail, 300 μL of collagen was injected i.d. on the rump of the rat, n=9 per group. 100 μL at three sites with a 500 μL leur lock syringe and a 27 g needle. IFA control rats are injected in the same manner (n=6). The IFA is a 1:1 emulsion with the 0.01M acetic acid. Boost was done on day 6 of the study. Shaving was not done on this day and injections were done in the same manner as the immunization. The inflammation appears in both hind paws 10 days after the initial immunization. 10 days post immunization, the compound was formulated in an inert vehicle (for example but not limited to 0.5% hydroxypropylmethyl cellulose (Sigma, cat #H3785)/0.02% Tween 80 (Sigma, cat #4780) in water) and dosed orally once or twice a day for at least 9 days. Baseline paw volume was taken on day 7 using a water displacement pleythsmograph (Vgo Basile North America Inc. PA 19473, Model #7140). Rats were lightly anesthetized with an inhalant anesthetic (isoflurane) and both hind paws were dipped into the plethysmograph and the paw volume was recorded. The rats were scored 2 to 3 times a week up to day 18 after immunization. On day 18 after immunization, all rats were exsanguinated by cardiac puncture under isoflurane anesthesia, and the hind paws were collected to assess the impact on bone erosion using micro-CT scans (SCANCO Medical, Southeastern, PA, Model #μCT 40) at a voxel size of 18 μm, a threshold of 400, sigma-gauss 0.8, support-gauss 1.0. Bone volume and density was determined for a 360 μm (200 slice) vertical section encompassing the tarsal section of the paw. The 360 μm section was analyzed from the base of the metatarsals to the top of the tibia, with the lower reference point fixed at the tibiotalar junction. Drug exposure was determined from plasma using LC/MS.

### Chronic In Vivo Effects of the Compounds on an Asthma Disease Model is Measured Using the:

### OVA Induced Rat Asthma Model

Female Brown Norway rats (7-9 weeks of age) were sensitized on day 0 and 7 with 40 μg ovalbumin (OVA) (Sigma-Aldrich, St. Louis, Mo.) in a 20 mg/mL solution of Alum Imject (Pierce, Rockford, Ill.). The rats were subsequently challenged intratracheally on day 19 and 20 with 1.5 μg OVA in 50 μL PBS. Dosing of inhibitor began on day 18 and continues through day 22. On day 22, 48 h after the second challenge, rats were subjected to an anesthetized and restrained pulmonary function test. Airway hyperresponsiveness (AHR) was assessed using whole body plethysmography. Briefly, a surgical plane of anesthesia was induced with an intraperitoneal injection of 60 mg/kg ketamine and 5 mg/kg xylazine (Henry Schein, Inc., Melville, N.Y.). A tracheal cannula was surgically inserted between the 3rd and 4th tracheal rings. Spontaneous breathing was prevented by jugular vein injection of 0.12 mg/kg pancuronium bromide (Sigma-Aldrich, St Louis, Mo.). Animals were placed in a whole body plethysmograph (Buxco Electronics, Inc., Wilmington, N.C.) and mechanically ventilated with 0.2 mL room air at 150 breaths per minute with a volume controlled ventilator (Harvard Apparatus, Framingham, Mass.). Pressure in the lung and flow within the plethysmograph were measured using transducers and lung resistance was calculated as pressure/flow using Biosystem Xa software (Buxco Electronics). Airway resistance was measured at baseline and following challenge with 3, 10, and 30 mg/mL methacholine (Sigma Aldrich, St. Louis, Mo.) delivered with an inline ultrasonic nebulizer. Upon completion of pulmonary function testing, the lungs were lavaged 3 times with 1 mL sterile PBS. The volume from the first wash was centrifuged at 2000 rpm for 5 min, and the supernatant is stored for subsequent analysis. The volume of washes 2 through 3 are added to the pellet derived from the first wash and subsequently processed for evaluation of cellular infiltrate by flow cytometry. Plasma was collected from blood drawn from the vena cava and was used for evaluation of drug concentrations.

For the Examples below enzyme inhibition data is provided for Jak3 and/or Syk. The IC₅₀ value is expressed as follows:

A=a compound with an IC₅₀ less than 0.1 μM

B=a compound with an IC₅₀ within the range of 0.1 to 1.0 μM

C=a compound with an IC₅₀ within the range of 1.0 to 10.0 μM

D=a compound with an IC₅₀ greater than 10 μM.

The teachings of all references, including journal articles, patents and published patent applications, are incorporated herein by reference in their entirety.

The following examples are for illustrative purposes and are not to be construed as limiting the scope of the present invention.

### General Synthetic Schemes

Compounds of the invention may be prepared using the synthetic transformations illustrated in Schemes I-VI. Starting materials are commercially available, may be prepared by the procedures described herein, by literature procedures, or by procedures that would be well known to one skilled in the art of organic chemistry. Further fractionalization of any of the R groups in the Schemes below (e.g. R′, R″, R′″, R₁, R₂, R₅, and R₆) can be performed, if desired, at any point in the reaction sequence using reactions known to one skilled in the art (for example, Larock, R. C. “Comprehensive Organic Transformations: A Guide to Functional Group Preparations, 2nd edition”, 1999, Wiley-VCH). For example, formation of amides, ureas, sulfonamides, aryl amines, heteroaryl amines, sulfonyl ureas, substituted amines, or guanidines can be prepared with an R group containing a primary or secondary amine In a second non-limiting example, an R group containing a halide may be reacted with an amine to give a substituted amine. Also, deprotection of an R group to yield deprotected compounds may be performed using conditions such as those described in Greene, T. W. and Wuts, P. G. M. “Protective Groups in Organic Synthesis, 3rd Edition”, 1999, Wiley-Interscience and the deprotected compounds may then be reacted further as described above. In addition, prodrug moieties may be introduced to the intermediates or final compounds described herein.

Methods for preparing 7H-imidazo[1,5-c]pyrrolo[3,2-e]pyrimidine compounds of the invention are illustrated in Scheme I. The 4-chloro-7H-pyrrolo[2,3-d]pyrimidines 1 can be obtained commercially or prepared by one skilled in the art (for example, Bioorg. & Med. Chem. Lett. 2002, 12, 2153-2157). In Scheme I, step a, 4-chloro-7H-pyrrolo[2,3-d]pyrimidines 1 are reacted with sodium iodide in aqueous HI to give 4-iodo-7H-pyrrolo[2,3-d]pyrimidines 2 using conditions such as those described in Example #1, Step A. As shown in Scheme I, step b, 4-iodo-7H-pyrrolo[2,3-d]pyrimidines 2 may be protected on the pyrrole nitrogen with a SEM group using conditions known in the literature (Greene, T. W. and Wuts, P. G. M. [referenced above]) or as described in Example #1, Step B. The SEM-protected 4-iodo-7H-pyrrolo[2,3-d]pyrimidines 3 are reacted with aldehydes 4 to give alcohols 5 using conditions such as those described in Example #1, Step C, Example #3, Step A, or Org Lett 2003, 5, 4289-4291 (Scheme I, step c). Oxidation of alcohols 5 to ketones 6 (Scheme I, step d) may be accomplished using conditions known in the literature (Greene, T. W. and Wuts, P. G. M. [referenced above]) or as described in Example #1, Step D and Example #3, Step B. Formamides 7 may be prepared from ketones 6 (Scheme I, step e) with the conditions described in Example #1, Step E. Cyclization to SEM-protected 7H-imidazo[1,5-c]pyrrolo[3,2-e]pyrimidines 8 is accomplished using a dehydrating reagent, such as POCl₃, as described in Example #1, Step F (Scheme I, step f). The protecting group can then be removed (Scheme I, step g) to give 7H-imidazo[1,5-c]pyrrolo[3,2-e]pyrimidines 9 using standard conditions (for example, Greene, T. W. and Wuts, P. G. M. [referenced above] or Example #1, Step G). Alternatively, 3-substituted-SEM-protected 7H-imidazo[1,5-c]pyrrolo[3,2-e]pyrimidines 10 may be prepared from ketones 6 (Scheme I, step h) with the conditions described in Example #3, Step C. The protecting group can then be removed (Scheme I, step g) to give 3-substituted-7H-imidazo[1,5-c]pyrrolo[3,2-e]pyrimidines 11 using standard conditions (for example, Greene, T. W. and Wuts, P. G. M. [referenced above] or Example #3, Step D).

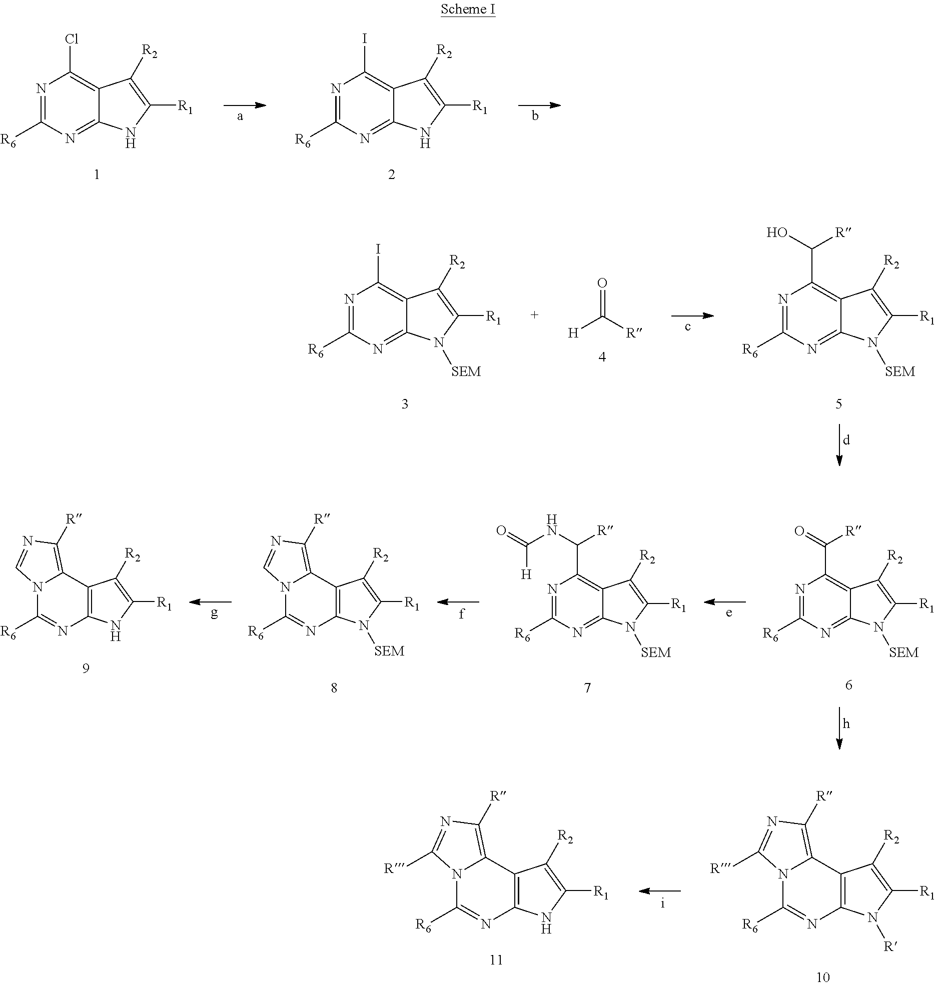

Methods for preparing 7H-pyrrolo[3,2-e][1,2,3]triazolo[1,5-c]pyrimidine compounds of the invention are illustrated in Scheme II. In Scheme II, step a, ketones 6 (from Scheme I, step d) are condensed with hydrazine to give hydrazones 12 using methods known to one skilled in the art (for example, Example #2, Step A or Larock, R. C. [referenced above]). The SEM-protected 7H-pyrrolo[3,2-e][1,2,3]triazolo[1,5-c]pyrimidines 13 are formed by iodobenzene diacetate mediated oxidation as described in Example#2, Step B or Syn. Comm. 2000, 30, 417-425 (Scheme II, step b). The protecting group can then be removed (Scheme II, step c) to give 7H-pyrrolo[3,2-e][1,2,3]triazolo[1,5-c]pyrimidines 14 using standard conditions (for example, Greene, T. W. and Wuts, P. G. M. [referenced above] or Example #2, Step C).

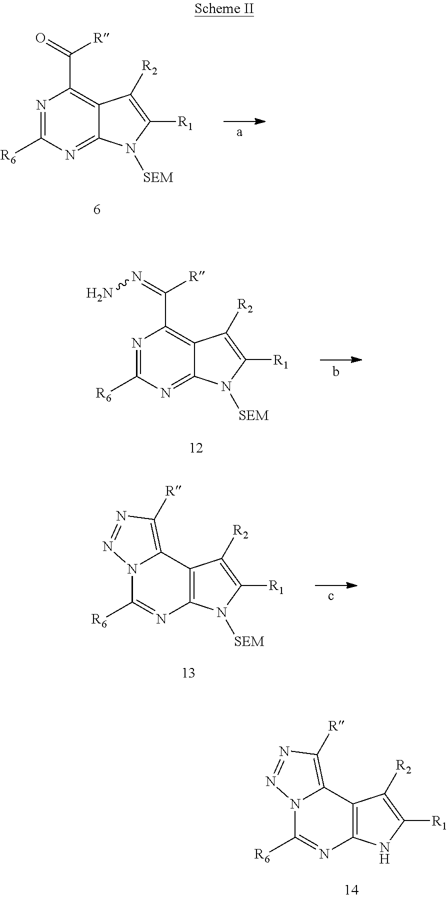

Methods for preparing 7H-pyrrolo[3,2-e][1,2,4]triazolo[4,3-c]pyrimidine compounds of the invention are illustrated in Scheme III. As shown in Scheme III, step a, 4-chloro-7H-pyrrolo[2,3-d]pyrimidines 1 can be protected on the pyrrole nitrogen with a SEM group using methods described in Example #7, Step B and Example #9, Step A, for instance, or by methods known to one skilled in the art (for example, Larock, R. C. [referenced above] or Greene, T. W. and Wuts, P. G. M. [referenced above]). The resulting 4-chloro-7-((2-(trimethylsilyl)ethoxy)-methyl)-7H-pyrrolo[2,3-d]pyrimidines 15 can then be reacted with hydrazine to give SEM-protected hydrazines 16 (Scheme III, step b) using methods known to one skilled in the art (for example, see Example #9, Step B). SEM-protected hydrazines 16 are then converted to SEM-protected 7H-pyrrolo[3,2-e][1,2,4]triazolo[4,3-c]pyrimidines 17 using the method described in Example #9, Step C (Scheme III, step c). The protecting group can then be removed (Scheme III, step d) to give 7H-pyrrolo[3,2-e][1,2,4]triazolo[4,3-c]pyrimidines 18 using standard conditions (for example, Greene, T. W. and Wuts, P. G. M. [referenced above] or Example #9, Step D). Alternatively, 4-chloro-7H-pyrrolo[2,3-d]pyrimidines 1 may be reacted directly with hydrazine hydrate (Scheme III, step e) as described in Example #8, Step A to give hydrazines 19 which are reacted with aldehydes 20 to give 7H-pyrrolo[3,2-e][1,2,4]triazolo[4,3-c]pyrimidines 18 as illustrated in Example #8, Step B (Scheme III, step f).

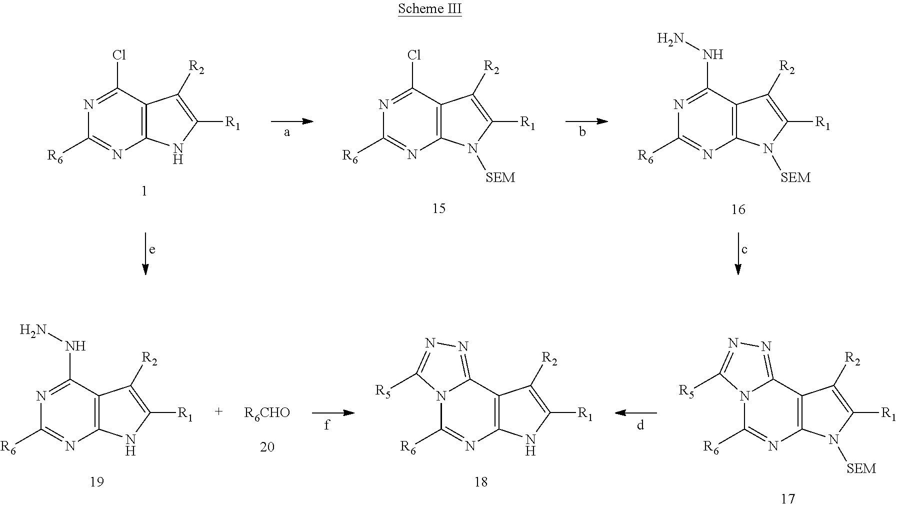

Methods for preparing 7H-imidazo[1,2-c]pyrrolo[3,2-e]pyrimidine compounds of the invention are illustrated in Scheme IV. In Scheme IV, the 4-chloro-7-((2-(trimethylsilyl)ethoxy)methyl)-7H-pyrrolo[2,3-d]pyrimidines 15 (prepared as described above in Scheme III, step a) can be converted to amines 21 using the conditions described in Example #7, Step C and Example #10, Step A (Scheme IV, step a). Reaction with a 2-chloroacetaldehyde using conditions described in Example #7, Step D and Example #10, Step B provides SEM-protected 7H-imidazo[1,2-c]pyrrolo[3,2-e]pyrimidines 22 (Scheme IV, step b). The protecting group can then be removed (Scheme IV, step c) to give 7H-imidazo[1,2-c]pyrrolo[3,2-e]pyrimidines 23 using standard conditions (for example, Greene, T. W. and Wuts, P. G. M. [referenced above] or Example #7, Step F).

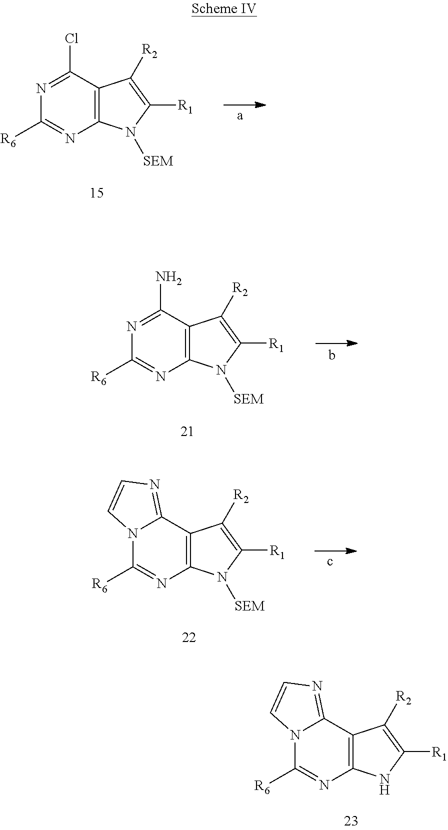

Additional methods for preparing 7H-imidazo[1,2-c]pyrrolo[3,2-e]pyrimidine compounds of the invention are illustrated in Scheme V. The SEM-protected 7H-imidazo[1,2-c]pyrrolo[3,2-e]pyrimidines 22 (prepared as described in Scheme IV, step b) can then be halogenated in the 3-position with a suitable halogenating agent (for example, see Example #10, Step C) to give heteroaryl halides 24 (Scheme V, step a). The 3-substituted-7H-imidazo[1,2-c]pyrrolo[3,2-e]pyrimidines 26 can then be obtained through deprotection of the SEM group (Scheme V, step b) using methods known to one skilled in the art (for example, see Example #10, Step D or Greene, T. W. and Wuts, P. G. M. [referenced above]) to give 3-halo-7H-imidazo[1,2-c]pyrrolo[3,2-e]pyrimidines 25 followed by a Suzuki coupling (for example, Example #10, Step F, or J. Organometallic Chem. 1999, 576, 147) with an aryl boronic acid or aryl boronate (Scheme V, step c). Alternatively, one could do the Suzuki coupling first to give 3-substituted-7-((2-(trimethylsilyl)ethoxy)methyl)-7H-imidazo[1,2-c]pyrrolo[3,2-e]pyrimidines 27 followed by deprotection as described above (Scheme V, steps d and e).

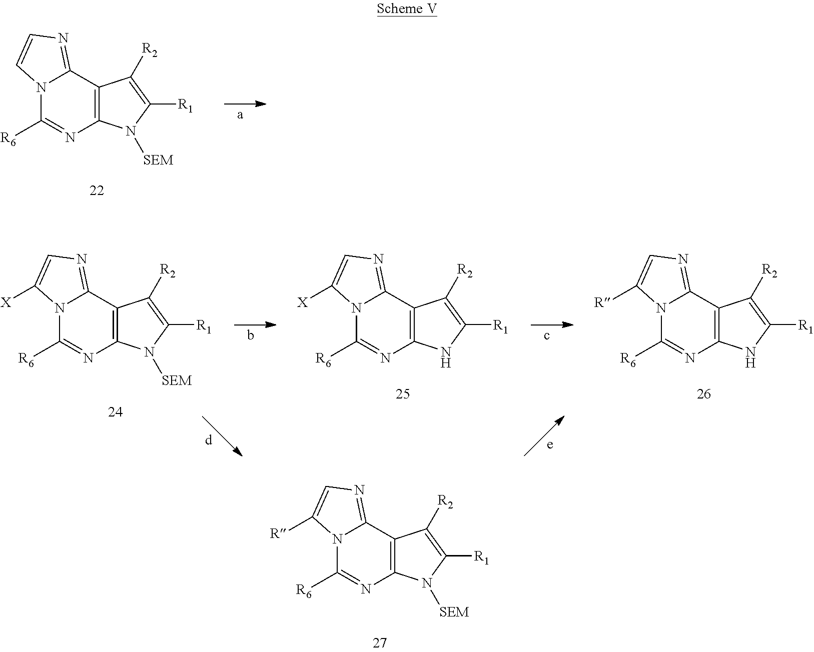

Additional methods for preparing 7H-imidazo[1,2-c]pyrrolo[3,2-e]pyrimidine compounds of the invention are illustrated in Scheme VI. Reaction of 7-((2-(trimethylsilyl)ethoxy)methyl)-7H-pyrrolo[2,3-d]pyrimidin-4-amines 21 (prepared as described in Scheme IV, step a) are reacted with α-bromoketones (for example, see Example #11, Step A) to give SEM-protected 2-substituted-7H-imidazo[1,2-c]pyrrolo[3,2-e]pyrimidines 28 (Scheme VI, step a). The protecting group can then be removed (Scheme VI, step b) to give 2-substituted-7H-imidazo[1,2-c]pyrrolo[3,2-e]pyrimidines 29 using standard conditions (for example, Greene, T. W. and Wuts, P. G. M. [referenced above] or Example #11, Step B).

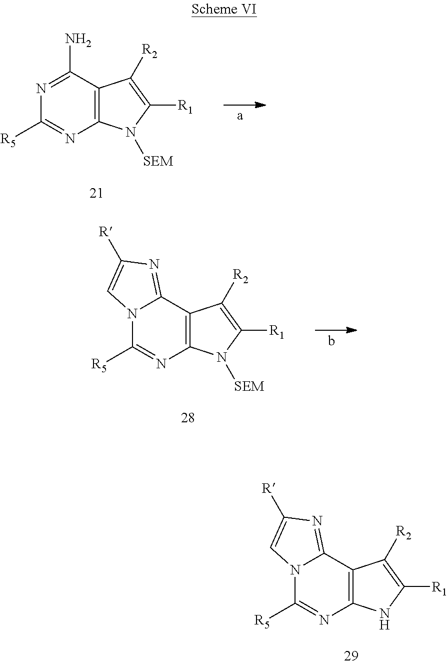

If desired, chiral separation of any of the chiral compounds in Schemes I-VI may be done using methods known to one skilled in the art such as chiral preparative HPLC or chiral SFC or crystallization of diastereomeric salts.

### Analytical Methods

Analytical data was included within the procedures below, in the illustrations of the general procedures, or in the tables of examples. Unless otherwise stated, all ¹H NMR data were collected on a Varian UNITY Plus 300 MHz, Varian Mercury 300 MHz, Varian Mercury Plus 300 or 400 MHz, or Varian Inova 600 MHz instrument and chemical shifts are quoted in parts per million (ppm). LC/MS and HPLC data are referenced to the table of LC/MS and HPLC conditions using the lower case method letter provided in Table 1.

TABLE 1
LC/MS and HPLC methods

| Method | Conditions |
|---|---|
| a | LC/MS: The gradient was 5-60% B in 1.5 min then 60-95% B to 2.5 min with a |
| | hold at 95% B for 1.2 min (1.3 mL/min flow rate). Mobile phase A was 10 mM |
| | NH₄OAc, mobile phase B was HPLC grade MeCN. The column used for the |
| | chromatography is a 4.6 × 50 mm MAC-MOD Halo C18 column (2.7 μm particles). |
| | Detection methods are diode array (DAD) and evaporative light scattering (ELSD) |
| | detection as well as positive/negative electrospray ionization. |
| b | LC/MS: The gradient was 0-0.1 min 10% A, 0.1-2.6 min 10-100% A, 2.6-2.9 min |
| | 100% A, 2.9-3.0 min 100-10% A then 0.5 min post-run delay. Flow rate was 2 mL/min. |
| | Mobile phase A was HPLC grade MeCN and mobile phase B was 0.1% |
| | TFA in water. The column used for the chromatography was a Phenomenex Luna |
| | Combi-HTS C8(2) 5 μm 100 Å (2.1 mm × 50 mm) at a temperature of 55° C. |
| | Detection methods were diode array (DAD) and evaporative light scattering (ELSD) |
| | detection as well as positive APCI ionization. |
| c | HPLC: Hypersil HS C18 column, 250 mm × 21.2 mm, 8 μm particle size, flow rate |
| | 21 mL/min, detection 254 nm, mobile phase A was 0.05N NH₄OAc pH 4.5 buffer, |
| | mobile phase B was MeCN, 5-100% B over 25 min. |
| d | LC/MS: The gradient was 5-60% B in 1.5 min then 60-95% B to 2.5 min with a |
| | hold at 95% B for 1.2 min (1.3 mL/min flow rate). Mobile phase A was 10 mM |
| | NH₄OAc, mobile phase B was HPLC grade MeCN. The column used for the |
| | chromatography was a 4.6 × 50 mm MAC-MOD Halo C8 column (2.7 μm particles). |
| | Detection methods were diode array (DAD) and evaporative light scattering (ELSD) |
| | detection as well as positive/negative electrospray ionization. |
| e | LC/MS: The gradient was 5-60% B in 0.75 min then 60-95% B to 1.15 min with a |
| | hold at 95% B for 0.75 min (1.3 mL/min flow rate). Mobile phase A was 10 mM |
| | NH₄OAc, mobile phase B was HPLC grade MeCN. The column used for the |
| | chromatography was a 4.6 × 50 mm MAC-MOD Halo C8 column (2.7 μm particles). |
| | Detection methods were diode array (DAD) and evaporative light scattering (ELSD) |
| | detection as well as positive/negative electrospray ionization. |
| f | HPLC: Hypersil HS C18 column, 250 mm × 21.2 mm, 8 μm particle size, flow rate |
| | 21 mL/min, detection 254 nm, mobile phase A was 0.05N NH₄OAc pH 4.5 buffer, |
| | mobile phase B was HPLC grade MeCN, 10-100% B over 25 min. |
| g | Chiral HPLC: Isocratic 40% A for 15-25 min (20 mL/min flow rate). Mobile phase |
| | A was EtOH (200 proof), mobile phase B was HPLC grade heptane with 0.1% DEA |
| | added. The column used for the chromatography was a Daicel IA, 20 × 250 mm |
| | column (5 μm particles). Detection methods were evaporative light scattering |
| | (ELSD) detection, and/or UV (variable wavelength) as well as optical rotation. |
| h | HPLC: Hypersil HS C18 column, 250 mm × 21.2 mm, 8 μm particle size, flow rate |
| | 21 mL/min, detection 254 nm, mobile phase A was 0.05N NH₄OAc pH 4.5 buffer, |
| | mobile phase B was MeCN, 10-100% B over 20 min. |
| i | HPLC: Hypersil HS C18 column, 250 mm × 21.2 mm, 8 μm particle size, flow rate |
| | 21 mL/min, detection 254 nm, mobile phase A was 0.05N NH₄OAc pH 4.5 buffer, |
| | mobile phase B was MeCN, 10-70% B over 20 min. |

### PREPARATIONS AND EXAMPLES

None of the specific conditions and reagents noted herein are to be construed as limiting the scope of the invention and are provided for illustrative purposes only. All starting materials are commercially available from Sigma-Aldrich (including Fluka and Discovery CPR) unless otherwise noted after the chemical name. Reagent/reactant names given are as named on the commercial bottle or as generated by IUPAC conventions, CambridgeSoft® ChemDraw Ultra 9.0.7, CambridgeSoft® Chemistry E-Notebook 9.0.127, or AutoNom 2000. Compounds designated as salts (e.g. hydrochloride, acetate) may contain more than one molar equivalent of the salt. Compounds of the invention where the absolute stereochemistry has been determined by the use of a commercially available enantiomerically pure starting material or a stereochemically defined intermediate, or by X-ray diffraction are denoted by an asterisk after the example number. Otherwise, stereochemistry is randomly assigned.

### Example #1

### 1-Cyclohexyl-7H-imidazo[1,5-c]pyrrolo[3,2-e]pyrimidine

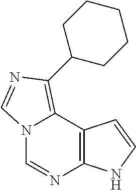

### Step A: 4-Iodo-7H-pyrrolo[2,3-d]pyrimidine

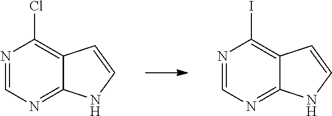

A mixture of 4-chloro-7H-pyrrolo[2,3-d]pyrimidine (10.0 g, 65.1 mmol) and sodium iodide (13.1 g, 87.0 mmol) in HI (57% stabilized in water) (43.0 mL, 326 mmol) was heated at about 60° C. The reaction was cooled to rt after about 16 h and then poured over a stirring ice/50% aqueous NaOH mixture (˜4:1, 400 mL). EtOAc (500 mL) was added and the mixture was allowed to warm to rt. The layers were separated keeping the solids at the interface with the aqueous layer and the aqueous layer was extracted with additional EtOAc (2×500 mL). The combined organic layers were washed with brine (300 mL), dried over Na₂SO₄, filtered, and concentrated under reduced pressure to give 4-iodo-7H-pyrrolo[2,3-d]pyrimidine as an ivory solid (15.6 g, 97% crude): LC/MS (Table 1, Method a) Rₜ=1.73 min; MS m/z: 246 (M+H)⁺.

### Step B: 4-Iodo-7-((2-(trimethylsilyl)ethoxy)methyl)-7H-pyrrolo[2,3-d]pyrimidine

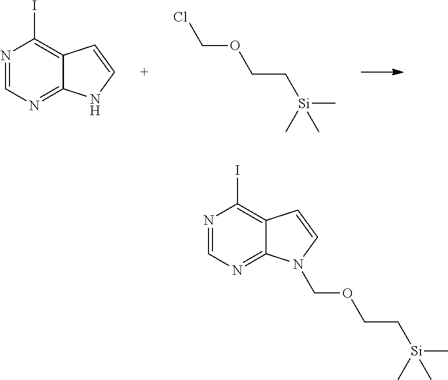

A solution of 4-iodo-7H-pyrrolo[2,3-d]pyrimidine (8.1 g, 33 1 mmol) in DMF (165 mL) was cooled to about 0° C. and NaH (60% in mineral oil) (1.98 g, 49.6 mmol) was added. After about 30 min, (2-(chloromethoxy)ethyl)trimethylsilane (7.02 mL, 39.7 mmol) was added and the reaction was continued stirring at about 0° C. After about 30 min, the reaction mixture was poured into water (500 mL) and extracted with EtOAc (2×150 mL). The combined organic layers were washed with brine (150 mL), dried over Na₂SO₄, filtered, and concentrated under reduced pressure. The crude material was purified by silica gel chromatography eluting with 0-25% EtOAc in heptane to give 4-iodo-7-((2-(trimethylsilyl)ethoxy)methyl)-7H-pyrrolo[2,3-d]pyrimidine (11.1 g, 89%, ˜92% purity) as an oil that solidified upon drying on a vacuum pump: LC/MS (Table 1, Method a) Rₜ=3.10 min; MS m/z: 376 (M+H)⁺.

### Step C: Cyclohexyl(7-((2-(trimethylsilyl)ethoxy)methyl)-7H-pyrrolo[2,3-d]pyrimidin-4-yl)methanol

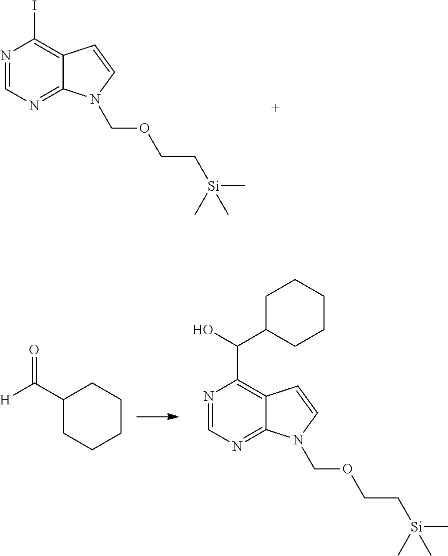

To a solution of 4-iodo-7-((2-(trimethylsilyl)ethoxy)methyl)-7H-pyrrolo[2,3-d]pyrimidine (3.0 g, 7.35 mmol) in THF (70 mL) in an ice bath at about 0° C. was added i-PrMgCl (2 M in THF, 5.52 mL, 11.03 mmol) dropwise while maintaining the internal temperature below 5° C. After about 15 min, cyclohexanecarbaldehyde (2.66 mL, 22.06 mmol) was added rapidly while maintaining the internal temperature below 10° C. After about 30 min, saturated aqueous NH₄Cl (30 mL) was added to quench the reaction. The reaction was allowed to warm to rt. The reaction was diluted with water (30 mL) and extracted with EtOAc (2×50 mL). The combined organic layers were washed with brine (100 mL), dried over Na₂SO₄, filtered, and concentrated under reduced pressure. The crude material was purified by silica gel chromatography eluting with 10-30% EtOAc in heptanes to give cyclohexyl(7-((2-(trimethylsilyl)ethoxy)methyl)-7H-pyrrolo[2,3-d]pyrimidin-4-yl)methanol (2.40 g, 58%) as a yellow oil: LC/MS (Table 1, Method a) Rₜ=2.77 min; MS m/z: 362 (M+H)⁺.

### Step D: Cyclohexyl(7-((2-(trimethylsilyl)ethoxy)methyl)-7H-pyrrolo[2,3-d]pyrimidin-4-yl)methanone

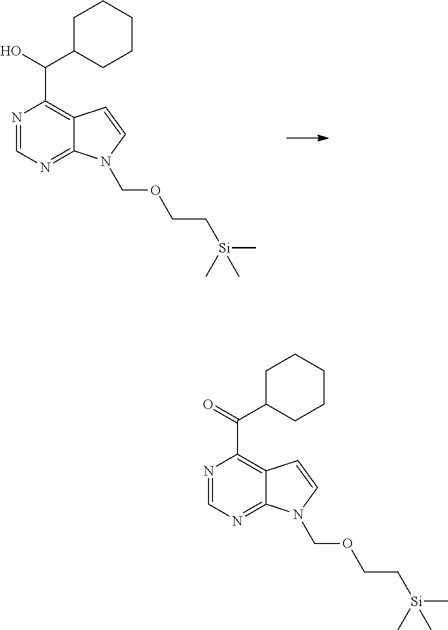

To a solution of cyclohexyl(7-((2-(trimethylsilyl)ethoxy)methyl)-7H-pyrrolo[2,3-d]pyrimidin-4-yl)methanol (2.40 g, 6.64 mmol) in DCM (35 mL) was added Dess-Martin periodinane (3.10 g, 7.30 mmol). The reaction was stirred at rt for about 30 min. The reaction was washed with aqueous saturated NaHCO₃ (2×50 mL), dried over Na₂SO₄, filtered and concentrated under reduced pressure. The crude material was purified by silica gel chromatography using 5-10% EtOAc in heptanes to give cyclohexyl(7-((2-(trimethylsilyl)ethoxy)methyl)-7H-pyrrolo[2,3-d]pyrimidin-4-yl)methanone (1.4 g, 58%) as an oil: LC/MS (Table 1, Method a) Rₜ=3.21 min; MS m/z: 360 (M+H)⁺.

### Step E: N-(Cyclohexyl(7-((2-(trimethylsilyl)ethoxy)methyl)-7H-pyrrolo[2,3-d]pyrimidin-4-yl)methyl)formamide

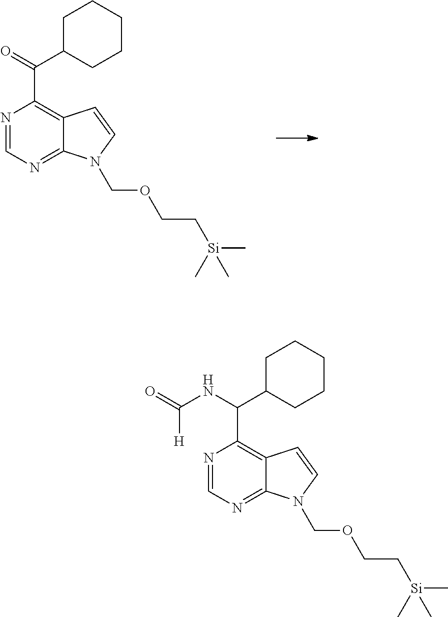

To formamide (20.0 mL, 502 mmol) at about 170° C. was added cyclohexyl(7-((2-(trimethylsilyl)ethoxy)methyl)-7H-pyrrolo[2,3-d]pyrimidin-4-yl)methanone (0.79 g, 2.2 mmol) in formic acid (2.0 mL, 52 mmol). The reaction was cooled to rt after about 30 min and then poured into water (30 mL) and made alkaline to about pH 11 with 2 N aqueous NaOH (˜15 mL). The mixture was extracted with EtOAc (3×30 mL). The combined organic layers were washed with brine (30 mL), dried over Na₂SO₄, filtered, and concentrated under reduced pressure. The crude material was purified by silica gel chromatography eluting with 20-100% DCM/MeOH/DEA (970:27:3) in DCM followed by 0-100% DCM/MeOH/DEA (950:45:5) in DCM/MeOH/DEA (970:27:3) to give N-(cyclohexyl(7-((2-(trimethylsilyl)ethoxy)methyl)-7H-pyrrolo[2,3-d]pyrimidin-4-yl)methyl)formamide with ˜6 mol % DCM and ˜4 mol % MeOH as excipients (0.75 g, 86%) as an oil: LC/MS (Table 1, Method a) Rₜ=2.75 min; MS m/z: 389 (M+H)⁺.

### Step F: 1-Cyclohexyl-7-((2-(trimethylsilyl)ethoxy)methyl)-7H-imidazo[1,5-e]pyrrolo[3,2-e]pyrimidine

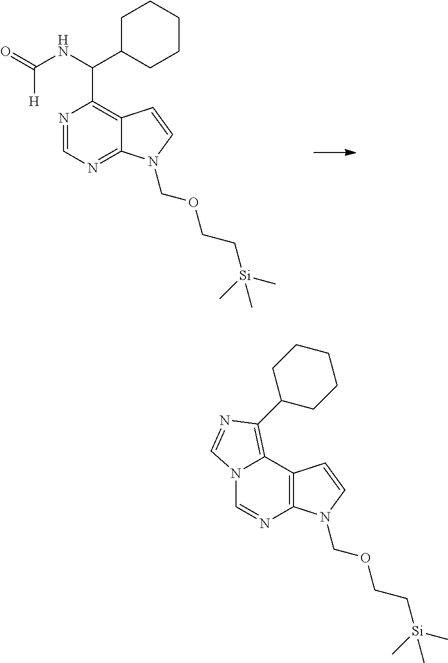

To a solution of N-(cyclohexyl(7-((2-(trimethylsilyl)ethoxy)methyl)-7H-pyrrolo[2,3-d]pyrimidin-4-yl)methyl)formamide (0.20 g, 0.52 mmol) in DCE (1.5 mL) was added a solution of POCl₃ (0.060 mL, 0.64 mmol) in DCE (1.0 mL). The reaction was heated to about 85° C. After about 1 h, the reaction was cooled to rt, diluted with DCM (20 mL), and washed with saturated aqueous NaHCO₃ (3×20 mL) and brine (20 mL). The crude material was purified by silica gel chromatography eluting with 20-80% EtOAc in heptane to give 1-cyclohexyl-7-((2-(trimethylsilyl)ethoxy)methyl)-7H-imidazo[1,5-d]pyrrolo[3,2-dpyrimidine with 15 mol % EtOAc as an excipient as an oil (0.16 g, 81%): LC/MS (Table 1, Method a) Rₜ=3.32 min; MS m/z: 371 (M+H)⁺.

### Step G: 1-Cyclohexyl-7H-imidazo[1,5-e]pyrrolo[3,2-e]pyrimidine

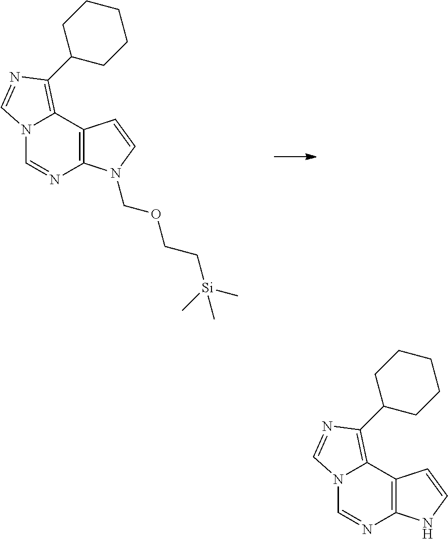

To a solution of 1-cyclohexyl-7-((2-(trimethylsilyl)ethoxy)methyl)-7H-imidazo[1,5-c]pyrrolo[3,2-e]pyrimidine (0.16 g, 0.43 mmol) in DCM (3.0 mL) was added TFA (1.0 mL, 13 mmol). After about 1.5 h at rt, the mixture was concentrated under reduced pressure. The resulting residue was dissolved in 1,4-dioxane (3.0 mL) and treated with 37% aqueous NH₄OH (3.0 mL, 28.5 mmol). The reaction mixture was heated at about 60° C. After about 30 min, the mixture was concentrated under reduced pressure. The crude material was purified by silica gel chromatography eluting with 0-100% DCM/MeOH/DEA (970:27:3) in DCM to give product with ˜50 mol % MeOH as an excipient. This solid was dissolved in a minimum amount of hot MeOH (˜2 mL) and cooled to rt while sonicating. The resulting suspension was concentrated under reduced pressure and dried in a vacuum oven at about 80° C. to give 1-cyclohexyl-7H-imidazo[1,5-c]pyrrolo[3,2-dpyrimidine (0.050 g, 48%) as a tan solid: LC/MS (Table 1, Method a) Rₜ=2.07 min; MS m/z: 241 (M+H)⁺. Jak3 IC₅₀=B; Syk IC₅₀=C.

### Example #2

### 1-Cyclohexyl-7H-pyrrolo[3,2-e][1,2,3]triazolo[1,5-c]pyrimidine

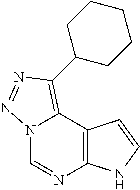

### Step A: 4-(Cyclohexyl(hydrazono)methyl)-7-((2-(trimethylsilyl)ethoxy)methyl)-7H-pyrrolo[2,3-d]pyrimidine

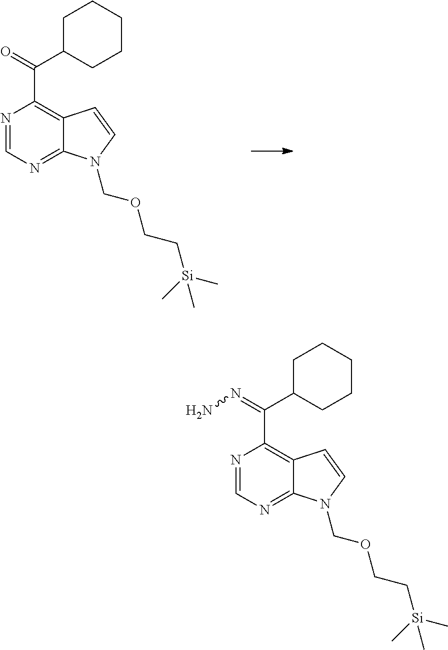

To a solution of cyclohexyl(7-((2-(trimethylsilyl)ethoxy)methyl)-7H-pyrrolo[2,3-d]pyrimidin-4-yl)methanone (0.20 g, 0.56 mmol, Example #1, Step D) in anhydrous MeOH (5 mL) was added hydrazine (1.0 mL, 32 mmol). The reaction was heated at about 65° C. for about 3 h. The reaction was partitioned between DCM (10 mL) and brine (10 mL). The organic layer was washed with brine (2×10 mL), dried over Na₂SO₄, filtered and concentrated under reduced pressure. The compound was purified on silica gel using 0-30% EtOAc in heptanes to give 4-(cyclohexyl(hydrazono)methyl)-7-((2-(trimethylsilyl)ethoxy)methyl)-7H-pyrrolo[2,3-d]pyrimidine (0.113 g, 54%) as a mixture of E/Z isomers: LC/MS (Table 1, Method a) Rₜ=2.71 and 2.80 min; MS m/z: 374 (M+H)⁺.

### Step B: 1-Cyclohexyl-7-((2-(trimethylsilyl)ethoxy)methyl)-7H-pyrrolo[3,2-e][1,2,3]triazolo[1,5-e]pyrimidine

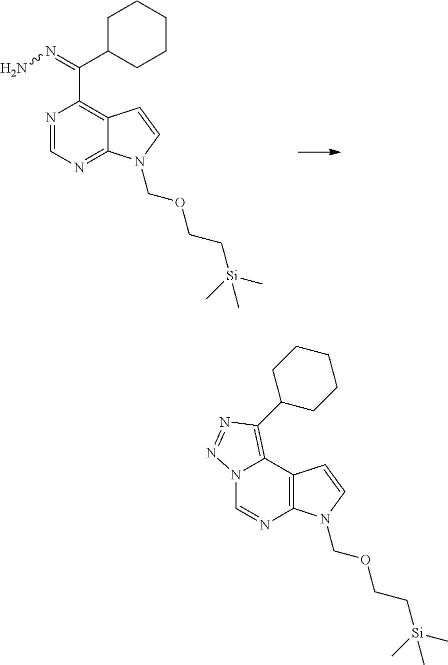

To a solution of 4-(cyclohexyl(hydrazono)methyl)-7-((2-(trimethylsilyl)ethoxy)methyl)-7H-pyrrolo[2,3-d]pyrimidine (0.31 g, 0.83 mmol) in DCM (8 mL) was added iodobenzene diacetate (0.267 g, 0.830 mmol). The reaction was stirred at rt for about 1 h. The reaction was washed with brine (10 mL), dried with Na₂SO₄, filtered, and concentrated under reduced pressure. The compound was purified on silica gel using 25-100% EtOAc in heptanes to give 1-cyclohexyl-7-((2-(trimethylsilyl)ethoxy)methyl)-7H-pyrrolo[3,2-e][1,2,3]triazolo[1,5-c]pyrimidine (0.168 g, 54%) as a white solid: LC/MS (Table 1, Method a) Rₜ=2.96 min; MS m/z: 372 (M+H)⁺.

### Step C: 1-Cyclohexyl-7H-pyrrolo[3,2-e][1,2,3]triazolo[1,5-e]pyrimidine

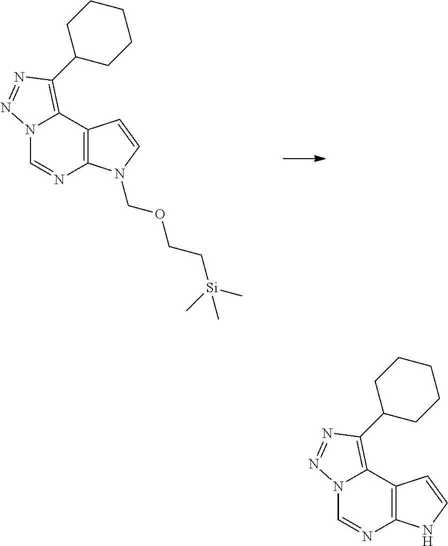

To a solution of 1-cyclohexyl-7-((2-(trimethylsilyl)ethoxy)methyl)-7H-pyrrolo[3,2-e][1,2,3]triazolo[1,5-c]pyrimidine (0.15 g, 0.40 mmol) in THF (2.0 mL) was added TBAF (1 M in THF, 1.62 mL, 1.62 mmol). The reaction was heated at about 65° C. for about 3 h. The reaction was partitioned between DCM (30 mL) and 10% aqueous AcOH (30 mL). The organic layer was washed with brine (30 mL), dried over Na₂SO₄, filtered, and concentrated under reduced pressure. The compound was purified on silica gel using 10-40% EtOAc in heptanes to give 1-cyclohexyl-7H-pyrrolo[3,2-e][1,2,3]triazolo[1,5-c]pyrimidine (0.007 g, 7%) as a tan solid: LC/MS (Table 1, Method a) Rₜ=2.02 min; MS m/z: 242 (M+H)⁺. Jak3 IC₅₀=C; Syk IC₅₀=D.

### Example #3

### Benzyl 3-(3-isopropyl-7H-imidazo[1,5-c]pyrrolo[3,2-e]pyrimidin-1-yl)piperidine-1-carboxylate

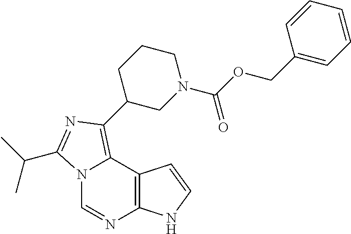

### Step A: Benzyl 3-(hydroxy(7-((2-(trimethylsilyl)ethoxy)methyl)-7H-pyrrolo[2,3-d]pyrimidin-4-yl)methyl)piperidine-1-carboxylate

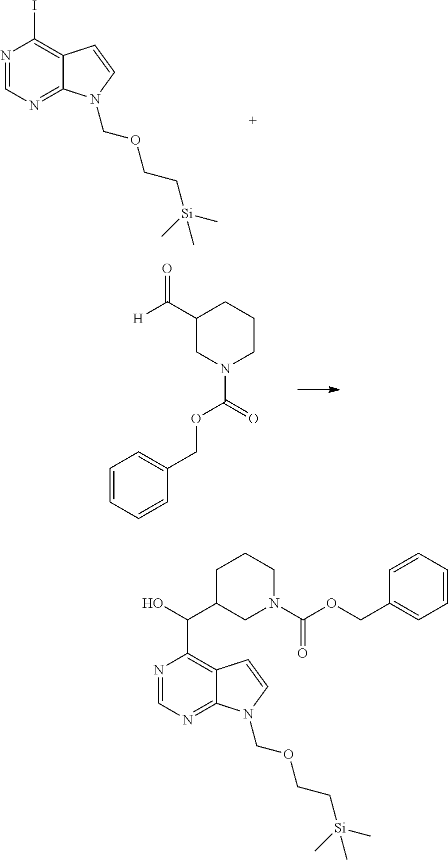

To a solution of 4-iodo-7-((2-(trimethylsilyl)ethoxy)methyl)-7H-pyrrolo[2,3-d]pyrimidine (6.00 g, 14.1 mmol, Example #1, Step B) in THF (125 mL) in an ice bath at about 0° C. was added i-PrMgCl (2 M in THF, 8.40 mL, 16 8 mmol) dropwise while maintaining the internal temperature below 5° C. After about 10 min, benzyl 3-formylpiperidine-1-carboxylate (5.10 g, 20.6 mmol, Syntech) in THF (15 mL) was added rapidly while maintaining the internal temperature below 10° C. After about 15 min, added saturated aqueous NH₄Cl (50 mL) to quench the reaction which was then warmed to rt, diluted with water (50 mL), and extracted with EtOAc (3×50 mL). The combined organic layers were washed with brine (50 mL), dried over Na₂SO₄, filtered, and concentrated under reduced pressure. The crude product was purified via silica gel chromatography eluting with 0-70% EtOAc in heptane to give benzyl 3-(hydroxy(7-((2-(trimethylsilyl)ethoxy)methyl)-7H-pyrrolo[2,3-d]pyrimidin-4-yl)methyl)piperidine-1-carboxylate (mixture of diastereomers) with 30 mol% EtOAc as an excipient as a pale yellow oil: LC/MS (Table 1, Method a) Rₜ=2.92 and 2.94 min; MS m/z: 497 (M+H)⁻.

### Step B: Benzyl 3-(7-((2-(trimethylsilyl)ethoxy)methyl)-7H-pyrrolo[2,3-d]pyrimidine-4-carbonyl)piperidine-1-carboxylate

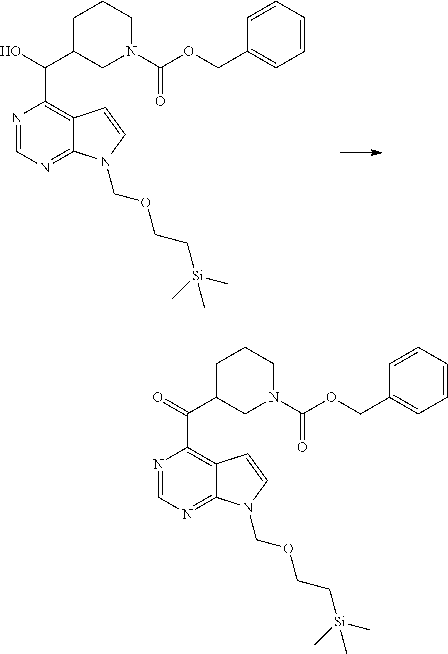

To a solution of benzyl 3-(hydroxy(7-((2-(trimethylsilyl)ethoxy)methyl)-7H-pyrrolo[2,3-d]pyrimidin-4-yl)methyl)piperidine-1-carboxylate with 30 mol % EtOAc (5.27 g, 9.67 mmol) in DCM (50 mL) was added Dess-Martin periodinane (4.92 g, 11.6 mmol). The reaction was stirred at rt. After about 1.5 h, the reaction was diluted with DCM (50 mL), washed with saturated aqueous NaHCO₃ (2×75 mL) and brine (75 mL), dried over MgSO₄, filtered, and concentrated under reduced pressure. The resulting yellow cloudy oil was purified by silica gel chromatography eluting with 0-50% EtOAc in heptane to give benzyl 3-(7-((2-(trimethylsilyl)ethoxy)methyl)-7H-pyrrolo[2,3-d]pyrimidine-4-carbonyl)piperidine-1-carboxylate with ˜17 mol % DCM as an excipient (4.20 g, 85%) as a yellow oil: LC/MS (Table 1, Method a) Rₜ=3.44 min; MS m/z: 495 (M+H)⁺.

### Step C: Benzyl 3-(3-isopropyl-7-((2-(trimethylsilyl)ethoxy)methyl)-7H-imidazo[1,5-c]pyrrolo[3,2-e]pyrimidin-1-yl)piperidine-1-carboxylate

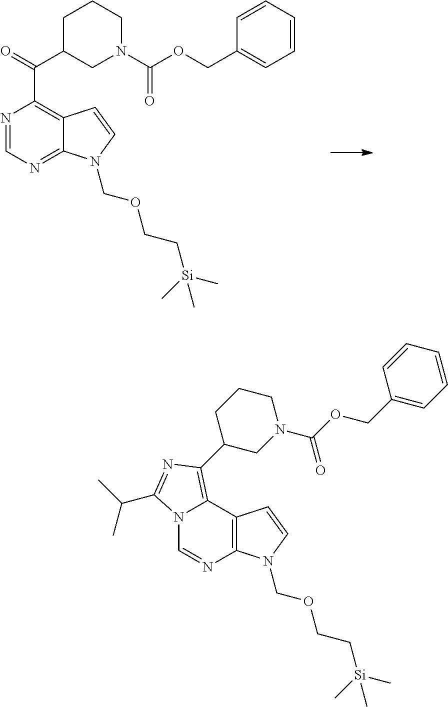

A mixture of benzyl 3-(7-((2-(trimethylsilyl)ethoxy)methyl)-7H-pyrrolo[2,3-d]pyrimidine-4-carbonyl)piperidine-1-carboxylate (0.10 g, 0.20 mmol), isobutyraldehyde (0.037 mL, 0.40 mmol), NH₄OAc (0.078 g, 1.0 mmol) and AcOH (2.0 mL) was stirred at about 110° C. After about 4 h, additional isobutyraldehyde (0.050 mL, 0.55 mmol) was added and the reaction continued heating at 110° C. After about 1.5 h, additional NH₄OAc (0.078 g, 1 0 mmol) and isobutyraldehyde (0.184 mL, 2.02 mmol) were added and the reaction continued heating at about 110° C. After about 20 h total, the reaction was cooled to rt, poured over ice-water (10 mL), warmed to rt, and extracted with DCM (3×10 mL). The combined organic layers were washed with brine, dried over MgSO₄, filtered, and concentrated under reduced pressure. The resulting brown oil was purified by silica gel chromatography eluting with 0-45% EtOAc in heptane to give benzyl 3-(3-isopropyl-7-((2-(trimethylsilyl)ethoxy)methyl)-7H-imidazo[1,5-c]pyrrolo[3,2-e]pyrimidin-1-yl)piperidine-1-carboxylate with about 50 mol% EtOAc as an excipient as a dark yellow oil (0.026 g, 22%): LC/MS (Table 1, Method a) Rₜ=3.60 min; MS m/z: 548 (M+H)⁺.

### Step D: Benzyl 3-(3-isopropyl-7H-imidazol[1,5-c]pyrrolo[3,2-e]pyrimidin-1-yl)piperidine-1-carboxylate

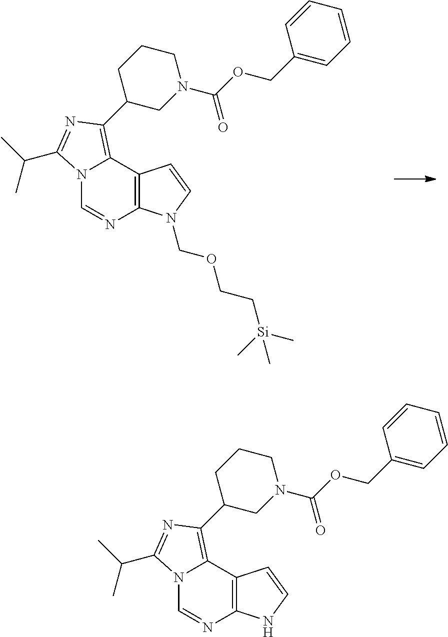

To a solution of benzyl 3-(3-isopropyl-7-((2-(trimethylsilyl)ethoxy)methyl)-7H-imidazo[1,5-c]pyrrolo[3,2-e]pyrimidin-1-yl)piperidine-1-carboxylate (0.025 g, 0.046 mmol) in DCM (0.3 mL) was added TFA (0.11 mL, 1.4 mmol). The reaction mixture was stirred at rt. After about 1.5 h, the mixture was concentrated under reduced pressure. The resulting residue was dissolved in 1,4-dioxane (0.3 mL) and treated with 37% aqueous NH₄OH (0.30 mL, 2.8 mmol). The reaction mixture was heated at about 60° C. After about 30 min, the mixture was cooled to rt and concentrated under reduced pressure. The crude product was purified via silica gel chromatography eluting with EtOAc to give product containing about 40 mol % EtOAc as a brown oil. The oil was dissolved in DCM (1 mL) and concentrated under reduced pressure twice; the resulting residue was dissolved in DCM (1 mL) and heptane (1 mL) and concentrated under reduced pressure; and then the resulting solid was triturated with heptane (2 mL), concentrated under reduced pressure, and dried in a vacuum oven to give benzyl 3-(3-isopropyl-7H-imidazo[1,5-c]pyrrolo[3,2-e]pyrimidin-1-yl)piperidine-1-carboxylate with about 15 mol % heptane as a brown solid (0.010 g, 51%): LC/MS (Table 1, Method a) Rₜ=2.40 min; MS m/z: 418 (M+H)⁺. Jak3 IC₅₀=C.

### Example #4

### Benzyl 3-(7H-imidazo[1,5-c]pyrrolo[3,2-e]pyrimidin-1-yl)piperidine-1-carboxylate

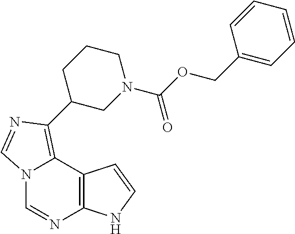

### Step A: Benzyl 3-(formamido(7-((2-(trimethylsilyl)ethoxy)methyl)-7H-pyrrolo[2,3-d]pyrimidin-4-yl)methyl)piperidine-1-carboxylate

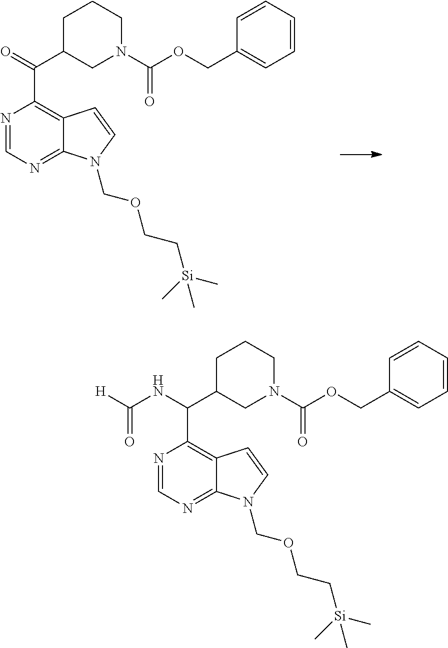

To formamide (50.0 mL, 1254 mmol) at about 170° C. was added benzyl 3-(7-((2-(trimethylsilyl)ethoxy)methyl)-7H-pyrrolo[2,3-d]pyrimidine-4-carbonyl)piperidine-1-carboxylate (3.0 g, 6.1 mmol, Example #3, Step B) in formic acid (7.0 mL, 183 mmol). The reaction was cooled to rt after about 1.5 h. Water (50 mL) was added and the mixture was made alkaline to about pH 11 with 2 N aqueous NaOH and extracted with EtOAc (3×50 mL). The combined organic layers were washed with brine (50 mL), dried over Na₂SO₄, filtered, and concentrated under reduced pressure. The crude material was purified by silica gel chromatography eluting with 20-100% DCM/MeOH/DEA (950:45:5) in DCM to give benzyl 3-(formamido(7-((2-(trimethylsilyl)ethoxy)methyl)-7H-pyrrolo[2,3-d]pyrimidin-4-yl)methyl)piperidine-1-carboxylate (˜1:1 mixture of diastereomers) as a foam (2.2 g, 69%): LC/MS (Table 1, Method a) Rₜ=2.64 and 2.71 min; MS m/z: 524 (M+H)⁺.

### Step B: Benzyl 3-(7H-imidazo[1,5-c]pyrrolo[3,2-e]pyrimidin-1-yl)piperidine-1-carboxylate

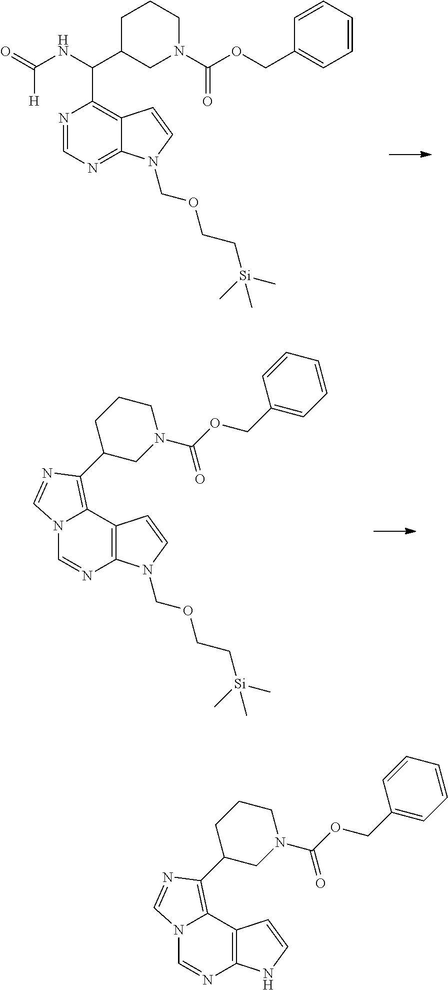

To a solution of benzyl 3-(formamido(7-((2-(trimethylsilyl)ethoxy)methyl)-7H-pyrrolo[2,3-d]pyrimidin-4-yl)methyl)piperidine-1-carboxylate (2.19 g, 4.18 mmol) in DCE (30 mL) was added POCl₃ (0.47 mL, 5.0 mmol). The reaction was heated at about 80° C. for about 2 h. The reaction was cooled to rt and diluted with DCM (5 mL). The organic layer was washed with saturated aqueous NaHCO₃ (10 mL) and brine (10 mL). The organic layer was dried over MgSO₄, filtered, and concentrated under reduced pressure. The residue was dissolved in DCM (5 mL) and TFA (1.61 mL, 20.9 mmol) was added. The reaction was stirred at rt for about 2 h. The reaction was concentrated under reduced pressure. To the residue was added 1,4-dioxane (10 mL) followed by NH₄OH (˜37%, 10 mL, 95 mmol). The reaction was heated at about 60° C. for about 30 min. The reaction was partitioned between DCM (100 mL) and brine (100 mL). The organic layer was dried over MgSO₄, filtered and concentrated under reduced pressure. The compound was purified on silica gel using 100% EtOAc to give benzyl 3-(7H-imidazo[1,5-c]pyrrolo[3,2-e]pyrimidin-1-yl)piperidine-1-carboxylate (0.91 g, 57%) as a yellow solid: LC/MS (Table 1, Method a) Rₜ=1.92 min; MS m/z: 376 (M+H)⁺. Jak3 IC₅₀=C.

### Examples #5 and #6

### (S)-1-(3-(7H-Pyrrolo[3,2-e][1,2,3]triazolo[1,5-c]pyrimidin-1-yl)piperidine-1-carbonyl)cyclopropanecarbonitrile and (R)-1-(3-(7H-pyrrolo[3,2-e][1,2,3]triazolo[1,5-c]pyrimidin-1-yl)piperidine-1-carbonyl)cyclopropanecarbonitrile

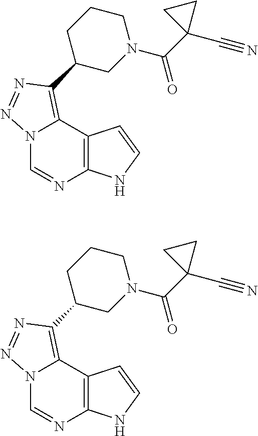

### Step A: (1-Benzylpiperidin-3-yl)(7-((2-(trimethylsilyl)ethoxy)methyl)-7H-pyrrolo[2,3-d]pyrimidin-4-yl)methanol

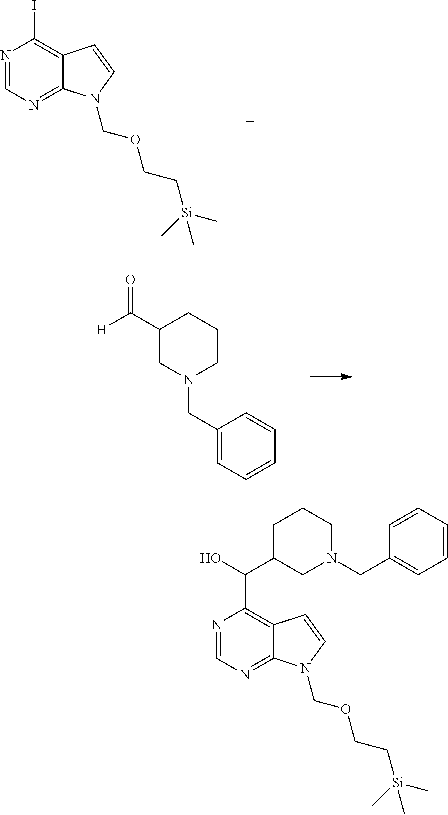

To a solution of 4-iodo-7-((2-(trimethylsilyl)ethoxy)methyl)-7H-pyrrolo[2,3-d]pyrimidine (5.34 g, 13.1 mmol, Example #1, Step B) in THF (100 mL) in an ice bath at about 0° C. was added i-PrMgCl (2 M in THF, 9.82 mL, 19 6 mmol) dropwise while maintaining the internal temperature below 5° C. After about 15 min, 1-benzylpiperidine-3-carbaldehyde (7.98 g, 39 3 mmol, Alfa Aesar) was added rapidly while maintaining the internal temperature below 10° C. After about 30 min, saturated aqueous NH₄Cl (100 mL) was added to quench the reaction. The reaction was allowed to warm to rt. The reaction was diluted with water (100 mL) and extracted with EtOAc (2×100 mL). The combined organic layers were washed with brine (100 mL), dried over Na₂SO₄, filtered, and concentrated under reduced pressure. The compound was purified by silica gel chromatography eluting with 25-100% EtOAc in heptanes to give (1-benzylpiperidin-3-yl)(7-((2-(trimethylsilyl)ethoxy)methyl)-7H-pyrrolo[2,3-d]pyrimidin-4-yl)methanol (3.51 g, 59%) as a yellow wax: LC/MS (Table 1, Method a) Rₜ=2.04 min; MS m/z: 453 (M+H)⁺.

### Step B: (1-Benzylpiperidin-3-yl)(7-((2-(trimethylsilyl)ethoxy)methyl)-7H-pyrrolo[2,3-d]pyrimidin-4-yl)methanone

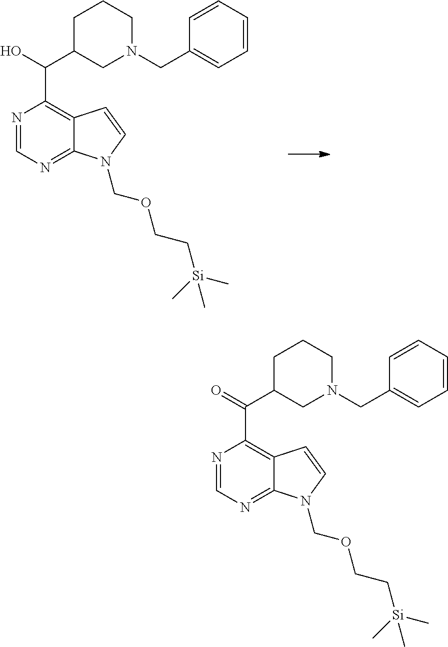

To a solution of (1-benzylpiperidin-3-yl)(7-((2-(trimethylsilyl)ethoxy)methyl)-7H-pyrrolo[2,3-d]pyrimidin-4-yl)methanol (3.50 g, 7.73 mmol) in DCM (50 mL) was added Dess-Martin periodinane (3.61 g, 8.51 mmol). The reaction was stirred at rt for about 1 h. The reaction was washed with saturated aqueous NaHCO₃ (2×100 mL), dried over Na₂SO₄, filtered, and concentrated under reduced pressure. The compound was purified on silica gel using 25-100% EtOAc in heptanes to give (1-benzylpiperidin-3-yl)(7-((2-(trimethylsilyl)ethoxy)methyl)-7H-pyrrolo[2,3-d]pyrimidin-4-yl)methanone (2.49 g, 61%) as an orange waxy solid: LC/MS (Table 1, Method a) Rₜ=2.52 min; MS m/z: 451 (M+H)⁺.

### Step C: 1-(1-Benzylpiperidin-3-yl)-7-((2-(trimethylsilyl)ethoxy)methyl)-7H-pyrrolo[3,2-e][1,2,3]triazolo[1,5-e]pyrimidine

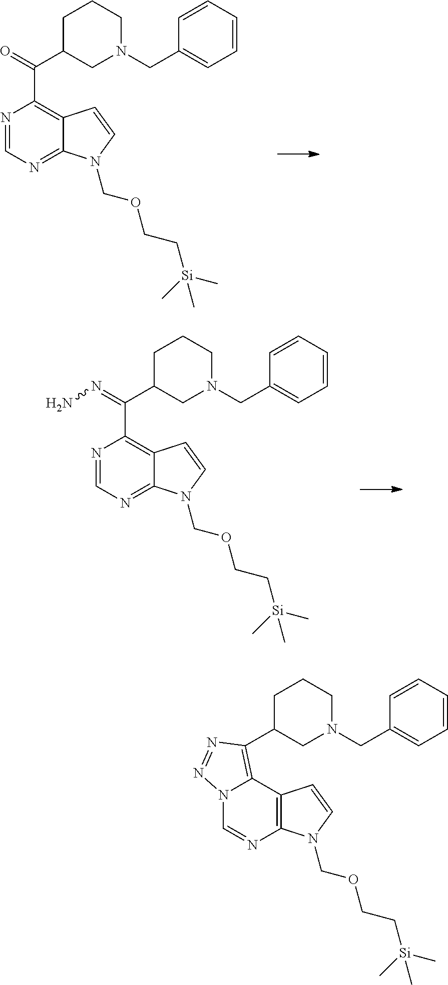

To a solution of (1-benzylpiperidin-3 -yl)(74(2-(trimethylsilyl)ethoxy)methyl)-7H-pyrrolo[2,3-d]pyrimidin-4-yl)methanone (2.49 g, 5.53 mmol) in dry MeOH (50 mL) was added hydrazine (6.94 mL, 221 mmol). The reaction was heated at about 65° C. for about 3 h. The reaction was partitioned between DCM (100 mL) and brine (50 mL). The organic layer was washed with brine (2×50 mL), dried over Na₂SO₄, filtered, and concentrated under reduced pressure. To the resulting residue was added DCM (50.0 mL) followed by iodobenzene diacetate (1.78 g, 5.53 mmol). The reaction was stirred at rt for about 16 h. The reaction was washed with brine (50 mL), dried over Na₂SO₄, filtered, and concentrated under reduced pressure. The compound was purified on silica gel using 25-100% EtOAc in heptanes to give 1-(1-benzylpiperidin-3-yl)-7-((2-(trimethylsilyl)ethoxy)methyl)-7H-pyrrolo[3,2-e][1,2,3]triazolo[1,5-c]pyrimidine (0.85 g, 33%): LC/MS (Table 1, Method a) Rₜ=2.32 min; MS m/z: 463 (M+H)⁺.

### Step D: 1-(1-Benzylpiperidin-3-yl)-7H-pyrrolo[3,2-e][1,2,3]triazolo[1,5-e]pyrimidine

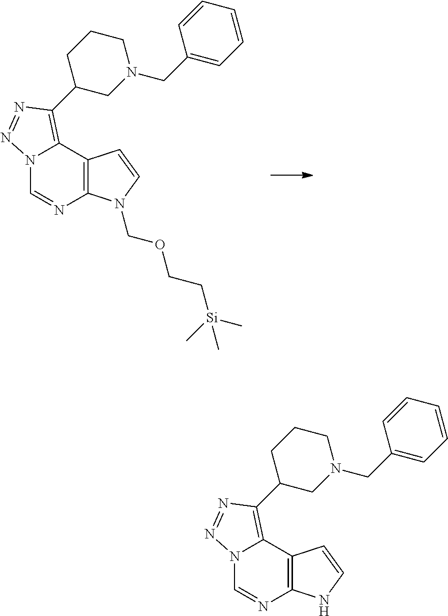

To a solution of 1-(1-benzylpiperidin-3-yl)-7-((2-(trimethylsilyl)ethoxy)methyl)-7H-pyrrolo[3,2-e][1,2,3]triazolo[1,5-c]pyrimidine (0.85 g, 1 8 mmol) in DCM (10 mL) at about 0° C. was added boron trifluoride etherate (2.33 mL, 18.4 mmol). The reaction was allowed to warm to rt. After about 2 h, the reaction was diluted with DCM (25 mL) and washed with 2 M aqueous Na₂SO₄ (25 mL). The resulting slurry of white solid was filtered through Celite® and the layers were separated. The organic layer was concentrated under reduced pressure. To the resulting residue was added 1,4-dioxane (20 mL) followed by 2 M aqueous Na₂CO₃ (20 mL, 40 mmol). The reaction was stirred at rt for about 72 h. The reaction was partitioned between EtOAc (50 mL) and water (50 mL). The organic layer was washed with brine (50 mL), dried over Na₂SO₄, filtered and concentrated under reduced pressure. The product was purified on silica gel using 50-100% EtOAc in heptanes to give 1-(1-benzylpiperidin-3-yl)-7H-pyrrolo[3,2-e][1,2,3]triazolo[1,5-c]pyrimidine (0.14 g, 23%) as a white solid: LC/MS (Table 1, Method a) Rₜ=1.32 min; MS m/z: 333 (M+H)⁺.

### Step E: 1-(3-(7H-Pyrrolo[3,2-e][1,2,3]triazolo[1,5-c]pyrimidin-1-yl)piperidine-1-carbonyl)cyclopropanecarbonitrile

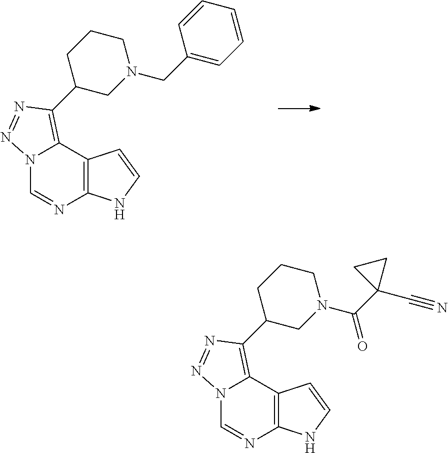

To a solution of 1-(1-benzylpiperidin-3-yl)-7H-pyrrolo[3,2-e][1,2,3]triazolo[1,5-c]pyrimidine (0.140 g, 0.421 mmol) in MeOH (20 mL) was added Pearlman's catalyst (0.059 g, 0.084 mmol). The reaction was stirred at about 60° C. under an atmosphere of hydrogen via balloon for about 1 h. The reaction was allowed to cool to rt. The reaction was filtered through Celite and concentrated under reduced pressure. The resulting residue was dissolved in DCM (5 mL) and 1-cyanocyclopropanecarboxylic acid (0.07 g, 0.62 mmol), DIEA (0.108 mL, 0.619 mmol) and EDC (0.119 g, 0.619 mmol) were added respectively. The reaction was stirred at rt for about 16 h. The reaction was diluted with DCM (5 mL) and washed with saturated aqueous NaHCO₃ (10 mL). The organic layer was dried over Na₂SO₄, filtered and concentrated under reduced pressure. The compound was purified on silica gel using 20-100% EtOAc in heptanes to give 1-(3-(7H-pyrrolo[3,2-e][1,2,3]triazolo[1,5-c]pyrimidin-1-yl)piperidine-1-carbonyt)cyclopropanecarbonitrile (0.061 g, 43%) as a white solid: LC/MS (Table 1, Method a) Rₜ=1.57 min; MS m/z: 336 (M+H)⁺. Jak3 IC₅₀=C.

### Step F: (S)-1-(3-(7H-Pyrrolo[3,2-e][1,2,3]triazolo[1,5-c]pyrimidin-1-yl)piperidine-1-carbonyl)cyclopropanecarbonitrile and (R)-1-(3-(7H-pyrrolo[3,2-e][1,2,3]triazolo[1,5-c]pyrimidin-1-yl)piperidine-1-carbonyl)cyclopropanecarbonitrile

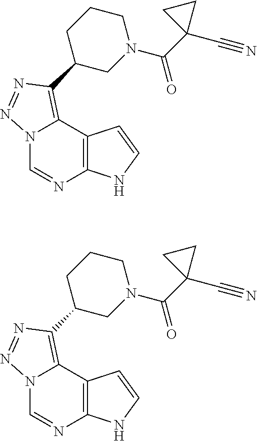

1-(3-(7H-Pyrrolo[3,2-e][1,2,3]triazolo[1,5-c]pyrimidin-1-yl)piperidine-1-carbonyl)cyclopropane-carbonitrile (0.60 g, 0.18 mmol) was separated using chiral HPLC to give (S)-1-(3-(7H-pyrrolo[3,2-e][1,2,3]triazolo[1,5-c]pyrimidin-1-yl)piperidine-1-carbonyt)cyclopropanecarbonitrile (Table 1, Method g, Rₜ=9.9 min, or =negative) (0.010 g, 7%) and (R)-1-(3-(7H-pyrrolo[3,2-e][1,2,3]triazolo[1,5-c]pyrimidin-1-yl)piperidine-1-carbonyt)cyclopropane-carbonitrite (Table 1, Method g, Rₜ=14.5 min, or =positive) (0.008 g, 6%). The preceding stereochemistry was arbitrarily assigned. Jak3 IC₅₀=C for enantiomer with or =negative and Jak3 IC₅₀=B for enantiomer with or =positive.

### Example #7

### 4-(7H-Imidazo[1,2-c]pyrrolo[3,2-e]pyrimidin-5-ylamino)-N-propylbenzamide

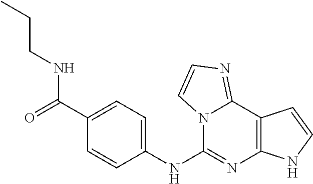

### Step A: 4-Amino-N-propylbenzamide

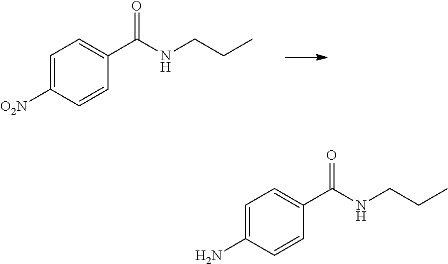

4-Nitro-N-propylbenzamide (3.31 g, 15 9 mmol) was dissolved in EtOAc (125 mL) and EtOH (125 mL) and passed through the H-Cube® at 1 mL/min equipped with a 10% Pd/C catcart (Thales Nano) at full hydrogen and temperature set to about 50° C. The solvent was stripped off and the solid was dried overnight in a vacuum oven at about 50° C. to provide 4-amino-N-propylbenzamide (2.58 g, 91%): ¹H NMR (DMSO-d₆) δ 7.93 (t, J=5.48 Hz, 1H); 7.54 (d, J=8.57 Hz, 2H); 6.51 (d, J=8.67 Hz, 2H) 5.54 (br s, 2H) 3.13 (m, 2H) 1.47 (m, 2H) 0.85 (t, J=8.49 Hz, 3H).

### Step B: 2,4-Dichloro-7-((2-(trimethylsilyl)ethoxy)methyl)-7H-pyrrolo[2,3-d]pyrimidine

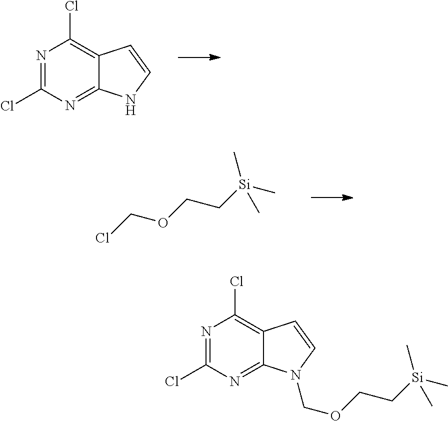

To a suspension of NaH (60 wt % in mineral oil, 0.64 g, 16 mmol) in DMF (50 mL) at 0° C. was added a solution of 2,4-dichloro-7H-pyrrolo[2,3-d]pyrimidine (2.5 g, 13 mmol, prepared as described in WO 2009/026107, Example #1) in DMF (25 mL) followed by the addition of (2-(chloromethoxy)ethyl)trimethylsilane (3.07 mL, 17.3 mmol). The reaction mixture was stirred at about 0° C. for about 1 h and then warmed to rt. Water (about 75 mL) was added and the mixture was extracted with EtOAc (about 75 mL). The organic layer was washed with brine (about 75 mL), dried over MgSO₄ and filtered. The filtrate was passed through a silica plug eluting with EtOAc/hexanes (1:1) to provide 2,4-dichloro-7-((2-(trimethylsdyl)ethoxy)methyl)-7H-pyrrolo[2,3-d]pyrimidine (4.2 g, 99%): ¹H NMR (DMSO-d₆) δ 7.89 (d, J=3.69 Hz, 1H), 6.76 (d, J=3.69 Hz, 1H), 5.59 (s, 2H), 3.54-3.50 (m, 2H), 0.85-0.81 (m, 2H), -0.10 (s, 9H).

### Step C: 2-Chloro-7-((2-(trimethylsilyl)ethoxy)methyl)-7H-pyrrolo[2,3-d]pyrimidin-4-amine

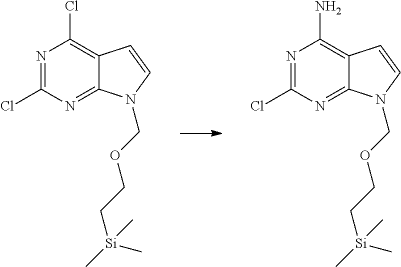

A Uniqsis Flowsyn fitted with a T-mixer, a 14 mL loop, and a backpressure 100 psi regulator was primed with 1,4-dioxane. To line A of the flow reactor was added a 0.5 M solution of 2,4-dichloro-7-((2-(trimethylsilyl)ethoxy)methyl)-7H-pyrrolo[2,3-d]pyrimidine (0.17 g, 0.50 mmol) in 1,4-dioxane. To line B was added a solution of ammonium hydroxide. The system was run at a flow rate 1 mL/min with a resonance time about 14 min at about 120° C. The reaction mixture was collected and concentrated in vacuo to provide 2-chloro-7-((2-(trimethylsilyl)-ethoxy)methyl)-7H-pyrrolo[2,3-d]pyrimidin-4-amine (0.15 g, 99% crude): LC/MS (Table 1, Method b) Rₜ=1.75 min; MS m/z 299 (M+H)⁺.

### Step D: 5-Chloro-7-((2-(trimethylsilyl)ethoxy)methyl)-7H-imidazo[1,2-e]pyrrolo[3,2-e]pyrimidine

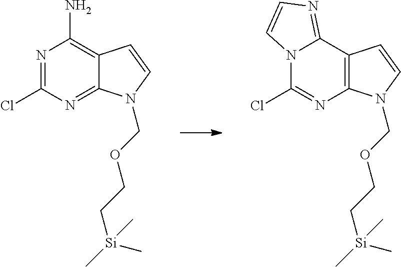

A vial was charged with 2-chloro-7-((2-(trimethylsilyl)ethoxy)methyl)-7H-pyrrolo[2,3-d]pyrimidin-4-amine (0.14 g, 0.46 mmol) dissolved in 1,4-dioxane (0.5 mL). 2-Chloroacetaldehyde (1 mL, 0.46 mmol) was added to the solution and heated in a Biotage single-mode microwave for about 5 min at about 120° C. The reaction was concentrated in vacuo and the crude product was added to a silica gel column and was eluted with 0-5% MeOH in DCM to provide 5-chloro-7-((2-(trimethylsilyl)ethoxy)methyl)-7H-imidazo[1,2-c]pyrrolo[3,2-e]pyrimidine (0.12 g, 82%): (Table 1, Method b) Rₜ=1.55 min; MS m/z 323 (M+H)⁺.

### Step E: N-Propyl-4-(7-((2-(trimethylsilyl)ethoxy)methyl)-7H-imidazo[1,2-e]pyrrolo[3,2-e]pyrimidin-5-ylamino)benzamide

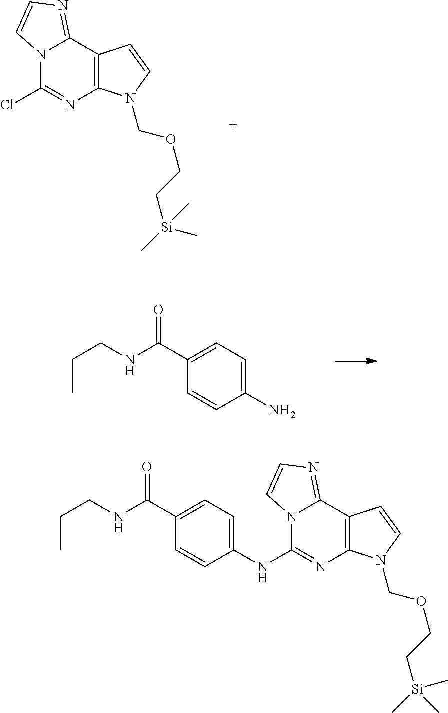

5-Chloro-7-((2-(trimethylsilyl)ethoxy)methyl)-7H-imidazo[1,2-c]pyrrolo[3,2-e]pyrimidine (0.12 g, 0.37 mmol), 4-amino-N-propylbenzamide (0.066 g, 0.37 mmol), X-Phos (0.011 g, 0.022 mmol), Pd₂dba₃ (0.018 g, 0.022 mmol) and K₂CO₃ (0.061 g, 0.44 mmol) was added to a 10 mL vial. t-BuOH (2 mL) was then added and the tube was sealed. The tube was evacuated and purged with nitrogen (3×), and stirred at about 80° C. for about 16 h. The mixture was filtered, the filter pad was washed with EtOAc, and then the solvent was removed in vacuo. The crude material was added to a silica gel column and was eluted with 40-60% EtOAc in hexane to provide N-propyl-4-(7-((2-(trimethylsityl)ethoxy)methyl)-7H-imidazo[1,2-c]pyrrolo[3,2-e]pyrimidin-5-ylamino)benzamide after drying in a vacuum oven overnight (0.084 g, 49%): (Table 1, Method b) Rₜ=1.69 min; MS m/z 465 (M+H)⁺.

### Step F: 4-(7H-Imidazo[1,2-e]pyrrolo[3,2-e]pyrimidin-5-ylamino)-N-propylbenzamide

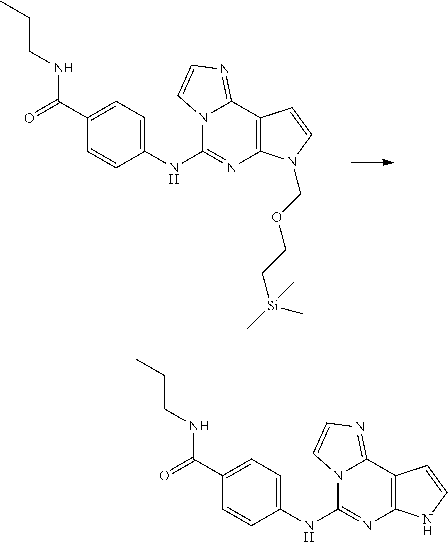

N-propyl-4-(7-((2-(trimethylsilyl)ethoxy)methyl)-7H-imidazo[1,2-c]pyrrolo[3,2-e]pyrimidin-5-ylamino)benzamide (0.064 g, 0.14 mmol) in DCM (2 mL) was treated with TFA (0.50 mL, 6.5 mmol) and the mixture was stirred for about 16 h at rt. The reaction was concentrated in vacuo, NH₄OH (0.11 mL, 1.1 mmol) in 1,4-dioxane (1 mL) was added, and the mixture was heated to about 60° C. while stirring. After about 1 h, the reaction was diluted with water (25 mL). The reaction mixture was concentrated under a warm stream of nitrogen. The crude material was loaded on a TLC plate (1.0 mm) The plate was developed using 10% MeOH in DCM to provide 4-(7H-imidazo[1,2-c]pyrrolo[3,2-e]pyrimidin-5-ylamino)-N-propylbenzamide (0.0046 g, 10%): LC/MS (Table 1, Method b) Rₜ=1.02 min; MS m/z 335 (M+H)⁺. Syk IC₅₀=D.

### Example #8

### 3-(4-Methoxyphenyl)-7H-pyrrolo[3,2-e][1,2,4]triazolo[4,3-e]pyrimidine

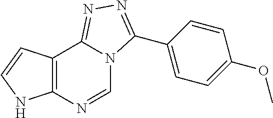

### Step A: 4-Hydrazinyl-7H-pyrrolo[2,3-d]pyrimidine hydrochloride

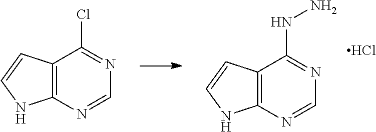

4-Chloro-7H-pyrrolo[2,3-d]pyrimidine (0.250 g, 1.63 mmol, ArkPharm) in 1,4-dioxane (4 mL) with hydrazine hydrate (0.326 g, 6.51 mmol) was heated in a microwave at about 120° C. for about 1 h. The mixture was cooled and diluted with Et₂O (2 mL). The solids were collected by filtration then washed with Et₂O (2 mL). The material was dried to a constant weight under vacuum at about 60° C. to give 4-hydrazinyl-7H-pyrrolo[2,3-d]pyrimidine hydrochloride (0.23 g, 76%): LC/MS (Table 1, Method d) Rₜ=0.53 min; MS m/z 150 (M+H)⁺.

### Step B: 3-(4-Methoxyphenyl)-7H-pyrrolo[3,2-e][1,2,4]triazolo[4,3-c]pyrimidine

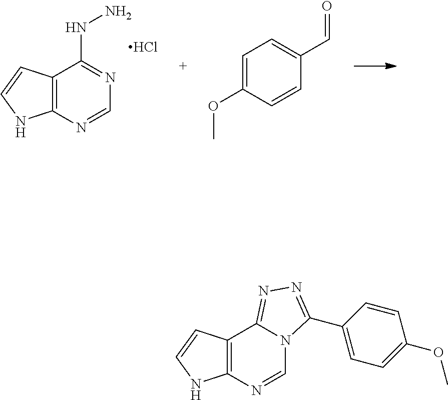

4-Hydrazinyl-7H-pyrrolo[2,3-d]pyrimidine hydrochloride (0.115 g, 0.620 mmol) in DCM (4 mL) and MeOH (1 mL) was treated with DIEA (0.162 mL, 0.929 mmol) and 4-methoxybenzaldehyde (0.093 g, 0.68 mmol). The mixture was stirred for about 2 h then iodobenzene diacetate (0.220 g, 0.682 mmol) was added. After about 1 h, the material was purified directly by preparative reverse phase HPLC (Table 1, Method 1). The fractions containing the title compound were collected then concentrated under reduced pressure to remove most of the MeCN. The mixture was basified with saturated aqueous NaHCO₃ and then extracted with EtOAc (2×20 mL). The combined organic layers were concentrated under reduced pressure and then purified using a 10 g silica column eluting with 94:6 DCM/MeOH. The material was triturated with Et₂O (3 mL) and the solid collected by filtration and dried to a constant weight under vacuum at about 60° C. to give 3-(4-methoxyphenyl)-7H-pyrrolo[3,2-e][1,2,4]triazolo[4,3-c]pyrimidine (0.005 g, 3%): LC/MS (Table 1, Method a) Rₜ=1.56 min; MS m/z 266 (M+H)⁺. Syk IC₅₀=D.

### Example #9

### 3-(4-(Methylsulfonyl)phenyl)-7H-pyrrolo[3,2-e][1,2,4]triazolo[4,3-c]pyrimidine

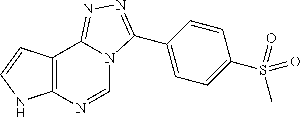

### Step A: 4-Chloro-7-((2-(trimethylsilyl)ethoxy)methyl)-7H-pyrrolo[2,3-d]pyrimidine

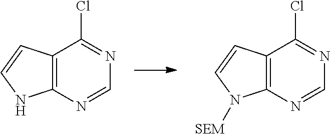

4-Chloro-7H-pyrrolo[2,3-d]pyrimidine (2.0 g, 13 mmol) in DMF (25 mL) was treated with NaH (60 wt% in mineral oil, 0.573 g, 14.3 mmol) at rt. After about 15 min, SEMCl (2.43 mL, 13.7 mmol) was added and the mixture stirred for about 30 min. The mixture was concentrated under reduced pressure and then the material was partitioned between EtOAc (30 mL) and water (30 mL). The organic layer was separated and the aqueous layer was extracted with EtOAc (25 mL). The combined organic layers were dried over MgSO₄, filtered, and concentrated under reduced pressure to give 4-chloro-7-((2-(trimethylsilyl)ethoxy)methyl)-7H-pyrrolo[2,3-d]pyrimidine (4.33 g, 117% crude): LC/MS (Table 1, Method a) Rₜ=2.93 min; MS m/z 284 (M+H)⁺.

### Step B: 4-Hydrazinyl-7-((2-(trimethylsilyl)ethoxy)methyl)-7H-pyrrolo[2,3-d]pyrimidine

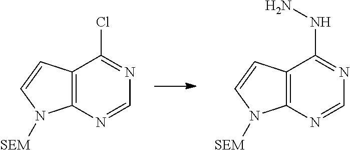

4-Chloro-7-((2-(trimethylsilyl)ethoxy)methyl)-7H-pyrrolo[2,3-d]pyrimidine (4.30 g, 15.2 mmol) was dissolved in 1,4-dioxane (50 mL) then treated with hydrazine hydrate (1.52 g, 30.3 mmol). The mixture was heated in an oil bath at about 100° C. for about 3 h. The mixture was cooled and concentrated under reduced pressure. The material was suspended in water (40 mL) and then basified with 50% aqueous NaOH. The material was extracted twice with EtOAc (100 mL and 75 mL). The combined organic layers were dried over MgSO₄, filtered, and concentrated under reduced pressure to give 4-hydrazinyl-7-((2-(trimethylsilyl)ethoxy)-methyl)-7H-pyrrolo[2,3-d]pyrimidine (3.74 g, 88%): LC/MS (Table 1, Method a) Rₜ=1.71 min; MS m/z 280 (M+H)⁺.

### Step C: 3-(4-(Methylsulfonyl)phenyl)-7-((2-(trimethylsilyl)ethoxy)methyl)-7H-pyrrolo[3,2-e][1,2,4]triazolo[4,3-e]pyrimidine

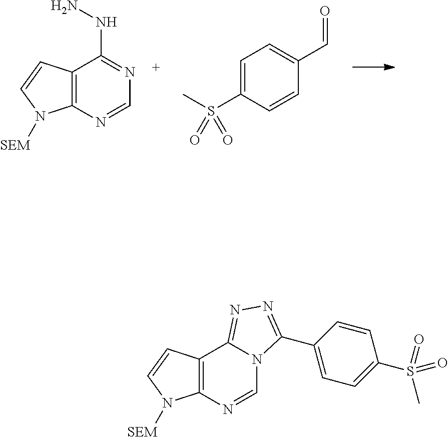

A mixture of 4-hydrazinyl-7-((2-(trimethylsilyl)ethoxy)methyl)-7H-pyrrolo[2,3-d]pyrimidine (0.100 g, 0.358 mmol) and 4-(methylsulfonyl)benzaldehyde (0.069 g, 0.376 mmol, AKSci) in DCM (5 mL) with 1 drop of AcOH was stirred at about 30° C. After about 2 h, the solution was treated with iodobenzene diacetate (0.173 g, 0.537 mmol). The mixture was stirred at rt for about 3 h, concentrated under reduced pressure, and purified using a 10 g silica column eluting with 96:4 DCM/MeOH to give 3-(4-(methylsulfonyl)phenyl)-7-((2-(trimethylsilyl)ethoxy)methyl)-7H-pyrrolo[3,2-e][1,2,4]triazolo[4,3-c]pyrimidine (0.090 g, 57%): LC/MS (Table 1, Method a) Rₜ=2.29 min; MS m/z 444 (M+H)⁺.

### Step D: 3-(4-(Methylsulfonyl)phenyl)-7H-pyrrolo[3,2-e][1,2,4]triazolo[4,3-e]pyrimidine

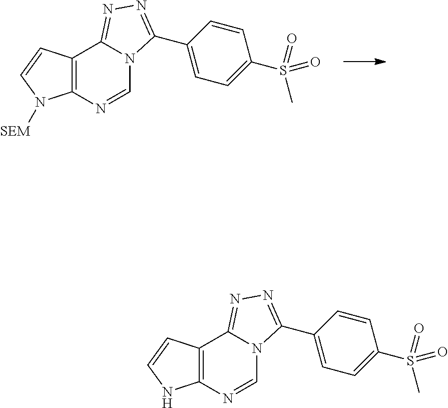

3 -(4-(Methylsulfonyl)phenyl)-7-((2-(trimethylsilyl)ethoxy)methyl)-7H-pyrrolo[3,2-e][1,2,4]triazolo[4,3-c]pyrimidine (0.090 g, 0.20 mmol) was dissolved in DCM (4 mL) and then treated with TFA (2 mL, 26 mmol). The mixture was stirred for about 3 h and then concentrated under reduced pressure. The material was dissolved in 1,4-dioxane (4 mL) and treated with concentrated aqueous NH₄OH (26 wt %, 2 mL, 13.35 mmol). The mixture was stirred at rt for about 1 h and then concentrated under reduced pressure. The material was triturated with water (5 mL) and the solids were collected by filtration and washed with water (2 mL). The material was dried to a constant weight under vacuum at about 60° C. to give 3-(4-(methylsulfonyl)phenyl)-7H-pyrrolo[3,2-e][1,2,4]triazolo[4,3-c]pyrimidine (0.048 g, 76%): LC/MS (Table 1, Method a) Rₜ=1.48 min; MS m/z 314 (M+H)⁺. Syk IC₅₀=D.

### Example #10

### 2-(4-(7H-Imidazo[1,2-e]pyrrolo[3,2-e]pyrimidin-3-yl)phenyl)propan-2-ol

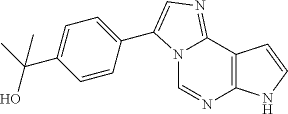

### Step A: 7-((2-(Trimethylsilyl)ethoxy)methyl)-7H-pyrrolo[2,3-d]pyrimidin-4-amine

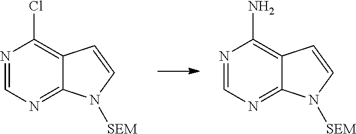

In a steel pressure vessel was added 4-chloro-7-((2-(trimethylsilyl)ethoxy)methyl)-7H-pyrrolo[2,3-d]pyrimidine (3.15 g, 11.1 mmol, Example #9, Step A) and 40% aqueous NH₄OH (21.6 mL, 222 mmol, JTBaker) in 1,4-dioxane (30 mL) and the mixture was heated under pressure to about 100° C. for about 17 h. The mixture was cooled to rt and concentrated in vacuo. The residue was triturated with Et₂O (15 mL) and filtered. Additional solids formed in the Et₂O and were also collected by filtration. The solids were combined to give 7-((2-(trimethylsilyl)ethoxy)methyl)-7H-pyrrolo[2,3-d]pyrimidin-4-amine (1.67 g, 57%): LC/MS (Table 1, Method e) Rₜ=0.65 min; MS m/z 265 (M+H)⁺.

### Step B: 7-((2-(Trimethylsilyl)ethoxy)methyl)-7H-imidazo[1,2-e]pyrrolo[3,2-e]pyrimidine

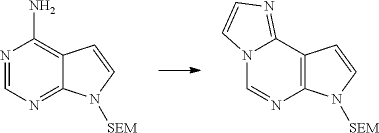

To a flask was added 7-((2-(trimethylsilyl)ethoxy)methyl)-7H-pyrrolo[2,3-d]pyrimidin-4-amine (1.45 g, 5.48 mmol) and 2-chloroacetaldehyde (2.12 mL, 16 5 mmol) in EtOH (25 mL) and mixture was heated to about 80° C. for about 1 h. The mixture was concentrated in vacuo then purified by 40 g silica column, eluting with 100% EtOAc followed by 5% MeOH in DCM, to give 7-((2-(trimethylsilyl)ethoxy)methyl)-7H-imidazo[1,2-c]pyrrolo[3,2-e]pyrimidine (1.13 g, 71%): LC/MS (Table 1, Method e) Rₜ=0.69 min; MS m/z 289 (M+H)⁺.

### Step C: 3-Bromo-7-((2-(trimethylsilyl)ethoxy)methyl)-7H-imidazo[1,2-e]pyrrolo[3,2-e]pyrimidine

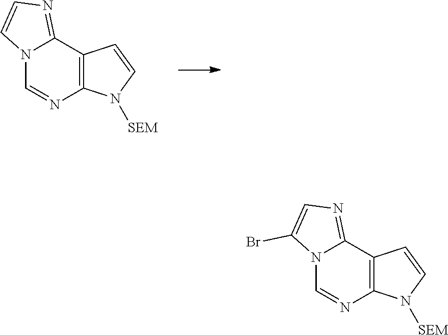

A mixture of 7-((2-(trimethylsilyl)ethoxy)methyl)-7H-imidazo[1,2-c]pyrrolo[3,2-e]pyrimidine (1.02 g, 3.54 mmol) and DMF (22 mL) was cooled to about 0° C. NBS (0.566 g, 3.18 mmol) was added and the reaction was stirred for about 10 min. The resulting precipitate was collected and washed with water and then dried to give 3-bromo-7-((2-(trimethylsilyl)ethoxy)methyl)-7H-imidazo[1,2-c]pyrrolo[3,2-e]pyrimidine (0.942 g, 73%): LC/MS (Table 1, Method e) R_{f}=0.79 min; MS m/z 367, 369 (M+H)⁺.

### Step D: 3-Bromo-7H-imidazo[1,2-e]pyrrolo[3,2-e]pyrimidine

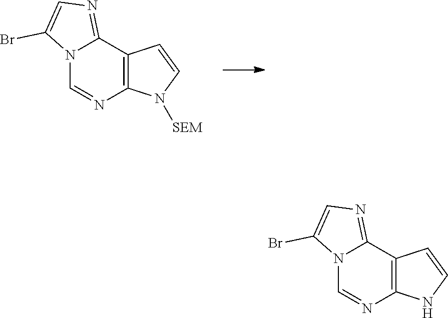

To a flask was added 3-bromo-7-((2-(trimethylsilyl)ethoxy)methyl)-7H-imidazo[1,2-c]pyrrolo[3,2-e]pyrimidine (0.40 g, 1.09 mmol) and TFA (1.26 mL, 16.33 mmol) in DCM (5 mL) and the mixture was stirred at rt for about 17 h. The mixture was concentrated in vacuo and then 1,4-dioxane (2 mL) and concentrated NH₄OH (1.41 mL, 10.9 mmol, JTBaker) were added. The mixture was heated to about 60° C. for about 30 min. The mixture was cooled to rt and the solid was filtered and rinsed with water. Additional solids also precipitated from the filtrate and were collected by filtration. The solids were dried to constant weight and combined to provide 3-bromo-7H-imidazo[1,2-c]pyrrolo[3,2-dpyrimidine (0.125 g, 48%): LC/MS (Table 1, Method a) Rₜ=1.31 min; MS m/z 237, 239 (M+H)⁺. Syk IC₅₀=D.

### Step E: 2-(4-(4,4,5,5-Tetramethyl-1,3,2-dioxaborolan-2-yl)phenyl)propan-2-ol

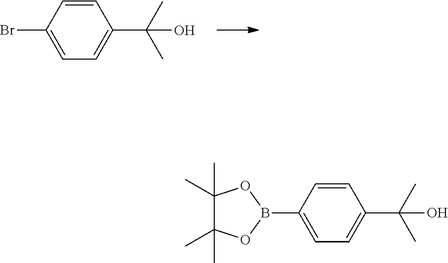

A flask was charged with 2-(4-bromophenyl)propan-2-ol (5.00 g, 23 3 mmol, prepared according to Bioorg. Med. Chem. Lett. 2007, 17, 662), bis(pinacolato)diboron (6.49 g, 25 6 mmol), KOAc (6.84 g, 69.7 mmol), PdCl₂(dppf)-CH₂Cl₂ (0.949 g, 1.16 mmol) and DMSO (155 mL). The mixture was heated to about 80° C. for about 4 h. After cooling to rt, the mixture was partitioned between brine (400 mL) and EtOAc (100 mL). The organic layer was isolated and the aqueous phase was extracted with two further portions of EtOAc (2×50 mL). The organic layers were combined, washed with brine (5×100 mL), dried over anhydrous MgSO₄ and concentrated in vacuo. The crude material was purified by silica gel flash chromatography with a gradient of 0 to 100% EtOAc in hexanes to give 2-(4-(4,4,5,5-tetramethyl-1,3,2-dioxaborolan-2-yl)phenyl)propan-2-ol (2.76 g, 45%): ¹H NMR (d-DMSO) δ 7.80 (s, J=8.0 Hz, 2H), 7.50 (s, J=8.0 Hz, 2H), 1.58 (s, 6H), 1.34 (s, 12H).

### Step F: 2-(4-(7H-Imidazo[1,2-c]pyrrolo[3,2-e]pyrimidin-3-yl)phenyl)propan-2-ol

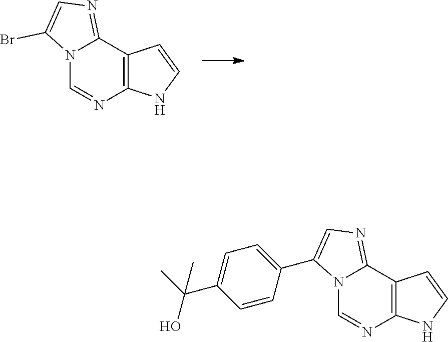

To a microwave vial was added 3-bromo-7H-imidazo[1,2-c]pyrrolo[3,2-e]pyrimidine (0.092 g, 0.39 mmol, Step D), 2-(4-(4,4,5,5-tetramethyl-1,3,2-dioxaborolan-2-yl)phenyl)propan-2-ol (0.203 g, 0.776 mmol, Step E) and Cs₂CO₃ (0.379 g, 1.16 mmol) in 1,4-dioxane (2 mL), water (0.5 mL) and EtOH (1 mL). SiliaCat DPP-Pd® (0.031 mmol, Silicycle) was then added and mixture was heated to about 150° C. for about 30 min in a microwave, filtered and rinsed with MeOH. The resulting filtrate was concentrated and purified directly by HPLC (Table 1, Method h). The material was concentrated, lyophilized and then purified a second time by HPLC (Table 1, Method i), concentrated and lyophilized to provide 2-(4-(7H-imidazo[1,2-c]pyrrolo[3,2-e]pyrimidin-3-yl)phenyl)propan-2-ol (0.007 g, 6%): LC/MS (Table 1, Method a) Rₜ=1.32 min; MS m/z 293 (M+H)⁺. Syk IC₅₀=B.

### Example #11

### 2-(Pyridin-3-yl)-7H-imidazo[1,2-c]pyrrolo[3,2-e]pyrimidine

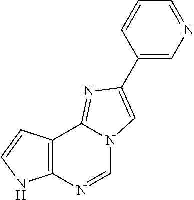

### Step A: 2-(Pyridin-3-yl)-7-((2-(trimethylsilyl)ethoxy)methyl)-7H-imidazo[1,2-e]pyrrolo[3,2-e]pyrimidine

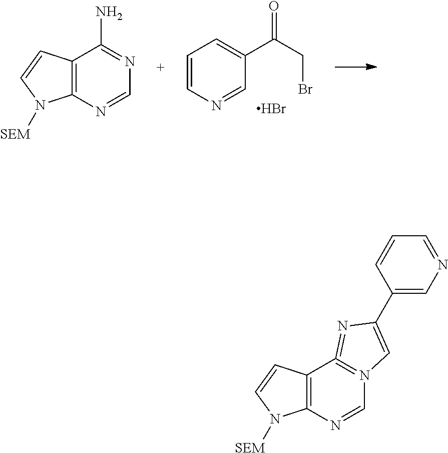

7-((2-(Trimethylsilyl)ethoxy)methyl)-7H-pyrrolo[2,3-d]pyrimidin-4-amine (0.500 g, 1.89 mmol, Example #10, Step A) and 2-bromo-1-(pyridin-3-yl)ethanone hydrobromide (0.531 g, 1.89 mmol) in MeCN (10 mL) were treated with TEA (0.659 mL, 4.73 mmol) then heated to about 80° C. for about 3 h. 2-Bromo-1-(pyridin-3-yl)ethanone hydrobromide (0.531 g, 1.89 mmol) was added and The mixture was heated at about 80° C. for about 14 h. TEA (0.264 mL, 1.89 mmol) was added then the mixture was heated at about 80° C. for about 3 h. The mixture was diluted with water (50 mL) and saturated aqueous NaHCO₃ (15 mL). The mixture was extracted with EtOAc (2×50 mL), the combined organic layers were dried over anhydrous MgSO₄ and then filtered. The filtrate was concentrated under reduced pressure. The material was purified on a 10 g silica column eluting with 9:1 DCM/MeOH to give 2-(pyridin-3-yl)-7-((2-(trimethylsilyl)ethoxy)methyl)-7H-imidazo[1,2-c]pyrrolo[3,2-e]pyrimidine (0.080 g, 12%): LC/MS (Table 1, Method d) Rₜ=2.49 min; MS m/z 366 (M+H)⁺.

### Step B: 2-(Pyridin-3-yl)-7H-imidazo[1,2-e]pyrrolo[3,2-e]pyrimidine

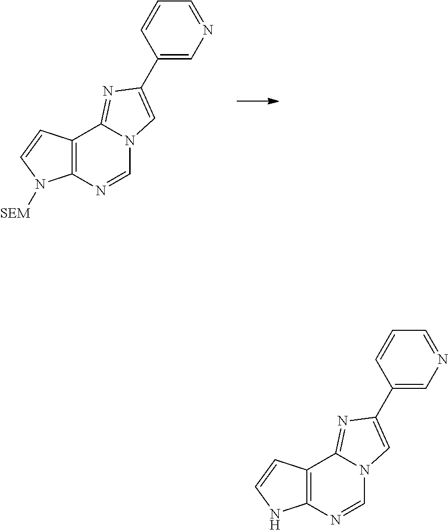

2-(Pyridin-3-yl)-7-((2-(trimethylsilyl)ethoxy)methyl)-7H-imidazo[1,2-c]pyrrolo[3,2-e]pyrimidine (0.080 g, 0.22 mmol) in DCM (3 mL) was treated with TFA (2.5 mL, 32 mmol) then stirred for about 18 h at rt. The solvents were removed under reduced pressure and then the material was dissolved in 1,4-dioxane (3 mL) and treated with 26% aqueous NH₄OH (2 mL, 14 mmol). The mixture was stirred for about 18 h at rt and then concentrated under reduced pressure. The material was purified by preparative reverse phase HPLC (Table 1, Method c). The fractions were collected, combined and then concentrated under reduced pressure to remove most/all of the MeCN. The mixture was basified with saturated aqueous NaHCO₃ and the solids were collected by filtration and dried to a constant weight at about 70° C. under vacuum to give 2-(pyridin-3-yl)-7H-imidazo[1,2-c]pyrrolo[3,2-e]pyrimidine (0.023 g, 45%): LC/MS (Table 1, Method a) Rₜ=1.32 min; MS m/z 236 (M+H)⁺. Syk IC₅₀=C.

### Example #12

### 8-(Pyridin-4-yl)-7H-imidazo[1,2-c]pyrrolo[3,2-e]pyrimidine

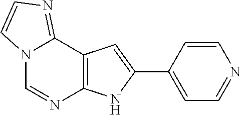

### Step A: 4-Chloro-6-iodo-7-((2-(trimethylsilyl)ethoxy)methyl)-7H-pyrrolo[2,3-d]pyrimidine

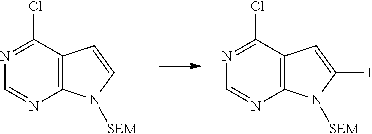

To a solution of 4-chloro-7-((2-(trimethylsilyl)ethoxy)methyl)-7H-pyrrolo[2,3-d]pyrimidine (5.56 g, 19.6 mmol, Example #9, Step A) in THF (20 mL) was added LDA (2 M in THF/heptane/ethylbenzene, 19.6 mL, 39.2 mmol) dropwise at about −78° C. The reaction was stirred at about −78° C. for about 1 h and then a solution of I₂ (9.94 g, 39 2 mmol) in THF (10 mL) was added dropwise. After about 30 min, the dry ice bath was removed and the reaction was warmed to rt. The reaction was quenched by the addition of saturated aqueous Na₂S₂O₃ and extracted with EtOAc. The combined organic layers were washed with water and brine, dried over anhydrous MgSO₄, filtered, and concentrated under reduced pressure. The crude material was purified via silica gel chromatography eluting with 0-20% EtOAc in hexane to give 4-chloro-6-iodo-7-((2-(trimethylsdyl)ethoxy)methyl)-7H-pyrrolo[2,3-d]pyrimidine (2.65 g, 33%) as a white solid: ¹H NMR (300 MHz, d-DMSO) δ 8.62 (s, 1H), 7.12 (s, 1H), 5.61 (s, 2H), 3.61-3.45 (m, 2H), 0.90-0.74 (m, 2H), −0.11 (s, 9H).

### Step B: 6-Iodo-7-((2-(trimethylsilyl)ethoxy)methyl)-7H-pyrrolo[2,3-d]pyrimidin-4-amine

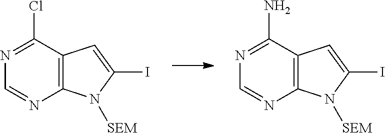

To a 250 mL stainless steel pressure bottle containing ammonium hydroxide (20 mL, 514 mmol) were added 4-chloro-6-iodo-7-((2-(trimethylsilyl)ethoxy)methyl)-7H-pyrrolo[2,3-d]pyrimidine (2.55 g, 6.22 mmol) and 1,4-dioxane (20 mL). The reaction mixture was stirred for about 16 h at about 60° C. followed by heating at about 85° C. for about 16 h. The mixture was then filtered through a nylon membrane and concentrated to dryness. The resulting white solid was triturated with water. The solid was collected by filtration, rinsed with water, and dried to give 6-iodo-7-((2-(trimethylsdyl)ethoxy)methyl)-7H-pyrrolo[2,3-d]pyrimidin-4-amine (2.25 g, 93%) as a white solid: ¹H NMR (300 MHz, d-DMSO) δ 8.01 (s, 1H), 7.09 (s, 2H), 6.93 (s, 1H), 5.44 (s, 2H), 3.57-3.41 (m, 2H), 0.88-0.72 (m, 2H), −0.10 (s, 9H).

### Step C: 6-(Pyridin-4-yl)-7-((2-(trimethylsilyl)ethoxy)methyl)-7H-pyrrolo[2,3-d]pyrimidin-4-amine

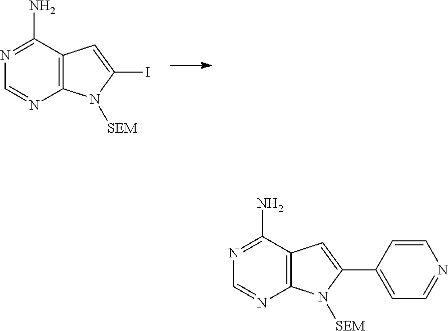

A mixture of 6-iodo-7-((2-(trimethylsilyl)ethoxy)methyl)-7H-pyrrolo[2,3-d]pyrimidin-4-amine (0.21 g, 0.54 mmol), pyridin-4-ylboronic acid (0.092 g, 0.75 mmol), PdCl₂(dppf)•DCM (0.041 g, 0.050 mmol), and sodium carbonate (0.16 g, 1 5 mmol) in DMF (2 mL) and water (1 mL) was heated at about 80° C. for about 4 h. The reaction was cooled to rt, diluted with water, and extracted with EtOAc. The organic phase was washed with brine, dried over anhydrous MgSO₄, and concentrated to dryness. The residue was purified by flash column chromatography eluting with 0-3% MeOH in DCM to give 6-(pyridin-4-yl)-7-((2-(trimethylsdyl)ethoxy)methyl)-7H-pyrrolo[2,3-d]pyrimidin-4-amine (0.12 g, 66%) as a tan solid: ¹H NMR (300 MHz, DMSO) δ 8.65 (dd, J=4.5, 1.6 Hz, 2H), 8.15 (s, 1H), 7.70 (dd, J=4.6, 1.6 Hz, 2H), 7.25 (bs, 2H), 7.01 (s, 1H), 5.58 (s, 2H), 3.60 (t, J=8.0 Hz, 2H), 0.84 (t, J=8.0 Hz, 2H), −0.12 (s, 9H).

### Step D: 6-(Pyridin-4-yl)-7H-pyrrolo[2,3-d]pyrimidin-4-amine

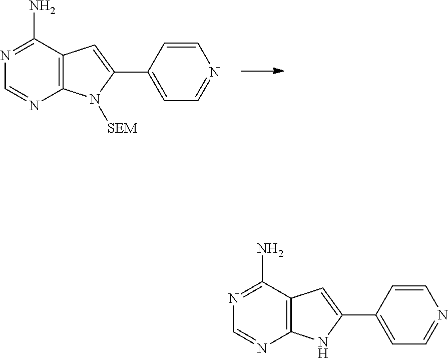

A mixture of 6-(pyridin-4-yl)-7-((2-(trimethylsilyl)ethoxy)methyl)-7H-pyrrolo[2,3-d]pyrimidin-4-amine (0.11 g, 0.33 mmol), EtOH (0.5 mL), and concentrated HCl (0.5 mL) was heated at about 80° C. overnight. The reaction mixture was then concentrated to dryness. To the resulting residue was added saturated aqueous NaHCO₃ and extracted with i-PrOH/CHCl₃ (1:3). The organic layer was washed with brine and concentrated under reduced pressure to give 6-(pyridin-4-yl)-7H-pyrrolo[2,3-d]pyrimidin-4-amine (0.060 g, 87%) as a yellow solid: ¹H NMR (300 MHz, DMSO) δ 12.25 (s, 1H), 8.59 (dd, J=4.7, 1.5 Hz, 2H), 8.09 (s, 1H), 7.71 (dd, J=4.7, 1.5 Hz, 2H), 7.20 (d, J=1.7 Hz, 1H), 7.15 (s, 2H).

### Step E: 8-(Pyridin-4-yl)-7H-imidazo[1,2-e]pyrrolo[3,2-e]pyrimidine

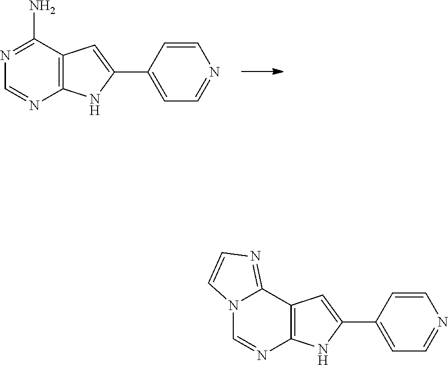

A mixture of 6-(pyridin-4-yl)-7H-pyrrolo[2,3-d]pyrimidin-4-amine (0.055 g, 0.26 mmol) and 2-chloroacetaldehyde (40% in water, 0.50 mL, 0.26 mmol) was stirred at about 30° C. overnight. To the reaction was added ethanol and then the mixture was concentrated to dryness. The resulting residue was triturated with ether and filtered to give 8-(pyridin-4-yl)-7H-imidazo[1,2-c]pyrrolo[3,2-e]pyrimidine (0.055 g, 90%) as a yellow solid: ¹H NMR (300 MHz, DMSO) δ 13.96 (s, 1H), 9.44 (s, 1H), 8.88 (d, J=6.1 Hz, 2H), 8.36 (d, J=6.1 Hz, 2H), 8.33 (d, J=1.8 Hz, 1H), 8.02 (d, J=1.5 Hz, 1H), 7.89 (s, 1H); LC/MS (Table 1, Method d) Rₜ=1.13 min; MS m/z 236 (M+H)⁺. Syk IC₅₀=C.

### Example #13

### 1-(3-(3-(4-Isopropylphenyl)-7H-imidazo[1,5-c]pyrrolo[3,2-e]pyrimidin-1-yl)piperidin-1-yl)ethanone

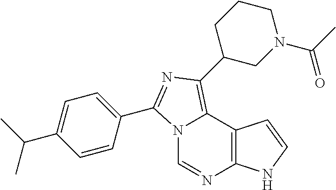

### Step A: Benzyl 3-(7-tosyl-7H-imidazo[1,5-c]pyrrolo[3,2-e]pyrimidin-1-yl)piperidine-1-carboxylate

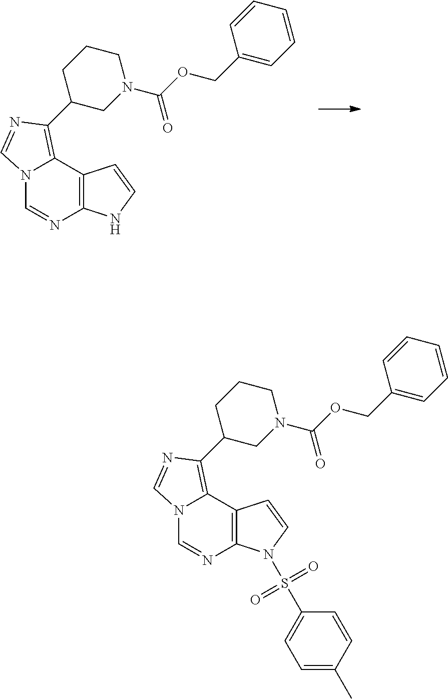

A solution of benzyl 3-(7H-imidazo[1,5-c]pyrrolo[3 ,2-e]pyrimidin-1-yl)piperidine-1-carboxylate (0.91 g, 2.4 mmol) (Example #4) in THF (15 mL) was cooled to about 0° C. in an ice bath. NaH (60% in mineral oil) (0.102 g, 2.55 mmol) was added. After about 30 min, a solution of Ts-Cl (0.508 g, 2.67 mmol) in THF (5 mL) was added. The reaction mixture was stirred at rt for 2 h. The reaction was partitioned with EtOAc (50 mL) and brine (50 mL), the organic layer was dried with Na₂SO₄, filtered and concentrated. The residue was recrystallized from EtOAc (20 mL)/ether (50 mL) to afford benzyl 3-(7-tosyl-7H-imidazo[1,5-c]pyrrolo[3,2-e]pyrimidin-1-yl)piperidine-1-carboxylate (1.00 g, 76%). LC/MS (Table 1, Method a) Rₜ=2.47 min; MS m/z: 530 (M+H)⁺.

### Step B: Benzyl 3-(3-bromo-7-tosyl-7H-imidazo[1,5-e]pyrrolo[3,2-e]pyrimidin-1-yl)piperidine-1-carboxylate

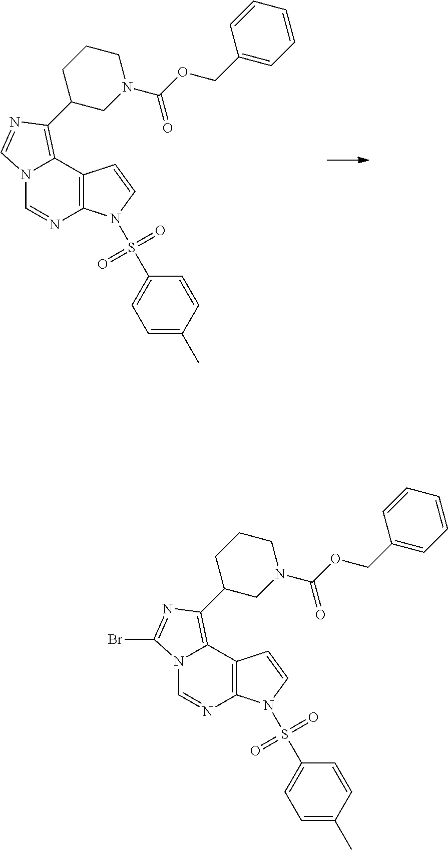

At 0° C., a solution of NBS (0.299 g, 1.68 mmol) in THF (9 mL) was added to a solution of benzyl 3-(7-tosyl-7H-imidazo[1,5-c]pyrrolo[3,2-e]pyrimidin-1-yl)piperidine-1-carboxylate (0.890 g, 1.68 mmol) in THF (36 mL). After 30 min, the reaction was partitioned between EtOAc (80 mL) and saturated aqueous NaHCO₃ (2×50 mL), the organic layer was dried with Na₂SO₄, filtered and concentrated. The residue was purified on silica gel (40 g) eluting with 10-40%

EtOAc in heptanes to give benzyl 3-(3-bromo-7-tosyl-7H-imidazo[1,5-c]pyrrolo[3,2-e]pyrimidin-1-yl)piperidine-1-carboxylate (0.90 g, 87%) as a pale yellow solid: LC/MS (Table 1, Method a) Rₜ=2.74 min; MS m/z 609 (M+H)⁺.

### Step C: Benzyl 3-(3-(4-(2-hydroxypropan-2-yl)phenyl)-7-tosyl-7H-imidazo[1,5-c]pyrrolo[3,2-e]pyrimidin-1-yl)piperidine-1-carboxylate

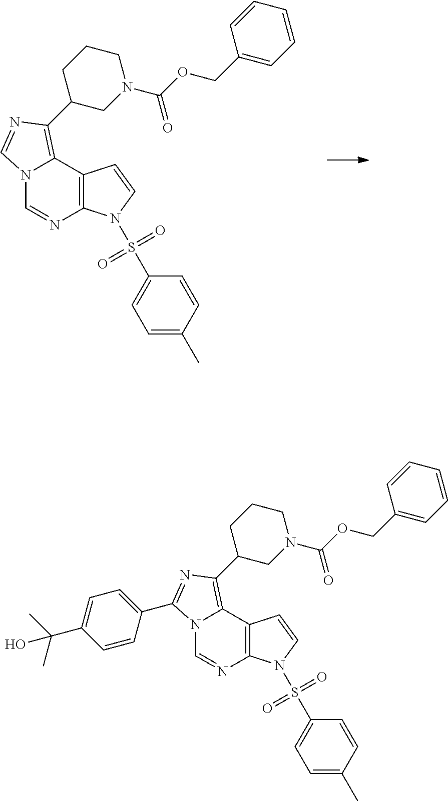

Benzyl 3-(3-bromo-7-tosyl-7H-imidazo[1,5-c]pyrrolo[3,2-e]pyrimidin-1-yl)piperidine-1-carboxylate (0.10 g, 0.16 mmol) and 2-(4-(4,4,5,5-tetramethyl-1,3,2-dioxaborolan-2-yl)phenyl)propan-2-ol (0.043 g, 0.16 mmol) were combined in 1,4-dioxane (1.6 mL) to give a yellow suspension. A solution of cesium carbonate (0.134 g, 0.411 mmol) in water (0.4 mL) was added. Nitrogen was bubbled through the mixture. Bis(triphenylphosphine)palladium(II) dichloride (0.008 g, 0.012 mmol) was added, the mixture further flushed with nitrogen, then the mixture was heated at about 75° C. for about 18 h. The mixture was concentrated, the residue was purified by silica gel chromatography eluting with 20-70% EtOAc/DCM to provide benzyl 3-(3-(4-(2-hydroxypropan-2-yl)phenyl)-7-tosyl-7H-imidazo[1,5-c]pyrrolo[3,2-e]pyrimidin-1-yl)piperidine-1-carboxylate (0.072 g, 66%); LC/MS (Table 1, Method a) Rₜ=2.80 min; MS m/z 664 (M+H)⁺.

### Step D: 3-(4-Isopropylphenyl)-1-(piperidin-3-yl)-7-tosyl-7H-imidazo[1,5-e]pyrrolo[3,2-e]pyrimidine

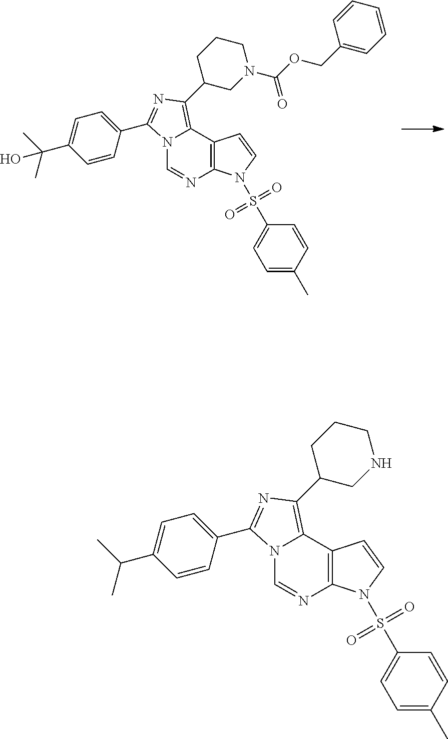

To a solution of benzyl 3-(3-(4-(2-hydroxypropan-2-yl)phenyl)-7-tosyl-7H-imidazo[1,5-c]pyrrolo[3,2-e]pyrimidin-1-yl)piperidine-1-carboxylate (0.065 g, 0.098 mmol) in acetonitrile (0.98 mL) was added iodotrimethylsilane (0.133 mL, 0.979 mmol). The mixture was heated at about 75° C. for about 18 h. The reaction was cooled to ambient temperature and the crude mixture was purified directly by preparative reverse phase HPLC (Table 1, Method h) to provide 3-(4-isopropylphenyl)-1-(piperidin-3-yl)-7-tosyl-7H-imidazo[1,5-c]pyrrolo[3,2-e]pyrimidine (0.04 g, 80%); LC/MS (Table 1, Method a) Rₜ=2.29 min; MS m/z 514 (M+H)⁺.

### Step E: 1-(3-(3-(4-Isopropylphenyl)-7-tosyl-7H-imidazo[1,5-e]pyrrolo[3,2-e]pyrimidin-1-yl)piperidin-1-yl)ethanone

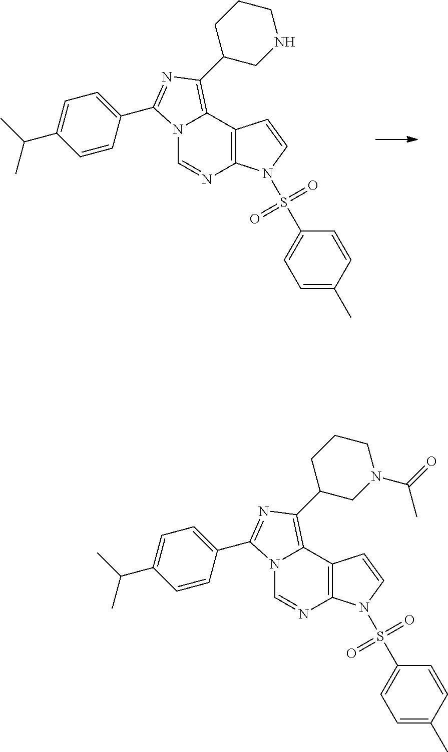

3-(4-Isopropylphenyl)-1-(piperidin-3-yl)-7-tosyl-7H-imidazo[1,5-c]pyrrolo[3,2-e]pyrimidine (0.040 g, 0.078 mmol) was stirred in DCM (2 mL) to give a brown suspension. TEA (0.022 mL, 0.16 mmol) was added followed by addition of acetic anhydride (0.015 mL, 0.16 mmol). The reaction mixture was stirred at rt for about 18 h. The mixture was then concentrated and the residue was purified by silica gel chromatography eluting with 0-60% EtOAc/DCM to provide 1-(3-(3-(4-isopropylphenyl)-7-tosyl-7H-imidazo[1,5-c]pyrrolo[3,2-e]pyrimidin-1-yl)piperidin-1-yl)ethanone (0.02 g, 46%) as an orange oil; LC/MS (Table 1, Method a) Rₜ=2.88 min; MS m/z 556 (M+H)⁺.

### Step F: 1-(3-(3-(4-Isopropylphenyl)-7H-imidazo[1,5-c]pyrrolo[3,2-e]pyrimidin-1-yl)piperidin-1-yl)ethanone

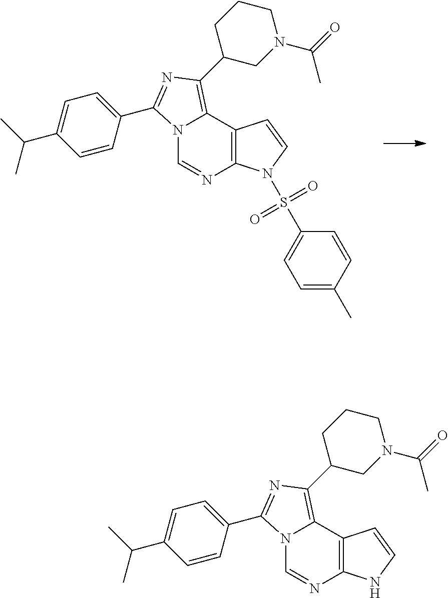

1-(3-(3-(4-Isopropylphenyl)-7-tosyl-7H-imidazo[1,5-c]pyrrolo [3,2-e]pyrimidin-1-yl)piperidin-1-yl)ethanone (0.02 g, 0.036 mmol) was stirred in THF (1.44 mL) to give an orange solution. TBAF (1.0 M in THF, 0.036 mL, 0.036 mmol) was added. The mixture was heated at about 70° C. for about 1.5 h. The reaction was cooled to ambient temperature and the mixture was deposited on silica gel (0.5 g) and purified by silica gel chromatography eluting with 0-10% MeOH/DCM to provide 1-(3-(3-(4-isopropylphenyl)-7H-imidazo[1,5-c]pyrrolo[3,2-e]pyrimidin-1-yl)piperidin-1-yl)ethanone (0.004 g, 26%) as a yellow solid; LC/MS (Table 1, Method a) Rₜ=2.15 min; MS m/z 402 (M+H)⁺. Syk IC₅₀=C.

What is claimed:

1. A compound of Formula (I) 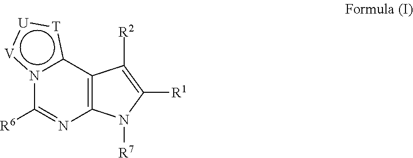
pharmaceutically acceptable salts, pro-drugs and biologically active metabolites thereof wherein
T is N or CR³;
U is N or CR⁴;
V is N or CR⁵;
R¹ is H, optionally substituted (C₁-C₆)alkyl, optionally substituted (C₃-C₆)cycloalkyl, optionally substituted heterocyclyl, optionally substituted aryl, or optionally substituted heteroaryl;
R² is H or optionally substituted (C₁-C₆)alkyl, NR^{a}R^{b}, OR^{b}, CONR^{a}R^{b}, NR^{a}COR^{b}, optionally substituted (C₃-C₆)cycloalkyl, optionally substituted heterocyclyl, optionally substituted aryl, or optionally substituted heteroaryl;
R³ is H, Br, Cl, F, optionally substituted (C₁-C₆)alkyl, optionally substituted (C₃-C₆)cycloalkyl, optionally substituted heterocyclyl, optionally substituted aryl, or optionally substituted heteroaryl;
R⁴ is H, Br, Cl, F, optionally substituted (C₁-C₆)alkyl, optionally substituted (C₃-C₆)cycloalkyl, optionally substituted heterocyclyl, optionally substituted aryl, or optionally substituted heteroaryl;
R⁵ is H, Br, Cl, F, optionally substituted (C₁-C₆)alkyl, optionally substituted (C₃-C₆)cycloalkyl, optionally substituted heterocyclyl, optionally substituted aryl, or optionally substituted heteroaryl;
R⁶ is H or NR^{a}R^{b};
R⁷ is H, optionally substituted-(CH₂)ₙ—P(═O)(OR^{a})(OR^{a}), optionally substituted —(CH₂)ₙ—O—P(═O)(OR^{a})(OR^{a}), optionally substituted-(CH₂)ₙ—P(═O)(OR^{a})(R^{a}), —CH═CH—P(═O)(OR^{a})(OR^{a});
R^{a} is H;
R^{b} is H, optionally substituted (C₁-C₆)alkyl, optionally substituted aryl, optionally substituted heteroaryl, optionally substituted (C₃-C₆)cycloalkyl, or optionally substituted heterocyclyl; and
n is 0, 1 or 2.

2. The compound of claim 1 wherein
R¹ is H or optionally substituted heteroaryl;
R³ is H, optionally substituted (C₁-C₆)alkyl, optionally substituted (C₃-C₆)cycloalkyl, or optionally substituted heterocyclyl;
R⁴ is H, optionally substituted (C₁-C₆)alkyl or optionally substituted heteroaryl; and
R⁵ is H, Br, optionally substituted (C₁-C₆)alkyl or optionally substituted aryl.

3. The compound of claim 2 wherein T is N.

4. The compound of claim 3 wherein U is CR⁴.

5. The compound of claim 4 wherein V is CR⁵.

6. The compound of claim 5 wherein R⁵ is Br or optionally substituted phenyl.

7. The compound of claim 5 wherein R⁶ is NR^{a}R^{b} wherein R^{a} is H and R^{b} is optionally substituted phenyl.

8. The compound of claim 5 wherein R¹ is optionally substituted pyridinyl.

9. The compound of claim 5 wherein R⁴ is optionally substituted phenyl or optionally substituted pyridinyl.

10. The compound of claim 3 wherein U is N.

11. The compound of claim 10 wherein V is CR⁵.

12. The compound of claim 11 wherein R⁵ is optionally substituted phenyl.

13. The compound of claim 2 wherein T is CR³.

14. The compound of claim 13 wherein U is N.

15. The compound of claim 14 wherein V is N.

16. The compound of claim 15 wherein R³ is optionally substituted cyclohexyl or optionally substituted piperidinyl.

17. The compound of claim 14 wherein V is CR⁵.

18. The compound of claim 17 wherein R³ is optionally substituted cyclohexyl or optionally substituted piperidinyl and R⁵ is H, optionally substituted (C₁-C₆)alkyl or optionally substituted phenyl.

19. The compound of claim 1 wherein the compound is
1-Cyclohexyl-7H-imidazo[1,5-c]pyrrolo[3,2-e]pyrimidine;
1-Cyclohexyl-7H-pyrrolo[3,2-e][1,2,3]triazolo[1,5-c]pyrimidine;
Benzyl 3 -(3 -isopropyl-7H-imidazo [1,5-c]pyrrolo[3,2-e]pyrimidin-1-yl)piperidine-1-carboxylate;
Benzyl 3-(7H-imidazo[1,5-c]pyrrolo[3,2-e]pyrimidin-1-yl)piperidine-1-carboxylate;
(S)-1-(3-(7H-Pyrrolo[3,2-e][1,2,3 ]triazolo[1,5-c]pyrimidin-1-yl)piperidine-1-carbonyl)cyclopropanecarbonitrile;
(R)-1-(3-(7H-pyrrolo[3,2-e][1,2,3]triazolo[1,5-c]pyrimidin-1-yl)piperidine-1-carbonyl)cyclopropanecarbonitrile;
4-(7H-Imidazo[1,2-c]pyrrolo[3,2-e]pyrimidin-5-ylamino)-N-propylbenzamide;
3-(4-Methoxyphenyl)-7H-pyrrolo[3,2-e][1,2,4]triazolo[4,3-c]pyrimidine;
3-(4-(Methylsulfonyl)phenyl)-7H-pyrrolo[3,2-e][1,2,4]triazolo[4,3-c]pyrimidine;
2-(4-(7H-Imidazo[1,2-c]pyrrolo[3,2-e]pyrimidin-3-yl)phenyl)propan-2-ol;
2-(Pyridin-3-yl)-7H-imidazo[1,2-c]pyrrolo[3,2-e]pyrimidine;
8-(Pyridin-4-yl)-7H-imidazo[1,2-c]pyrrolo[3,2-e]pyrimidine or 1-(3-(3-(4-isopropylphenyl)-7H-imidazo[1,5-c]pyrrolo[3,2-e]pyrimidin-1-yl)piperidin-1-yl)ethanone.
